(19) United States
(12) Patent Application Publication
(10) Pub. No.: US 2019/0374650 A1
(43) Pub. Date: Dec. 12, 2019

(54) COMPOSITIONS AND METHODS FOR DELIVERY OF POLYMER/BIOMACROMOLECULE CONJUGATES

(71) Applicant: THE REGENTS OF THE UNIVERSITY OF MICHIGAN, Ann Arbor, MI (US)

(72) Inventors: James J. Moon, Ann Arbor, MI (US); Yuchen FAN, Ann Arbor, MI (US); Jutaek NAM, Ann Arbor, MI (US)

(21) Appl. No.: 16/488,031

(22) PCT Filed: Feb. 21, 2018
(86) PCT No.: PCT/US2018/019003

§ 371 Date: Aug. 22, 2019

Related U.S. Application Data
(60) Provisional application No. 62/462,079, filed on Feb. 22, 2017.

(51) Int. Cl. A61K 47/59; A61K 39/00; A61K 39/39; A61K 45/06; A61P 37/04; A61P 35/00

(52) U.S. Cl. CPC A61K 47/593; A61K 39/0011; A61K 39/39; A61K 45/06; A61K 2039/55561; A61P 35/00; A61K 47/59; A61K 2039/6093; A61P 37/04

(57) ABSTRACT

The present invention relates to polymers associated with (e.g., complexed, conjugated, encapsulated, absorbed, adsorbed, admixed) bio macromolecule agents (e.g., peptides, neo-antigens, adjuvant molecules, nucleic acids, etc.) configured for treating, preventing or ameliorating various types of disorders, and methods of synthesizing the same. In particular, the present invention is directed to compositions comprising polymer moieties (e.g., poly(L-glutamic acid moieties)) associated with biomacromolecule agents, methods for synthesizing such polymer conjugates, as well as systems and methods utilizing such polymer conjugates.

### CROSS-REFERENCE TO RELATED APPLICATIONS

This application claims priority to U.S. Provisional Patent Application No. 62/462,079, filed Feb. 22, 2017, which is incorporated herein by reference in its entirety.

### STATEMENT REGARDING FEDERALLY SPONSORED RESEARCH OR DEVELOPMENT

This invention was supported by NIH grant EB022563 and Army/MRMC grant W81XWH-16-1-0369. The government has certain rights in the invention.

### FIELD OF THE INVENTION

The present invention relates to polymers associated with (e.g., complexed, conjugated, encapsulated, absorbed, adsorbed, admixed) biomacromolecule agents (e.g., peptides, neo-antigens, adjuvant molecules, nucleic acids, etc.) configured for treating, preventing or ameliorating various types of disorders, and methods of synthesizing the same. In particular, the present invention is directed to compositions comprising polymer moieties (e.g., poly(L-glutamic acid moieties)) associated with biomacromolecule agents, methods for synthesizing such polymer conjugates, as well as systems and methods utilizing such polymer conjugates.

### BACKGROUND OF THE INVENTION

Subunit vaccines composed of molecularly defined antigens such as peptides, proteins, or nucleotides can relieve reactogenicity and toxicity often induced by live-attenuated whole-cell or whole-virus vaccines, thus offering a promising approach for vaccine development. However, subunit antigens suffer from low immunogenicity in vivo. Hence, subunit vaccines require additional adjuvants as well as delivery strategies to improve vaccine efficacy. Specifically, oligopeptide antigens are subjected to low draining efficiency to lymphoid tissues from injection sites, while peptide sequences of low aqueous solubility further limit lymphoid draining and raise formulation concerns. To address these challenges, subunit antigens have been encapsulated into nanoparticulate delivery systems or conjugated to biopolymers, resulting in enhanced lymphoid draining due to their high molecular weight and water solubility. However, clinical translation of such approaches have been very limited due to (1) manufacturing challenges of scale-up production of nanoparticles and biopolymers in a reproducible manner; (2) difficulty of reliably synthesizing vaccine platforms incorporated with a wide range of oligopeptide antigens, including personalized cancer neo-antigens; and (3) limited in vivo efficacy to generate cellular and humoral immune responses.

Accordingly, there is an unmet medical need for a new generalizable strategy for improving efficacy of oligopeptide antigens.

### SUMMARY

Experiments conducted during the course of developing embodiments for the present invention demonstrated that poly(L-glutamic acid) (PGA) can be readily coupled with antigen (Ag) peptides, including neo-antigens, and adjuvants, producing stable PGA-antigen conjugates that markedly improve Ag/adjuvant co-delivery to lymphoid organs and achieve sustained Ag presentation on dendritic cells. Moreover, it was shown that the PGA platform can be easily adapted to neoantigens, generating potent anti-tumor immunity (see, Example I).

Experiments conducted during the course of developing embodiments for the present invention further demonstrated the therapeutic efficacy of a neo-antigen vaccine (peptide antigens and tumor neo-antigens conjugated on PGA polypeptides) tested on a MC38 tumor model (see, Example II). Experiments conducted during the course of developing embodiments for the present invention further demonstrated cancer immunotherapy with PEI-neo-antigen peptide conjugates (see, Example III).

Accordingly, the present invention relates to polymers associated with (e.g., complexed, conjugated, encapsulated, absorbed, adsorbed, admixed) biomacromolecule agents (e.g., peptides, neo-antigens, adjuvant molecules, nucleic acids, etc.) configured for treating, preventing or ameliorating various types of disorders, and methods of synthesizing the same. In particular, the present invention is directed to compositions comprising polymer moieties (e.g., poly(L-glutamic acid moieties)) associated with biomacromolecule agents, methods for synthesizing such polymer conjugates, as well as systems and methods utilizing such polymer conjugates.

In certain embodiments, the present invention provides compositions comprising a polymer moiety comprising a polymer associated with (e.g., complexed, conjugated, encapsulated, absorbed, adsorbed, admixed) any kind of biomacromolecule agent (e.g., nucleic acid, peptides, glycolipids, etc.).

Such embodiments are not limited to a specific type of polymer comprised within the polymer moiety. Indeed, any type or kind of polymer would find use within various embodiments of the present invention. Examples of such polymer moieties include, but are not limited to, poly(L-glutamic acid (PGA), polyethyleneimine (PEI), poly(gamma-glutamic acid), polyglycolic acid, polyglycolic acid trimethyl-carbonates, polyglycolic acidtrimethyl-carbonates, polyglycolides, copolymers of the polylactides and polyglycolides, polylactic acid (PLLA), poly(lactic-co-glycolic acid) poly(D,L lactic-co-glycolide) (PLGA), PLGA derivatives, polyacrylic acid, polyethylene glycol (PEG), PEG derivatives, methoxy polyethylene glycol (mPEG), poly(D,L-lactide)-block-methoxy polyethylene glycol (diblock), polyethyleneoxide-propyleneoxide, polyethylene oxide (PEO), polyether esters (e.g., polyethyleneoxide, polyalkeneoxalates, polyorthoesters as well as their copolymers), poly-L-lysine, chitosan, hyaluronic acid, fatty acids, fatty acyls, polyamine, polyvinyl alcohol, polyvinylalcoholes, polyvinyl pyrrolidinone, acrylic acid ethylene co-polymer, poly(polypropylacrylic acid) (PPAA), polyacrylic acid, methoxy polyethylene, albumin, human serum albumin, cellulose derivatives, cyclodextrins and their derivatives (e.g., including polytryptophan, polytyrosine, polypropyleneoxide), folate linked cyclodextrin, folate linked cyclodextran, β-cyclodextrines, alginate, carrageenan, pectin, dextran, collagen, collagen-N-hydroxysuccinimide, polyaniline, polyalanine, polytryptophan, polytyrosine, polyamino acids, amino sugars, glycerolipid, glycerophospholipid, sphinglipid, sterol lipid, prenol lipid, saccarolipid, polyketide, glucosamines, lipopolysaccarides, aminopolysaccarides, poly(allylamines), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(ethyleneimine), poly(beta-aminoester), poly(hydroxyethyl methacrylate), polyacrylamide, polyethyloxazole, polysaccharides (e.g., dextran), linear and branched polyethylenimine, polyamidoamine, polypropylenimine, polyallylamine, spermine, polyvalerolactones, poly-ε-decalactones, polylactides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides (e.g., polymaleic anhydrides), polyhydroxymethacrylates, fibrin, polycyanoacrylates, polycaprolactonedimethylacrylates, poly-β-maleic acid, polycaprolactonebutyl-acrylates, multiblock polymers (e.g., oligocaprolactonedioles and oligodioxanonedioles), polyetherester multiblock polymers (e.g., PEG and poly(butyleneterephtalates)), polypivotolactones, polycaprolactone-glycolides, poly(g-ethylglutamate), poly(DTH-iminocarbonate), poly(DTE-co-DT-carbonate), poly(bisphenol-A-iminocarbonate), polyorthoesters, polytrimethylcarbonates, polyiminocarbonates, poly(N-vinyl)-pyrrolidone, polyesteramides, glycolated polyesters, polyphosphoesters, polyphosphazenes, poly[p-carboxyphenoxy]propane], polyhydroxypentanoic acid, soft polyurethanes, polyurethanes having amino acid residues in the backbone, fibrinogen, starch, protein based polymers, polyamino acids, synthetic polyamino acids, zein, modified zein, polyhydroxyalkanoates, pectic acid, actinic acid, modified and non modified fibrin and casein, carboxymethylsulfate, heparansulfates, heparin, chondroitinesulfate, copolymers with PEG and polypropyleneglycol, gummi arabicum, guar, gelatine, lipids and lipoids, polymerizable oils having a low degree of cross-linking, modifications, grafted polymers, and copolymers and/or mixtures of the aforementioned substances.

In some embodiments, the size of the polymer moiety is between approximately 1 KDa to 5000 KDa.

In some embodiments, the biomacromolecule agent is a peptide.

For example, in some embodiments, the peptide is an antigen. In some embodiments wherein the peptide is an antigen, the composition further comprises an adjuvant (as described herein).

In some embodiments, the peptide is Adrenocorticotropic Hormone (ACTH), a growth hormone peptide, a Melanocyte Stimulating Hormone (MSH), Oxytocin, Vasopressin, Corticotropin Releasing Factor (CRF), a CRF-related peptide, a Gonadotropin Releasing Hormone Associated Peptide (GAP), Growth Hormone Releasing Factor (GRF), Lutenizing Hormone Release Hormone (LH-RH), an orexin, a Prolactin Releasing Peptide (PRP), a somatostatin, Thyrotropin Releasing Hormone (THR), a THR analog, Calcitonin (CT), a CT-precursor peptide, a Calcitonin Gene Related Peptide (CGRP), a Parathyroid Hormone (PTH), a Parathyroid Hormone Related Protein (PTHrP), Amylin, Glucagon, Insulin, an Insulin-like peptide, NeuroPeptide Y (NPY), a Pancreatic Polypeptide (PP), Peptide YY (PYY), Cholecystokinin (CCK), a CCK-related peptide, Gastrin Releasing Peptide (GRP), Gastrin, a Gastrin-related peptide, a Gastrin inhibitory peptide, Motilin, Secretin, Vasoactive Intestinal Peptide (VIP), a VIP-related peptide, an Atrial-Natriuretic Peptide (ANP), a Brain Natriuretic Peptide (BNP), a C-Type Natriuretic Peptide (CNP), a tachykinin, an angiotensin, a renin substrate, a renin inhibitor, an endothelin, an endothelin-related peptide, an opioid peptide, a thymic peptide, an adrenomedullin peptide, an allostatin peptide, an amyloid beta-protein fragment, an antimicrobial peptide, an antioxidant peptide, an apoptosis related peptide, a Bag Cell Peptide (BCPs), Bombesin, a bone Gla protein peptide, a Cocaine and Amphetamine Related Transcript (CART) peptide, a cell adhesion peptide, a chemotactic peptide, a complement inhibitor, a cortistatin peptide, a fibronectin fragment, a fibrin related peptide, FMRF, a FMRF amide-related peptide (FaRP), Galanin, a Galanin-related peptide, a growth factor, a growth factor-related peptide, a G-Therapeutic Peptide-Binding Protein fragment, Gualylin, Uroguanylin, an Inhibin peptide, Interleukin (IL), an Interleukin Receptor protein, a laminin fragment, a leptin fragment peptide, a leucokinin, Pituitary Adenylate Cyclase Activating Polypeptide (PAPCAP), Pancreastatin, a polypeptide repetitive chain, a signal transducing reagent, a thrombin inhibitor, a toxin, a trypsin inhibitor, a virus-related peptide, an adjuvant peptide analog, Alpha Mating Factor, Antiarrhythmic Peptide, Anorexigenic Peptide, Alpha-1 Antitrypsin, Bovine Pineal Antireproductive Peptide, Bursin, C3 Peptide P16, Cadherin Peptide, Chromogranin A Fragment, Contraceptive Tetrapeptide, Conantokin G, Conantokin T, Crustacean Cardioactive Peptide, C-Telopeptide, Cytochrome b588 Peptide, Decorsin, Delicious Peptide, Delta-Sleep-Inducing Peptide, Diazempam-Binding Inhibitor Fragment, Nitric Oxide Synthase Blocking Peptide, OVA Peptide, Platelet Calpain Inhibitor (P1), Plasminogen Activator Inhibitor 1, Rigin, Schizophrenia Related Peptide, Sodium Potassium Atherapeutic Peptidase Inhibitor-1, Speract, Sperm Activating Peptide, Systemin, a Thrombin receptor agonist, Tuftsin, Adipokinetic Hormone, Uremic Pentapeptide, Antifreeze Polypeptide, Tumor Necrosis Factor (TNF), Leech [Des Asp10]Decorsin, L-Ornithyltaurine Hydrochloride, P-Aminophenylacetyl Tuftsin, Ac-Glu-Glu-Val-Val-Ala-Cys-pNA, Ac-Ser-Asp-Lys-Pro, Ac-rfwink-NH2, Cys-Gly-Tyr-Gly-Pro-Lys-Lys-Lys-Arg-Lys-Val-Gly-Gly, D-Ala-Leu, D-D-D-D-D, D-D-D-D-D-D, N—P—N-A-N—P-N-A, V-A-I-T-V-L-V-K, V-G-V-R-V-R, V-I-H-S, V-P-D-P-R, Val-Thr-Cys-Gly, R-S-R, Sea Urchin Sperm Activating Peptide, a SHU-9119 antagonist, a MC3-R antagonist, a MC4-R antagonist, Glaspimod, HP-228, Alpha 2-Plasmin Inhibitor, APC Tumor Suppressor, Early Pregnancy Factor, Gamma Interferon, Glandular Kallikrei N-1, Placental Ribonuclease Inhibitor, Sarcolecin Binding Protein, Surfactant Protein D, Wilms' Tumor Suppressor, GABAB 1b Receptor Peptide, Prion Related Peptide (iPRP13), Choline Binding Protein Fragment, Telomerase Inhibitor, Cardiostatin Peptide, Endostatin Derived Peptide, Prion Inhibiting Peptide, N-Methyl D-Aspartate Receptor Antagonist, C-Peptide-Analog, myelin basic protein (MBP), myelin oligodendrocyte protein (MOG), and proteolipid protein (PLP), insulin, and their mimotopes.

In some embodiments, the peptide is selected from 177Lu-DOTA0-Tyr3-Octreotate, Abarelix acetate, ADH-1, Afamelanotidec, melanotan-1, CUV1647, Albiglutide, Aprotinin, Argipressin, Atosiban acetate, Bacitracin, Bentiromide, a BH3 domain, Bivalirudin, Bivalirudin trifluoroacetate hydrate, Blisibimod, Bortezomib, Buserelin, Buserelin acetate, Calcitonin, Carbetocin, Carbetocin acetate, Cecropin A and B, Ceruletide, Ceruletide diethylamine, Cetrorelix, Cetrorelix acetate, Ciclosporine, Cilengitidec, EMD121974, Corticorelin acetate injection, hCRF, Corticorelin ovine triflutate, corticorelin trifluoroacetate, Corticotropin, Cosyntropin, ACTH 1-24, tetracosactide hexaacetate, Dalbavancin, Daptomycin, Degarelix acetate, Depreotide trifluoroacetate (plus sodium pertechnetate), Desmopressin acetate, Desmopressin DDAVP, Dulaglutide, Ecallantide, Edotreotide (plus yttrium-90), Elcatonin acetate, Enalapril maleate (or 2-butanedioate), Enfuvirtide, Eptifibatide, Exenatide, Ganirelix acetate, Glatiramer acetate, Glutathion, Gonadorelin, Gonadorelin acetate, GnRH, LHRH, Goserelin, Goserelin acetate, Gramicidin, Histrelin acetate, Human calcitonin, Icatibant, Icatibant acetate, IM862, oglufanide disodium, KLAKLAK, Lanreotide acetate, Lepirudin, Leuprolide, Leuprolide acetate, leuprorelin, Liraglutide, Lisinopril, Lixisenatide, Lypressin, Magainin2, MALP-2Sc, macrophage-activating lipopeptide-2 synthetic, Nafarelin acetate, Nesiritide, NGR-hTNF, Octreotide acetate, Oritavancin, Oxytocin, Pasireotide, Peginesatide, Pentagastrin, Pentetreotide (plus indium-111), Phenypressin, Pleurocidin, Pramlintide, Protirelin, thyroliberin, TRH, TRF, Salmon calcitonin, Saralasin acetate, Secretin (human), Secretin (porcine), Semaglutide, Seractide acetate, ACTH, corticotropin, Sermorelin acetate, GRF 1-29, Sinapultide, KL4 in lucinactant, Sincalide, Somatorelin acetate, GHRH, GHRF, GRF, Somatostatin acetate, Spaglumat magnesium (or sodium) salt, Substance P, Taltirelin hydrate, Teduglutide, Teicoplanin. Telavancin, Teriparatide, Terlipressin acetate, Tetracosactide, Thymalfasin, thymosin a-1, Thymopentin, Trebananib, Triptorelin, Triptorelin pamoate, Tyroserleutide, Ularitide, Vancomycin, Vapreotide acetate, Vasoactive intestinal peptide acetate, Vx-001c, TERT572Y, Ziconotide acetate, α5-α6 Bax peptide, and β-defensin.

In some embodiments, the peptide is any peptide which would assist in achieving a desired purpose with the composition. For example, in some embodiments, the peptide is any peptide that will facilitate treatment of any type of disease and/or disorder (e.g., peripheral ischemia, cancer, inflammatory disorders, genetic disorders, etc.).

In some embodiments, the biomacromolecule agent is a nucleic acid. Such embodiments encompass any type of nucleic acid molecule including, but not limited to, RNA, siRNA, microRNA, interference RNA, mRNA, replicon mRNA, RNA-analogues, DNA, and DNA analogues.

In certain embodiments, the present invention provides methods for treating conditions, disorders and/or diseases with such compositions comprising a polymer comprising a polymer associated with a biomacromocule agent.

The present invention is not limited to specific types of conditions, disorders and/or diseases.

In some embodiments, the condition, disorder and/or disease is peripheral ischemia, cancer, an inflammatory disorder, a genetic disorder, etc.

In some embodiments, the condition, disorder and/or disease is selected from erythropoietic porphyries, T2 diabetes, antifibrinolytic, central diabetes insipidus, delaying the birth in case of threat of premature birth, antibiotic, cystic fibrosis, angina, anticoagulant in patients with unstable angina undergoing PTCA or PCI, systemic lupus erythematosus, hypercalcemia, osteoporosis, pagets disease, carbetocin works as an oxytocic, antihemorrhagic and uterotonic drug in the peripheral nervous system, prevention of uterine atony, induction, and control postpartum bleeding or haemorrhage, stimulant of the gastric secretion, for treat hormone-sensitive cancers of the prostate and breast, inhibition of premature LH surges in women undergoing controlled ovarian stimulation, immunosuppression in organ transplantation to prevent rejection, peritumoral brain edema, diagnosis of ACTHdependent Cushing's syndrome, allergies, ankylosing spondylitis, psoriasis, chorioditis, erythema, keratitis, sclerosis, dermatomyositis, rheumatoid arthritis, Stevens-Johnson Syndrome, ulcerative colitis, diagnosis of adrenocortical insufficiency, antibiotic, systemic infections caused by gram positive organisms, nocturnal enuresis, nocturia, and stoppage of bleeding or haemorrhage in haemophilia A patients, acute hereditary angioderma, postmenopausal osteoporosis, anti-parathyroid, Paget's disease, hypercalcaemia, hypertension, AIDS/HIV-1 infection, acute coronary syndrome, unstable angina undergoing PCI, Alzheimer's and Parkinson's disease, inhibition of premature LH surges in women undergoing controlled ovarian hyperstimulation, Relapsing-Remitting Multiple Sclerosis, hepatic insufficiency, wound healing, inflammation of respiratory tract, asthenia, release of follicle-stimulating hormone (FSH) and luteinizing hormone (LH) from the anterior pituitary, stimulate the secretion of gonadotropin during disturbances fertility, and diagnosis of the functional capacity and response of the gonadotropes of the anterior pituitary, for skin lesions, surface wounds and eye infections, postmenopausal osteoporosis, Paget's disease, hypercalcaemia, hereditary angioedema, immune system related diseases, acromegaly, anticoagulant, fibroids and endometriosis, central diabetes insipidus, Cushing's syndrome, diabetic foot ulcers, treatment of central precocious puberty, uterine fibriods and endometriosis, vasodilatory, natriuretic, diuretic and neurohormonal effects, acromegaly, carcinoid syndrome, acute bacterial skin and skin structure infections, initiation or improvement of uterine contractions, and control postpartum bleeding or haemorrhage, hematide Chronic kidney disease associated anemia, stomatitis, pharyngitis, diagnostic assessment of thyroid function, postmenopausal osteoporosis, hypercalcaemia, diagnosis of pancreatic exocrine dysfunction, and gastrinoma, Zollinger-Ellison syndrome, prevention of RDS in premature infants, and meconium aspiration syndrome, acute variceal bleeding, allergic rhinitis and conjunctivitis, spinocerebellar degeneration/ataxia, Short Bowel Syndrome, antibiotic, bactericidal, teriparatide is the only anabolic (i.e., bone growing) agent indicated for use in postmenopausal women with osteoporosis, Cortrosyn Analogue of adrenocorticotrophic hormone (ACTH) used for diagnostic purposes, treatment of adrenal insufficiency, epilepsia, Chronic hepatitis B, chronic hepatitis C, primary and secondary immune deficiencies, acute decompensated heart failure, colitis, esophageal variceal bleeding in patients with cirrhotic liver disease and AIDS-related diarrhea, sarcoidosis and acute lung injury, and severe chronic pain.

In some embodiments, the peptide and condition, disorder and/or disease to be treated is shown in each row of Table 1. Table 1 provides a list of therapeutic peptides and conditions, disorders, and/or diseases targeted by such therapeutic peptides (e.g., each row presents a therapeutic peptide and the condition, disorder and/or disease targeted by the respective therapeutic peptide).

TABLE 1

| Therapeutic Peptide | Target Condition, Disorder and/or Disease |
|---|---|
| 177Lu-DOTA0-Tyr3- | Midgut carcinoid tumors |
| Octreotate | |
| Abarelix acetate | Advanced prostate cancer |
| ADH-1 | Malignant melanoma (FDA orphan drug status |
| | Phase II) |
| Afamelanotidec, or | Erythropoietic porphyries (EMEA and FDA |
| melanotan-1, or CUV1647 | orphan drug status Phase III) |
| Albiglutide | T2 diabetes |
| Aprotinin | Antifibrinolytic |
| Argipressin | Central diabetes insipidus, and BOV |
| Atosiban acetate | Delaying the birth in case of threat of premature birth |
| Bacitracin | Antibiotic |
| Bentiromide | Approved by FDA for screening test of exocrine |
| | pancreatic insufficiency in patients with cystic fibrosis |
| BH3 domain | Cancer: apoptosis |
| Bivalirudin | Direct thrombin inhibitor |
| Bivalirudin | Angina |
| Bivalirudin trifluoroacetate | Anticoagulant in patients with unstable angina |
| hydrate | undergoing PTCA or PCI |
| Blisibimod | Systemic lupus erythematosus |
| Bortezomib | Multiple myeloma, and refractory, mantle cell |
| | lymphoma |
| Buserelin | Treatment of prostate and breast cancer |
| Buserelin acetate | Advanced prostate cancer |
| Calcitonin | Hypercalcemia, osteoporosis, pagets disease |
| Carbetocin | Carbetocin works as an oxytocic, antihemorrhagic |
| | and uterotonic drug in the peripheral nervous |
| | system |
| Carbetocin acetate | Prevention of uterine atony, induction, and control |
| | postpartum bleeding or haemorrhage |
| Cecropin A and B | Leukemia; Bladder cancer |
| Ceruletide | Potent cholecystokinetic agent with a direct |
| | spasmogenic effect on the gallbladder muscle |
| | and bile ducts in humans and animals |
| Ceruletide diethylamine | Diagnosis of the functional state of the |
| | gallbladder and pancreas, and stimulant of the |
| | gastric secretion |
| Cetrorelix | GnRH antagonist, used to treat hormone- |
| | sensitive cancers of the prostate and breast |
| Cetrorelix acetate | Inhibition of premature LH surges in women |
| | undergoing controlled ovarian stimulation |
| Ciclosporine | An immunosuppressant drug widely used in |
| | organ transplantation to prevent rejection |
| Cilengitidec, or | GBM (EMEA and FDA orphan drug status - |
| EMD121974 | Phase III) |
| Corticorelin acetate | Peritumoral brain edema (FDA orphan drug |
| injectionc, or hCRF | status - Phase III) |
| Corticorelin ovine triflutate, | Diagnosis of ACTHdependent Cushing's |
| or corticorelin | syndrome |
| trifluoroacetate | |
| Corticotropin | Allergies, ankylosing spondylitis, psoriasis, |
| | chorioditis, erythema, keratitis, sclerosis, |
| | dermatomyositis, rheumatoid arthritis, Stevens- |
| | Johnson Syndrome, Systemic Lupus |
| | Erythematosus, Ulcerative Colitis |
| Cosyntropin, or ACTH 1- | Diagnosis of adrenocortical insufficiency |
| 24, or tetracosactide | |
| hexaacetate | |
| Dalbavancin | antibiotic |
| Daptomycin | Systemic infections caused by gram positive |
| | organisms |
| Degarelix acetate | Treatment of prostate cancer |
| Depreotide trifluoroacetate | Diagnosis (scintigraphic imaging) of lung tumours |
| (plus sodium | |
| pertechnetate) | |
| Desmopressin acetate | insipidus, nocturnal enuresis, nocturia, and |
| | stoppage of bleeding or haemorrhage in |
| | haemophilia A patients |
| Desmopressin DDAVP | To treat nocturnal enuresis (bedwetting) |
| Dulaglutide | Type 2 diabetes mellitus |
| Ecallantide | Acute hereditary angioderma |
| Edotreotide (plus yttrium-90) | Gastro-entero-pancreatic neuroendocrine |
| | tumours (FDA orphan drug status - Phase II) |
| Elcatonin acetate | Postmenopausal osteoporosis, anti-parathyroid, |
| | Paget's disease, hypercalcaemia |
| Enalapril maleate (or 2- | Hypertension |
| butanedioate) | |
| Enfuvirtide | AIDS/HIV-1 infection |
| Eptifibatide | Acute coronary syndrome, unstable angina |
| | undergoing PCI |
| Exenatide | Type 2 diabetes. Preclinical Studies also revealed |
| | its neuroprotective role in Alzheimer's and |
| | Parkinson's disease |
| Ganirelix acetate | Inhibition of premature LH surges in women |
| | undergoing controlled ovarian hyperstimulation |
| Glatiramer acetate | Reduction of the frequency of relapses in patients |
| | with Relapsing - Remitting Multiple Sclerosis |
| Glutathion | Hepatic insufficiency, wound healing, |
| | inflammation of respiratory tract, asthenia |
| Gonadorelin | Release of follicle-stimulating hormone (FSH) and |
| | luteinizing hormone (LH) from the anterior |
| | pituitary |
| Gonadorelin acetate, or | Stimulate the secretion of gonadotropin during |
| GnRH, or LHRH | disturbances fertility, and diagnosis of the |
| | functional capacity and response of the |
| | gonadotropes of the anterior pituitary |
| Goserelin | Breast cancer and prostate cancer |
| Goserelin acetate | Advanced prostate cancer, breast cancer |
| Gramicidin | Used to treat skin lesions, surface wounds and |
| | eye infections |
| Histrelin acetate | Treatment of prostate and breast cancer |
| Human calcitonin | Postmenopausal osteoporosis, Paget's disease, |
| | hypercalcaemia |
| Icatibant | Hereditary angioedema |
| Icatibant acetate | Hereditary angioedema |
| IM862, or oglufanide | Immune system related diseases (FDA orphan |
| disodium | drug status for ovarian cancer - Phase II) |
| KLAKLAK | glioblastoma |
| Lanreotide acetate | Acromegaly |
| Lepirudin | Anticoagulant |
| Leuprolide | Treatment of prostate and breast cancer, and to |
| | treat fibroids and endometriosis, Alzheimer |
| | disease |
| Leuprolide acetate, or | Advanced prostate cancer, breast cancer, central |
| leuprorelin | precocious puberty |
| Liraglutide | Type 2 diabetes |
| Lisinopril | Hypertension, congestive heart failure |
| Lixisenatide | Treatment of Diabetes |
| Lypressin | Central diabetes insipidus, Cushing's syndrome |
| Magainin2 | Bladder cancer; Diabetic foot ulcers |
| MALP-2Sc, or | Pancreatic cancer (EMEA orphan drug status - |
| macrophage-activating | Phase II) |
| lipopeptide-2 synthetic | |
| Nafarelin acetate | Treatment of central precocious puberty, uterine |
| | fibriods and endometriosis |
| Nesiritide | Vasodilatory, natriuretic, diuretic and |
| | neurohormonal effects |
| NGR-hTNF | Mesothelioma |
| Octreotide acetate | Acromegaly, carcinoid syndrome |
| Oritavancin | Acute bacterial skin and skin structure infections |
| Oxytocin | Initiation or improvement of uterine contractions, |
| | and control postpartum bleeding or haemorrhage |
| Pasireotide | Cushing's disease and Acromegaly |
| Peginesatide | Hematide Chronic kidney disease associated |
| | anemia |
| Pentagastrin | Diagnosis of the gastric secretion |
| Pentetreotide (plus indium- | Diagnosis (scintigraphic imaging) of primary and |
| 111) | metastatic neuroendocrine tumours |
| Phenypressin | Stomatitis, pharyngitis |
| Pleurocidin | Breast cancer |
| Pramlintide | Diabetes |
| Protirelin, or thyroliberin, or | Diagnostic assessment of thyroid function |
| TRH, or TRF | |
| Salmon calcitonin | Postmenopausal osteoporosis, Paget's disease, |
| | hypercalcaemia |
| Saralasin acetate | Hypertension |
| Secretin (human) | Diagnosis of pancreatic exocrine dysfunction, and |
| | gastrinoma, Zollinger-Ellison syndrome |
| Secretin (porcine) | Diagnosis of pancreatic exocrine dysfunction, and |
| | gastrinoma, Zollinger-Ellison syndrome |
| Semaglutide | T2 diabetes |
| Seractide acetate, or | Diagnosis of adrenocortical insufficiency |
| ACTH, or corticotropin | |
| Sermorelin acetate or GRF | Growth hormone deficiency, diagnosis evaluation |
| 1-29 | of pituitary function |
| Sinapultide, or KL4 in | Prevention of RDS in premature infants, and |
| lucinactant | meconium aspiration syndrome |
| Sincalide | Diagnosis of the functional state of the |
| | gallbladder and pancreas, and stimulant of the |
| | gastric secretion |
| Somatorelin acetate, or | Diagnosis of somatotropic function of the anterior |
| GHRH, or GHRF, or GRF | pituitary gland in cases of suspected growth |
| | hormone deficiency (hypophysic and |
| | hypothalamic disorders) |
| Somatostatin acetate | Acute variceal bleeding |
| Spaglumat magnesium (or | Allergic rhinitis and conjunctivitis |
| sodium) salt | |
| Taltirelin hydrate | Spinocerebellar degeneration/ataxia |
| Teduglutide | Short Bowel Syndrome (EMEA and FDA orphan |
| | drug status - Phase III) |
| Teicoplanin | Antibiotic |
| Telavancin | bactericidal |
| Teriparatide | Teriparatide is the only anabolic (i.e., bone |
| | growing) agent indicated for use in |
| | postmenopausal women with osteoporosis |
| Terlipressin acetate | BOV |
| Tetracosactide | Cortrosyn Analogue of adrenocorticotrophic |
| | hormone (ACTH) used for diagnostic purposes, |
| | treatment of adrenal insufficiency, different types |
| | of drug registant epilepsia |
| Thymalfasin, or thymosin a-1 | Chronic hepatitis B, chronic hepatitis C |
| Thymopentin | Primary and secondary immune deficiencies, |
| | autoimmunity, infections, cancer |
| Trebananib | Ovarian, peritoneal or fallopian tube cancers |
| Triptorelin | Trelstar Treatment of prostate and breast cancer |
| Triptorelin pamoate | Advanced prostate cancer, central precocious |
| | puberty, endometriosis, uterine fibroids, ovarian |
| | stimulation in in vitro fecundation |
| Tyroserleutide | Hepatocellular carcinoma |
| Ularitide | Acute decompensated heart failure |
| Vancomycin | Treat colitis (inflammation of the intestine caused |
| | by certain bacteria) that may occur after antibiotic |
| | treatment |
| Vapreotide acetate | Treatment of esophageal variceal bleeding in |
| | patients with cirrhotic liver disease and AIDS- |
| | related diarrhea |
| Vasoactive intestinal | Sarcoidosis and acute lung injury (EMEA and |
| peptide acetate | FDA orphan drug status Phase II) |
| Vx-001c or TERT572Y | NSCLC (EMEA and FDA orphan drug status - |
| | Phase II) |
| Ziconotide acetate | Severe chronic pain |
| α5-α6 Bax peptide | Cancer: apoptosis |
| β-defensin | Antimicrobial |

In some embodiments, composition is co-administered with a chemotherapeutic agent. In some embodiments, the chemotherapeutic agent is one or more of the following: aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate.

In certain embodiments, the present invention provides methods for making a personalized neoplasia vaccine for a subject diagnosed as having a neoplasia. The present invention is not limited to particular methods for making a personalized neoplasia vaccine for a subject diagnosed as having a neoplasia. In some embodiments, such methods comprise obtaining a biological sample of the neoplasia from the subject; identifying a plurality of mutations in the neoplasia; analyzing the plurality of mutations to identify one or more neo-antigenic mutations predicted to encode neo-antigenic peptides, the neo-antigenic mutations selected from the group consisting of missense mutations, neoORF mutations, and any combination thereof; and producing a personalized neoplasia vaccine, wherein the personalized neoplasia vaccine comprises a polymer moiety comprising a polymer associated with (e.g., complexed, conjugated, encapsulated, absorbed, adsorbed, admixed) one or more neo-antigenic peptides specific for the analyzed and identified neo-antigenic mutations predicted to encode neo-antigenic peptides. In some embodiments, the polymer is further associated with (e.g., complexed, conjugated, encapsulated, absorbed, adsorbed, admixed) with an adjuvant. In some embodiments, the identifying further comprises sequencing the genome, transcriptome, or proteome of the neoplasia. In some embodiments, the subject is a human being.

In certain embodiments, the present invention provides a composition comprising a polymer moiety obtained through use of such methods.

In some embodiments, the neo-antigenic peptide is conjugated to the outer surface of the polymer. In some embodiments, the adjuvant is conjugated to the outer surface of the polymer. In some embodiments, the adjuvant is encapsulated within or complexed with the polymer. Such embodiments are not limited to a specific manner of conjugation. In some embodiments, the conjugation is via a reduction sensitive linkage (e.g., pyridyldithiol propionate-thiol linkage). In some embodiments, the conjugation is via a reduction insensitive linkage (e.g., maleimide-thiol linkage).

In some embodiments, the size of the polymer moiety is between approximately 1 KDa to 5000 KDa.

Such embodiments are not limited to a specific type of polymer comprised within the polymer moiety. Indeed, any type or kind of polymer would find use within various embodiments of the present invention. Examples of such polymer moieties include, but are not limited to, poly(L-glutamic acid (PGA), polyethyleneimine (PEI), poly(gamma-glutamic acid), polyglycolic acid, polyglycolic acid trimethyl-carbonates, polyglycolic acidtrimethyl-carbonates, polyglycolides, copolymers of the polylactides and polyglycolides, polylactic acid (PLLA), poly(lactic-co-glycolic acid) poly(D,L lactic-co-glycolide) (PLGA), PLGA derivatives, polyacrylic acid, polyethylene glycol (PEG), PEG derivatives, methoxy polyethylene glycol (mPEG), poly(D,L-lactide)-block-methoxypolyethylene glycol (diblock), polyethyleneoxide-propyleneoxide, polyethylene oxide (PEO), polyether esters (e.g., polyethyleneoxide, polyalkeneoxalates, polyorthoesters as well as their copolymers), poly-L-lysine, chitosan, hyaluronic acid, fatty acids, fatty acyls, polyamine, polyvinyl alcohol, polyvinylalcoholes, polyvinyl pyrrolidinone, acrylic acid ethylene co-polymer, poly(polypropylacrylic acid) (PPAA), polyacrylic acid, methoxy polyethylene, albumin, human serum albumin, cellulose derivatives, cyclodextrins and their derivatives (e.g., including polytryptophan, poly tyrosine, polypropyleneoxide), folate linked cyclodextrin, folate linked cyclodextran, β-cyclodextrines, alginate, carrageenan, pectin, dextran, collagen, collagen-N-hydroxysuccinimide, polyaniline, polyalanine, polytryptophan, polytyrosine, polyamino acids, amino sugars, glycerolipid, glycerophospholipid, sphinglipid, sterol lipid, prenol lipid, saccarolipid, polyketide, glucosamines, lipopolysaccarides, aminopolysaccarides, poly(allylamines), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly (hydroxyalkanoates), poly(2-dimethylamino)ethyl methacrylate (DMAEMA), poly(ethyleneimine), poly(beta-aminoester), poly(hydroxyethyl methacrylate), polyacrylamide, polyethyloxazole, polysaccharides (e.g., dextran), linear and branched polyethylenimine, polyamidoamine, polypropylenimine, polyallylamine, spermine, polyvalerolactones, poly-ε-decalactones, polylactides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides (e.g., polymaleic anhydrides), polyhydroxymethacrylates, fibrin, polycyanoacrylates, polycaprolactonedimethylacrylates, poly-β-maleic acid, polycaprolactonebutyl-acrylates, multiblock polymers (e.g., oligocaprolactonedioles and oligodioxanonedioles), polyetherester multiblock polymers (e.g., PEG and poly(butyleneterephtalates)), polypivotolactones, polycaprolactone-glycolides, poly(g-ethylglutamate), poly(DTH-iminocarbonate), poly(DTE-co-DT-carbonate), poly(bisphenol-A-iminocarbonate), polyorthoesters, polytrimethylcarbonates, polyiminocarbonates, poly(N-vinyl)-pyrrolidone, polyesteramides, glycolated polyesters, polyphosphoesters, polyphosphazenes, poly[p-carboxyphenoxy]propane], polyhydroxypentanoic acid, soft polyurethanes, polyurethanes having amino acid residues in the backbone, fibrinogen, starch, protein based polymers, polyamino acids, synthetic polyamino acids, zein, modified zein, polyhydroxyalkanoates, pectic acid, actinic acid, modified and non modified fibrin and casein, carboxymethylsulfate, heparansulfates, heparin, chondroitinesulfate, copolymers with PEG and polypropyleneglycol, gummi arabicum, guar, gelatine, lipids and lipoids, polymerizable oils having a low degree of cross-linking, modifications, grafted polymers, and copolymers and/or mixtures of the aforementioned substances.

In some embodiments, the one or more neo-antigenic peptides range from about 5 to about 50 amino acids in length. In some embodiments, the one or more neo-antigenic mutations peptides range from about 15 to about 35 amino acids in length. In some embodiments, the one or more neo-antigenic peptides range from about 18 to about 30 amino acids in length. In some embodiments, the one or more neo-antigenic peptides range from about 6 to about 15 amino acids in length.

In some embodiments, the adjuvant is selected from the group consisting of CPG, polyIC, poly-ICLC, 1018 ISS, aluminum salts (for example, aluminum hydroxide, aluminum phosphate), Amplivax, BCG, CP-870,893, CpG7909, CyaA, dSLIM, Cytokines (such as GM-CSF, IL-2, IFN-a, Flt-3L), IC30, IC31, Imiquimod, ImuFact IMP321, IS Patch, ISS, ISCOMATRIX, Juylmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), 3M MED19197, glucopyranosyl lipid adjuvant (GLA), GLA-SE, CD1d ligands (such as C20:2, OCH, AH04-2, α-galatosylceramide, α-C-galatosylceramide,α-mannosylceramide, α-fructosylceramide, β-galatosylceramide, β-mannosylceramide), STING agonists (e.g. cyclic dinucleotides, including Cyclic [G(3′,5′)pA(3′,5′)p], Cyclic [G(2′,5′)pA(3′,5′)p], Cyclic [G(2′,5′)pA(2′,5′)p], Cyclic diadenylate monophosphate, Cyclic diguanylate monophosphate), CL401, CL413, CL429, Flagellin, RC529, E6020, imidazoquinoline-based small molecule TLR-7/8a (including its lipidated analogues), virosomes, AS01, AS02. AS03, AS04, AS15, IC31, CAF01, ISCOM, Cytokines (such as GM-CSF, IL-2, IFN-a, Flt-3L), bacterial toxins (such as CT, and LT). In some embodiments, the adjuvant is any derivative of an adjuvant (e.g., cholesterol-modified CpG) or any combinations thereof.

In certain embodiments, the present invention provides methods for treating a subject diagnosed as having a neoplasia with such a personalized neoplasia vaccine. The present invention is not limited to particular methods for treating a subject diagnosed as having a neoplasia with a personalized neoplasia vaccine.

In some embodiments, the personalized neoplasia vaccine is coadministered with an an anti-immunosuppressive or immuno stimulatory agent. In some embodiments, the anti-immunosuppressive or immuno stimulatory agent is selected from the group consisting of anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, and inhibitors of IDO.

In some embodiments, the personalized neoplasia vaccine is co-administered with a chemotherapeutic agent. In some embodiments, the chemotherapeutic agent is one or more of the following: aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate.

In certain embodiments, the present invention provides compositions comprising a polymer moiety comprising a polymer associated with (e.g., complexed, conjugated, encapsulated, absorbed, adsorbed, admixed) an antigen. In some embodiments, the polymer is further associated with an adjuvant. In certain embodiments, the present invention provides methods for inducing an immune response to an antigen comprising administering to a subject in need an effective amount of a composition comprising a polymer moiety comprising a polymer associated with (e.g., complexed, conjugated, encapsulated, absorbed, adsorbed, admixed) an antigen. In some embodiments, the polymer is further associated with an adjuvant.

Such embodiments are not limited to a specific type of polymer comprised within the polymer moiety. Indeed, any type or kind of polymer would find use within various embodiments of the present invention. Examples of such polymer moieties include, but are not limited to, poly(L-glutamic acid (PGA), polyethyleneimine (PEI), poly(gamma-glutamic acid), polyglycolic acid, polyglycolic acid trimethyl-carbonates, polyglycolic acidtrimethyl-carbonates, polyglycolides, copolymers of the polylactides and polyglycolides, polylactic acid (PLLA), poly(lactic-co-glycolic acid) poly(D,L lactic-co-glycolide) (PLGA), PLGA derivatives, polyacrylic acid, polyethylene glycol (PEG), PEG derivatives, methoxy polyethylene glycol (mPEG), poly(D,L-lactide)-block-methoxypolyethylene glycol (diblock), polyethyleneoxide-propyleneoxide, polyethylene oxide (PEO), polyether esters (e.g., polyethyleneoxide, polyalkeneoxalates, polyorthoesters as well as their copolymers), poly-L-lysine, chitosan, hyaluronic acid, fatty acids, fatty acyls, polyamine, polyvinyl alcohol, polyvinylalcoholes, polyvinyl pyrrolidinone, acrylic acid ethylene co-polymer, poly(polypropylacrylic acid) (PPAA), polyacrylic acid, methoxy polyethylene, albumin, human serum albumin, cellulose derivatives, cyclodextrins and their derivatives (e.g., including polytryptophan, polytyrosine, polypropyleneoxide), folate linked cyclodextrin, folate linked cyclodextran, β-cyclodextrines, alginate, carrageenan, pectin, dextran, collagen, collagen-N-hydroxysuccinimide, polyaniline, polyalanine, polytryptophan, polytyrosine, polyamino acids, amino sugars, glycerolipid, glycerophospholipid, sphinglipid, sterol lipid, prenol lipid, saccarolipid, polyketide, glucosamines, lipopolysaccarides, aminopolysaccarides, poly(allylamines), polypropylene glycol (PPG), poly(lactic acid), poly(glycolic acid), poly(ethylene-co-vinyl acetate) (EVAc), N-(2-hydroxypropyl)methacrylamides (HPMA). HPMA derivatives, poly(hydroxyalkanoates), poly(2-di methylamino)ethyl methacrylate (DMAEMA), poly(ethyleneimine), poly(beta-aminoester), poly(hydroxyethyl methacrylate), polyacrylamide, polyethyloazole, polysaccharides (e.g., dextran), linear and branched polyethylenimine, polyamidoamine, polypropylenimine, polyallylamine, spermine, polyvalerolactones, poly-ε-decalactones, polylactides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides (e.g., polymaleic anhydrides), polyhydroxymethacrylates, fibrin, polycyanoacrylates, polycaprolactonedimethylacrylates, poly-β-maleic acid, polycaprolactonebutyl-acrylates, multiblock polymers (e.g., oligocaprolactonedioles and oligodioxanonedioles), polyetherester multiblock polymers (e.g., PEG and poly(butyleneterephtalates)), polypivotolactones, polycaprolactone-glycolides, poly(g-ethylglutamate), poly(DTH-iminocarbonate), poly(DTE-co-DT-carbonate), poly(bisphenol-A-iminocarbonate), polyorthoesters, polytrimethylcarbonates, polyiminocarbonates, poly(N-vinyl)-pyrrolidone, polyesteramides, glycolated polyesters, polyphosphoesters, polyphosphazenes, poly[p-carboxyphenoxy]propane], polyhydroxypentanoic acid, soft polyurethanes, polyurethanes having amino acid residues in the backbone, fibrinogen, starch, protein based polymers, polyamino acids, synthetic polyamino acids, zein, modified zein, polyhydroxyalkanoates, pectic acid, actinic acid, modified and non modified fibrin and casein, carboxymethylsulfate, heparansulfates, heparin, chondroitinesulfate, copolymers with PEG and polypropyleneglycol, gummi arabicum, guar, gelatine, lipids and lipoids, polymerizable oils having a low degree of cross-linking, modifications, grafted polymers, and copolymers and/or mixtures of the aforementioned substances.

In some embodiments, the antigen is selected from the group consisting of a peptide based antigen, a protein based antigen, a polysaccharide based antigen, a saccharide based antigen, a lipid based antigen, a glycolipid based antigen, a nucleic acid based antigen, an inactivated organism based antigen, an attenuated organism based antigen, a viral antigen, a bacterial antigen, a parasite antigen, an antigen derived from an allergen, and a tumor antigen.

In some embodiments, the antigen is against PCSK9, M30, M27, Adpgk, and/or ASMTNMELM.

In some embodiments, the antigen is a tumor antigen selected from the group consisting of alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGS), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), human EGFR protein or its fragments, such as human EGFR residues 306-325 (SCVRACGADSYEMEEDGVRK) and residues 897-915 (VWSYGVTVWELMTFGSKPY), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, TPS, WT1 (and WT1-derived peptide sequences: WT1 126-134 (RMFP NAPYL), WT1 122-140 (SGQARMFPNAPYLPSCLES), and WT1 122-144 (SGQARMFPNAPYLPSCLESQPTI)), MUC1 (and MUC1-derived peptides and glycopeptides such as RPAPGS, PPAHGVT, and PDTRP), LMP2, EGFRvIII, Idiotype, GD2, Ras mutant, p53 mutant, Proteinase3 (PR1), Survivin, hTERT, Sarcoma translocation breakpoints, EphA2, EphA4, LMW-PTP, PAP, ML-IAP, AFP, ERG (TMPRSS2 ETS fusion gene), NA17, PAX3, ALK, Androgen receptor, Cyclin B1, Polysialic acid, MYCN, RhoC, TRP-2, GD3, Fucosyl GMI, Mesothelin, sLe(animal), CYP1B1, PLAC1, GM3, BORIS, Tn, GloboH, NY-BR-1, RGS5, SART3, STn, Carbonic anhydrase IX, PAX5, OY-TES1, Sperm protein 17, LCK, HMWMAA, AKAP-4, XAGE 1, B7H3, Legumain, Tie 2, Page4, VEGFR2, MAD-CT-1, FAP, PDGFR-alpha, PDGFR-β, MAD-CT-2, Fos-related antigen 1, ERBB2, Folate receptor 1 (FOLR1 or FBP), IDH1, IDO, LY6K, fins-related tyro-sine kinase 1 (FLT1, best known as VEGFR1), KDR, PADRE, TA-CIN (recombinant HPV16 L2E7E6), SOX2, aldehyde dehydrogenase, and any derivative thereof.

In some embodiments, the antigen is any type of viral, bacterial or self-antigen including, but not limited to, FimH against urinary tract infection; soluble F protein from respiratory syncytial virus (RSV); NEF, GAG, and ENV protein from HIV; Streptococcus pneumoniae proteins; HMGB1 protein; hemagglutinin and neuroamidase protein against influenza; Viral antigens derived from HPV type 16 and 18; gL2, ICP4, gD2ΔTMR, gD2ΔTMR, or ICP4.2 from HSV-2; antigens from S. pneumoniae, such as a pneumolysoid, Choline-binding protein A (CbpA), or Pneumococcal surface protein A (PspA), SP1912, SP1912, SP1912L, SP0148 with or without a signal sequence, SP2108 with or without a signal sequence; Antigens from Chlamydia trachomatis, such as a CT209 polypeptide antigen, a CT253 polypeptide antigen, a CT425 polypeptide antigen, a CT497 polypeptide antigen, and a CT843 polypeptide antigen; amyloid-beta peptide.

In some embodiments, the adjuvant is a dendritic cell targeting molecule. In some embodiments, the adjuvant is selected from the group consisting of CPG, polyIC, poly-ICLC, 1018 ISS, aluminum salts (for example, aluminum hydroxide, aluminum phosphate), Amplivax, BCG, CP-870,893, CpG7909, CyaA, dSLIM, Cytokines (such as GM-CSF, IL-2, IFN-a, Flt-3L), IC30, IC31, Imiquimod, ImuFact IMP321, IS Patch, ISS, ISCOMATRIX, Juylmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), 3M MEDI9197, glucopyranosyl lipid adjuvant (GLA), GLA-SE, CD1d ligands (such as C20:2. OCH, AH04-2, α-galatosylceramide, α-C-galatosylceramide, α-mannosylceramide, α-fructosylceramide, β-galatosylceramide, β-mannosylceramide), STING agonists (e.g. cyclic dinucleotides, including Cyclic [G(3′,5′)pA(3′,5′)p], Cyclic [G(2′,5′)pA(3′,5′)p], Cyclic [G(2′,5′)pA(2′,5′)p], Cyclic diadenylate monophosphate, Cyclic diguanylate monophosphate), CL401, CL413, CL429, Flagellin, RC529, E6020, imidazoquinoline-based small molecule TLR-7/8a (including its lipidated analogues), virosomes, AS01, AS02, AS03, AS04, AS15, IC31, CAF01, ISCOM, Cytokines (such as GM-CSF, IL-2, IFN-a, Flt-3L), and bacterial toxins (such as CT, and LT). In some embodiments, the adjuvant is any derivative of an adjuvant (e.g., cholesterol-modified CpG) or any combinations thereof.

In some embodiments, the antigen is conjugated to the outer surface of the polymer. In some embodiments, the adjuvant is conjugated to the outer surface of the polymer. In some embodiments, the adjuvant is encapsulated within or complexed with the polymer. Such embodiments are not limited to a specific manner of conjugation. In some embodiments, the conjugation is via a reduction sensitive linkage (e.g., pyridyldithiol propionate-thiol linkage). In some embodiments, the conjugation is via a reduction insensitive linkage (e.g., maleimide-thiol linkage).

In some embodiments, the size of the polymer moiety is between approximately 1 KDa to 5000 KDa.

In some embodiments, the composition comprising a polymer moiety comprising a polymer associated with an antigen is coadministered with an an anti-immunosuppressive or immuno stimulatory agent. In some embodiments, the anti-immunosuppressive or immuno stimulatory agent is selected from the group consisting of anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, and inhibitors of IDO.

In some embodiments, the composition comprising a polymer moiety comprising a polymer associated with an antigen is co-administered with a chemotherapeutic agent. In some embodiments, the chemotherapeutic agent is one or more of the following: aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate.

In certain embodiments, the present invention provides polymer moeities comprising a polymer associated with (e.g., complexed, conjugated, encapsulated, absorbed, adsorbed, admixed) an siRNA.

Such embodiments are not limited to a specific type of polymer comprised within the polymer moiety. Indeed, any type or kind of polymer would find use within various embodiments of the present invention. Examples of such polymer moieties include, but are not limited to, poly(L-glutamic acid (PGA), polyethyleneimine (PEI), poly(gamma-glutamic acid), polyglycolic acid, polyglycolic acid trimethyl-carbonates, polyglycolic acidtrimethyl-carbonates, polyglycolides, copolymers of the polylactides and polyglycolides, polylactic acid (PLLA), poly(lactic-co-glycolic acid) poly(D,L lactic-co-glycolide) (PLGA), PLGA derivatives, polyacrylic acid, polyethylene glycol (PEG), PEG derivatives, methoxy polyethylene glycol (mPEG), poly(D,L-lactide)-block-methoxypolyethylene glycol (diblock), polyethyleneoxide-propyleneoxide, polyethylene oxide (PEO), polyether esters (e.g., polyethyleneoxide, polyalkeneoxalates, polyorthoesters as well as their copolymers), poly-L-lysine, chitosan, hyaluronic acid, fatty acids, fatty acyls, polyamine, polyvinyl alcohol, polyvinylalcoholes, poly vinyl pyrrolidinone, acrylic acid ethylene co-polymer, poly(polypropylacrylic acid) (PPAA), polyacrylic acid, methoxy polyethylene, albumin, human serum albumin, cellulose derivatives, cyclodextrins and their derivatives (e.g., including polytryptophan, poly tyrosine, polypropyleneoxide), folate linked cyclodextrin, folate linked cyclodextran, -cyclodextrines, alginate, carrageenan, pectin, dextran, collagen, collagen-N-hydroxysuccinimide, polyaniline, polyalanine, polytryptophan, polytyrosine, polyamino acids, amino sugars, glycerolipid, glycerophospholipid, sphinglipid, sterol lipid, prenol lipid, saccarolipid, polyketide, glucosamines, lipopolysaccarides, aminopolysaccarides, poly(allylamines), polypropylene glycol (PPG), poly(lactic acid), poly(glycolic acid), poly(ethylene-co-vinyl acetate) (EVAc), N-(2-hydroxy propyl)methacrylamides (HPMA), HPMA derivatives, poly(hydroxy alkanoates), poly(2-dimethylamino)ethyl methacrylate (DMAEMA), poly(ethyleneimine), poly(beta-aminoester), poly(hydroxyethyl methacrylate), polyacrylamide, polyethyloxazole, polysaccharides (e.g., dextran), linear and branched polyethylenimine, polyamidoamine, polypropylenimine, polyallylamine, spermine, polyvalerolactones, poly-ε-decalactones, polylactides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides (e.g., polymaleic anhydrides), polyhydroxymethacrylates, fibrin, polycyanoacrylates, polycaprolactonedimethylacrylates, poly-β-maleic acid, polycaprolactonebutyl-acrylates, multiblock polymers (e.g., oligocaprolactonedioles and oligodioxanonedioles), polyetherester multiblock polymers (e.g., PEG and poly(butyleneterephtalates)), polypivotolactones, polycaprolactone-glycolides, poly(g-ethylglutamate), poly(DTH-iminocarbonate), poly(DTE-co-DT-carbonate), poly(bisphenol-A-iminocarbonate), polyorthoesters, polytrimethylcarbonates, polyiminocarbonates, poly(N-vinyl)-pyrrolidone, polyesteramides, glycolated polyesters, polyphosphoesters, polyphosphazenes, poly[p-carboxyphenoxy]propane], polyhydroxypentanoic acid, soft polyurethanes, polyurethanes having amino acid residues in the backbone, fibrinogen, starch, protein based polymers, polyamino acids, synthetic polyamino acids, zein, modified zein, polyhydroxyalkanoates, pectic acid, actinic acid, modified and non modified fibrin and casein, carboxymethylsulfate, heparansulfates, heparin, chondroitinesulfate, copolymers with PEG and polypropyleneglycol, gummi arabicum, guar, gelatine, lipids and lipoids, polymerizable oils having a low degree of cross-linking, modifications, grafted polymers, and copolymers and/or mixtures of the aforementioned substances.

In some embodiments, the size of the polymer moiety is between approximately 1 KDa to 5000 KDa.

In some embodiments, the siRNA is conjugated to the outer surface of the polymer. Such embodiments are not limited to a specific manner of conjugation. In some embodiments, the conjugation is via a reduction sensitive linkage (e.g., pyridyldithiol propionate-thiol linkage). In some embodiments, the conjugation is via a reduction insensitive linkage (e.g., maleimide-thiol linkage).

Such embodiments are not limited to a particular type or kind of siRNA. In some embodiments, the siRNA is capable of inhibiting a target gene by RNA interference, wherein the siRNA comprises two RNA strands that are complementary to each other. In some embodiments, the siRNA is modified with cholesterol at the 3′ sense strand.

In certain embodiments, the present invention provides polymer moeities comprising a polymer associated with (e.g., complexed, conjugated, encapsulated, absorbed, adsorbed, admixed) an αGalCer glycolipid.

Such embodiments are not limited to a specific type of polymer comprised within the polymer moiety. Indeed, any type or kind of polymer would find use within various embodiments of the present invention. Examples of such polymer moieties include, but are not limited to, poly(L-glutamic acid (PGA), polyethyleneimine (PEI), poly(gamma-glutamic acid), polyglycolic acid, polyglycolic acid trimethyl-carbonates, polyglycolic acidtrimethyl-carbonates, polyglycolides, copolymers of the polylactides and polyglycolides, polylactic acid (PLLA), poly(lactic-co-glycolic acid) poly(D,L lactic-co-glycolide) (PLGA), PLGA derivatives, polyacrylic acid, polyethylene glycol (PEG), PEG derivatives, methoxy polyethylene glycol (mPEG), poly(D,L-lactide)-block-methoxy polyethylene glycol (diblock), polyethyleneoxide-propyleneoxide, polyethylene oxide (PEO), polyether esters (e.g., polyethyleneoxide, polyalkeneoxalates, polyorthoesters as well as their copolymers), poly-L-lysine, chitosan, hyaluronic acid, fatty acids, fatty acyls, polyamine, polyvinyl alcohol, polyvinylalcoholes, polyvinyl pyrrolidinone, acrylic acid ethylene co-polymer, poly(polypropylacrylic acid) (PPAA), polyacrylic acid, methoxy polyethylene, albumin, human serum albumin, cellulose derivatives, cyclodextrins and their derivatives (e.g., including polytryptophan, polytyrosine, polypropyleneoxide), folate linked cyclodextrin, folate linked cyclodextran, β-cyclodextrines, alginate, carrageenan, pectin, dextran, collagen, collagen-N-hydroxysuccinimide, polyaniline, polyalanine, polytryptophan, polytyrosine, polyamino acids, amino sugars, glycerolipid, glycerophospholipid, sphinglipid, sterol lipid, prenol lipid, saccarolipid, polyketide, glucosamines, lipopolysaccarides, aminopolysaccarides, poly(allylamines), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(ethyleneimine), poly(beta-aminoester), poly(hydroxyethyl methacrylate), polyacrylamide, polyethyloxazole, polysaccharides (e.g., dextran), linear and branched polyethylenimine, polyamidoamine, polypropylenimine, polyallylamine, spermine, polyvalerolactones, poly-ε-decalactones, polylactides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides (e.g., polymaleic anhydrides), polyhydroxymethacrylates, fibrin, polycyanoacrylates, polycaprolactonedimethylacrylates, poly-β-maleic acid, polycaprolactonebutyl-acrylates, multiblock polymers (e.g., oligocaprolactonedioles and oligodioxanonedioles), polyetherester multiblock polymers (e.g., PEG and poly(butyleneterephtalates)), polypivotolactones, polycaprolactone-glycolides, poly(g-ethylglutamate), poly(DTH-iminocarbonate), poly(DTE-co-DT-carbonate), poly(bisphenol-A-iminocarbonate), polyorthoesters, polytrimethylcarbonates, polyiminocarbonates, poly(N-vinyl)-pyrrolidone, polyesteramides, glycolated polyesters, polyphosphoesters, polyphosphazenes, poly[p-carboxyphenoxy]propane], polyhydroxypentanoic acid, soft polyurethanes, polyurethanes having amino acid residues in the backbone, fibrinogen, starch, protein based polymers, polyamino acids, synthetic polyamino acids, zein, modified zein, polyhydroxyalkanoates, pectic acid, actinic acid, modified and non modified fibrin and casein, carboxymethylsulfate, heparansulfates, heparin, chondroitinesulfate, copolymers with PEG and polypropyleneglycol, gummi arabicum, guar, gelatine, lipids and lipoids, polymerizable oils having a low degree of cross-linking, modifications, grafted polymers, and copolymers and/or mixtures of the aforementioned substances.

In some embodiments, the αGalCer glycolipid is conjugated to the outer surface of the polymer. Such embodiments are not limited to a specific manner of conjugation. In some embodiments, the conjugation is via a reduction sensitive linkage (e.g., pyridyldithiol propionate-thiol linkage). In some embodiments, the conjugation is via a reduction insensitive linkage (e.g., maleimide-thiol linkage).

In some embodiments, the size of the polymer moiety is between approximately 1 KDa to 5000 KDa.

In certain embodiments, the present invention provides methods for treating conditions, disorders and/or diseases with such compositions comprising associated with a polymer moiety as described herein. The present invention is not limited to specific types of conditions, disorders and/or diseases.

In certain embodiments, compositions comprising a polymer associated with a biomacromolecule agent are further associated with one or more of the following: an additional composition comprising a polymer associated with an antigen (e.g., any antigen described herein); an additional composition comprising a polymer associated with an adjuvant (e.g., any adjuvant described herein); an additional composition comprising a polymer associated with a biomacromolecule agent (e.g., any biomacromolecule agent described herein); a polymer (e.g., any polymer described herein); a complexing agent (e.g., any complexing agent described herein); a crosslinking agent (e.g., any crosslinking agent described herein); and any agent capable of nanoparticle formation upon association with the compositions comprising a polymer associated with a biomacromolecule agent.

Additional embodiments will be apparent to persons skilled in the relevant art based on the teachings contained herein.

### BRIEF DESCRIPTION OF THE DRAWINGS

FIG. 1: Gel permeation chromatogramns of PGA and PGA-peptide conjugates. Red: PGA₁₂₀ₖ, blue: PGA₁₂₀ₖ-MAL-CSSSIINFEKL (1% modification), green: PGA₁₂₀ₖ-SS-CSSSIINFEKL (1% modification).

FIG. 2: PGA-peptide conjugates plus CpG activate BMDCs in vitro. BMDCs were incubated with PGA₁₂₀ₖ-peptide conjugates (1% modification, 2 nmol SIINFEKL/ml) w/or w/o 0.5 μg/ml CpG for 24 h. Expression of DC co-stimulatory markers including CD86 and CD40 was measured by flow cytometry.

FIG. 3: PGA-peptide conjugates plus CpG elicit antigen cross-presentation in vitro. (A) BMDCs were incubated with soluble SIINFEKL or PGA₁₂ₖ-SIINFEKL conjugates (1% modification, 2 nmol SIINFEKL/ml) w/or w/o 0.5 μg/ml CpG for 24 h. Presentation of SIINFEKL on DCs was measured by flow cytometry. (B) BMDCs were incubated with soluble SIINFEKL or PGA₁₂₀ₖ-SIINFEKL conjugates (1% modification, 0.5 nmol SIINFEKL/ml) w/or w/o 0.5 μg/ml CpG for 24 h, followed by co-culture with CFSE-labeled OT-I T cells for 3 days. T-cell proliferation indicated by CFSE dilutions was measured by flow cytometry.

FIG. 4: PGA-peptide conjugates improves LN draining of antigen peptides. C57BL/6 mice were subcutaneously injected with soluble CSSSIINFEKL-FITC, or PGA-SS-CSSSIINFEKL-FITC (1% modification, 30 kD or 120 kD, 20 nmol peptide/mouse) at two sides of tail base. Inguinal and axillary LNs were excised for measurement of fluorescence intensity ex vivo by IVIS at 6 h after injection.

FIG. 5: PGA-peptide conjugates plus CpG elicit antigen-specific T-cell immune responses in vivo. (A) C57BL/6 mice (n=3) were subcutaneously immunized with PBS or PGA₁₂₀ₖ-SS-CSSSIINFEKL (1% modification, 40 nmol peptide/mouse)+CpG (50 μg/mouse) for four times every two weeks. (B) C57BL/6 mice (n=5) were subcutaneously immunized with PBS, soluble ASMTNMELM peptide (20 nmol/mouse)+CpG (50 μg/mouse), or PGA₁₂₀ₖ-MAL-CSSASMTNMELM (5% modification, 20 nmol peptide/mouse)+CpG (50 μg/mouse) for four times weekly. Elicitation of antigen-specific CD8⁺ T cells after each dose was quantified by tetramer staining assays.

FIG. 6: PGA-neo-antigen conjugates+CpG protect animals against tumor growth in vivo. C57BL/6 mice (n=5) were immunized (sc at tail base) with PBS, soluble peptide, or PGA-MAL-CSSASMTNMELM (120 kD, ˜5% modification, 20 nmol peptide/mouse) plus CpG (50 ug/mouse) for 4 times at 1 wk interval and challenged with 0.1 million MC-38 cells, followed by monitoring tumor growth.

FIG. 7: Intratumoral delivery of PGA-peptide conjugates plus CpG eliminates MC38 tumors. C57BL/6 mice were subcutaneously inoculated with 5×10⁵ MC38 cells on day 0, left untreated or treated with a single intratumoral dose of ASMTNMELM+CpG, or PGA₁₂₀ₖ-SS-CSSASMTNMELM (1% molar modification) plus CpG on day 9. Doses of peptide and CpG were 10 ug and 15 ug, respectively. (A) Peripheral blood was collected on days 16, 23, and 30 for tetramer staining assay. (B) Average tumor growth curve. (C) Survival curve. Data show mean±SEM, n=5 (see, Example II).

FIG. 8: A scheme of PEI-SS-CSSASMTNMELM synthesis using SPDP cross-linker (see, Example III).

FIG. 9: GPC analysis of PEI-SS-CSSASMTNMELM conjugate (see, Example III).

FIG. 10: BMDC experiments. (A) Cytotoxicity after 24 h incubation. (B) IL-12p70 secretion after 2 h incubation (see, Example III).

FIG. 11: Tetramer staining after three doses of tail-base sc injection to naïve mice (see, Example III).

FIG. 12: Tetramer staining after the first dose of IT injection to MC38 tumor-bearing mice (see, Example III).

FIG. 13: Tumor volume (A) and survival (B) of MC38 tumor-bearing mice (see, Example III).

### DEFINITIONS

To facilitate an understanding of the present invention, a number of terms and phrases are defined below:

As used here, the term “lipids” refer to fatty substances that are insoluble in water and include fats, oils, waxes, and related compounds. They may be either made in the blood (endogenous) or ingested in the diet (exogenous). Lipids are essential for normal body function and whether produced from an exogenous or endogenous source, they must be transported and then released for use by the cells. The production, transportation and release of lipids for use by the cells is referred to as lipid metabolism. While there are several classes of lipids, two major classes are cholesterol and triglycerides. Cholesterol may be ingested in the diet and manufactured by the cells of most organs and tissues in the body, primarily in the liver. Cholesterol can be found in its free form or, more often, combined with fatty acids as what is called cholesterol esters.

As used herein, the term “complexed” as used herein relates to the non-covalent interaction of a biomacromolecule agent (e.g., antigen, adjuvant, etc) with a polymer.

As used herein, the term “conjugated” as used herein indicates a covalent bond association between a a biomacromolecule agent (e.g., antigen, adjuvant, etc) and a polymer.

As used herein, the term “encapsulated” refers to the location of a biomacromolecule agent (e.g., antigen, adjuvant, etc) that is enclosed or completely contained within the inside of a polymer.

As used herein, the term “absorbed” refers to a biomacromolecule agent (e.g., antigen, adjuvant, etc) that is taken into and stably retained in the interior, that is, internal to the outer surface, of a polymer.

As used herein, the term “adsorbed” refers to the attachment of a biomacromolecule agent (e.g., antigen, adjuvant, etc) to the external surface of a polymer. Such adsorption preferably occurs by electrostatic attraction. Electrostatic attraction is the attraction or bonding generated between two or more oppositely charged or ionic chemical groups. Generally, the adsorption is typically reversible.

As used herein, the term “admixed” refers to a biomacromolecule agent (e.g., antigen, adjuvant, etc) that is dissolved, dispersed, or suspended in a polymer. In some cases, the biomacromolecule agent may be uniformly admixed in the polymer.

As used herein, the terms “biological biomacromolecule” or “biomacromolecule” or “biomacromolecule agent” as used herein refer to a molecule with a molecular mass exceeding 1 kDa which can be isolated from an organism or from cellular culture, e.g., eukaryotic (e.g., mammalian) cell culture or prokaryotic (e.g., bacterial) cell culture. In some embodiments, the use of the term refers to polymers, e.g., biopolymers such as nucleic acids (including, but not limited to, RNA, siRNA, microRNA, interference RNA, mRNA, replicon mRNA, RNA-analogues, DNA, and DNA analogues, etc.), polypeptides (such as proteins), carbohydrates, and lipids. In some embodiments, the term “biomacromolecule” refers to a protein. In some embodiments, the term “biomacromolecule” refers to a recombinant protein or a fusion protein. In some embodiments, the protein is soluble. In some embodiments, the biomacromolecule is an antibody, e.g., a monoclonal antibody. In some embodiments, the biomacromolecule is an adjuvant, an antigen, a therapeutic agent, an imaging agent, etc.

As used herein, the term “antigen” is defined herein as a molecule which contains one or more epitopes that will stimulate a hosts immune system to make a cellular antigen-specific immune response, and/or a humoral antibody response. Antigens can be peptides, proteins, polysaccharides, saccharides, lipids, nucleic acids, and combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components. An antigen may be an oligonucleotide or polynucleotide which expresses an antigen. Antigens can be natural or synthetic antigens, for example, haptens, polyepitopes, flanking epitopes, and other recombinant or synthetically derived antigens (see, e.g., Bergmann, et al., Eur. J. Immunol., 23:2777-2781 (1993); Bergmann, et al., J. Immunol., 157:3242-3249 (1996); Suhrbier, Immunol. and Cell Biol., 75:402-408 (1997)).

As used herein, the term “neo-antigen” or “neo-antigenic” means a class of tumor antigens that arises from a tumor-specific mutation(s) which alters the amino acid sequence of genome encoded proteins.

As used herein, the term “tumor-specific antigen” is defined herein as an antigen that is unique to tumor cells and does not occur in or on other cells in the body.

As used herein, the term “tumor-associated antigen” is defined herein as an antigen that is not unique to a tumor cell and is also expressed in or on a normal cell under conditions that fail to induce an immune response to the antigen.

As used herein, the term “adjuvant” is defined herein as a substance increasing the immune response to other antigens when administered with other antigens. Adjuvants are also referred to herein as “immune potentiators” and “immune modulators”.

As used herein, the term “antigen-presenting cells” are defined herein as highly specialized cells that can process antigens and display their peptide fragments on the cell surface together with molecules required for lymphocyte activation. The major antigen-presenting cells for T cells are dendritic cells, macrophages and B cells. The major antigen-presenting cells for B cells are follicular dendritic cells.

As used herein, the term “cross-presentation” is defined herein as the ability of antigen-presenting cells to take up, process and present extracellular antigens with MHC class I molecules to CD8 T cells (cytotoxic T cells). This process induces cellular immunity against most tumors and against viruses that do not infect antigen-presenting cells. Cross-presentation is also required for induction of cytotoxic immunity by vaccination with protein antigens, for example in tumor vaccination.

As used herein, the terms “immunologic”, “immunological” or “immune” response is the development of a humoral and/or a cellular response directed against an antigen.

As used herein, the term “kit” refers to any delivery system for delivering materials. In the context of the polymer moieites as described herein (e.g., compositions comprising a polymer moiety comprising a polymer associated with a biomacromolecule agent, such delivery systems include systems that allow for the storage, transport, or delivery of such compositions and/or supporting materials (e.g., written instructions for using the materials, etc.) from one location to another. For example, kits include one or more enclosures (e.g., boxes) containing the necessary agents and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising a polymer moiety or the ingredients necessary to synthesize such a polymer moiety, while a second container contains a second agent (e.g., siRNA, an antigen, an adjuvant) (e.g., an antibiotic or spray applicator). Indeed, any delivery system comprising two or more separate containers that each contains a subportion of the total kit components are included in the term “fragmented kit.” In contrast, a “combined kit” refers to a delivery system containing all of the components necessary to synthesize and utilize any of the polymer moieties as described (e.g., in a single box housing each of the desired components). The term “kit” includes both fragmented and combined kits.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “drug” or “therapeutic agent” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “solvent” refers to a medium in which a reaction is conducted. Solvents may be liquid but are not limited to liquid form. Solvent categories include but are not limited to nonpolar, polar, protic, and aprotic.

### DETAILED DESCRIPTION OF THE INVENTION

The present invention relates to polymer moieties comprising polymers associated with biomacromolecule agents configured for treating, preventing or ameliorating various types of disorders, methods of synthesizing the same, and systems and methods utilizing such polymer moeities (e.g., in diagnostic and/or therapeutic settings).

The present invention addresses the need for improved stable and targeted delivery (e.g., in vitro or in vivo) of biomacromolcules (e.g., peptides, nucleic acids, glycolipids). Indeed, the present invention addresses such needs through providing polymer moieties comprising a polymer associated with one or more biomacromolecule agent for stable and targeted delivery of such biomacromolecules, including peptides, nucleic acids, and glycolipids.

### Biomacromolecule Delivery

In certain embodiments, the present invention provides polymer moieties comprising a polymer associated with (e.g., complexed, conjugated, encapsulated, absorbed, adsorbed, admixed) any kind of biomacromolecule agent (e.g., nucleic acid, peptides, glycolipids, etc.).

Such embodiments are not limited to a specific type of polymer comprised within the polymer moiety. Indeed, any type or kind of polymer would find use within various embodiments of the present invention. Examples of such polymer moieties include, but are not limited to, poly(L-glutamic acid (PGA), polyethyleneimine (PEI), poly(gamma-glutamic acid), polyglycolic acid, polyglycolic acid trimethyl-carbonates, polyglycolic acidtrimethyl-carbonates, polyglycolides, copolymers of the polylactides and polyglycolides, polylactic acid (PLLA), poly(lactic-co-glycolic acid) poly(D,L lactic-co-glycolide) (PLGA), PLGA derivatives, polyacrylic acid, polyethylene glycol (PEG), PEG derivatives, methoxy polyethylene glycol (mPEG), poly(D,L-lactide)-block-methoxypolyethylene glycol (diblock), polyethyleneoxide-propyleneoxide, polyethylene oxide (PEO), polyether esters (e.g., polyethyleneoxide, polyalkeneoxalates, polyorthoesters as well as their copolymers), poly-L-lysine, chitosan, hyaluronic acid, fatty acids, fatty acyls, polyamine, polyvinyl alcohol, polyvinylalcoholes, polyvinyl pyrrolidone, acrylic acid ethylene co-polymer, poly(polypropylacrylic acid) (PPAA), polyacrylic acid, methoxy polyethylene, albumin, human serum albumin, cellulose derivatives, cyclodextrins and their derivatives (e.g., including polytryptophan, polytyrosine, polypropyleneoxide), folate linked cyclodextrin, folate linked cyclodextran, f-cyclodextrines, alginate, carrageenan, pectin, dextran, collagen, collagen-N-hydroxysuccinimide, polyaniline, polyalanine, polytryptophan, polytyrosine, polyamino acids, amino sugars, glycerolipid, glycerophospholipid, sphinglipid, sterol lipid, prenol lipid, saccarolipid, polyketide, glucosamines, lipopolysaccarides, aminopolysaccarides, poly(allylamines), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(ethyleneimine), poly(beta-aminoester), poly(hydroxyethyl methacrylate), polyacrylamide, polyethyloxazole, polysaccharides (e.g., dextran), linear and branched polyethylenimine, polyamidoamine, polypropylenimine, polyallylamine, spermine, polyvalerolactones, poly-ε-decalactones, polylactides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides (e.g., polymaleic anhydrides), polyhydroxymethacrylates, fibrin, polycyanoacrylates, polycaprolactonedimethylacrylates, poly-β-maleic acid, polycaprolactonebutyl-acrylates, multiblock polymers (e.g., oligocaprolactonedioles and oligodioxanonedioles), polyetherester multiblock polymers (e.g., PEG and poly(butyleneterephtalates)), polypivotolactones, polycaprolactone-glycolides, poly(g-ethylglutamate), poly(DTH-iminocarbonate), poly(DTE-co-DT-carbonate), poly(bisphenol-A-iminocarbonate), polyorthoesters, polytrimethylcarbonates, polyiminocarbonates, poly(N-vinyl)-pyrrolidone, polyesteramides, glycolated polyesters, polyphosphoesters, polyphosphazenes, poly[p-carboxyphenoxy]propane], polyhydroxypentanoic acid, soft polyurethanes, polyurethanes having amino acid residues in the backbone, fibrinogen, starch, protein based polymers, polyamino acids, synthetic polyamino acids, zein, modified zein, polyhydroxyalkanoates, pectic acid, actinic acid, modified and non modified fibrin and casein, carboxymethylsulfate, heparansulfates, heparin, chondroitinesulfate, copolymers with PEG and polypropyleneglycol, gummi arabicum, guar, gelatine, lipids and lipoids, polymerizable oils having a low degree of cross-linking, modifications, grafted polymers, and copolymers and/or mixtures of the aforementioned substances.

In some embodiments, the biomacromolecule agent is conjugated to the outer surface of the polymer. In some embodiments, the conjugation is via a reduction sensitive linkage (e.g., pyridyldithiol propionate-thiol linkage). In some embodiments, the conjugation is via a reduction insensitive linkage (e.g., maleimide-thiol linkage).

In some embodiments, the size of the polymer moiety is between approximately 1 KDa to 5000 KDa.

In some embodiments, the biomacromolecule agent is a peptide.

For example, in some embodiments, the peptide is an antigen. In some embodiments wherein the peptide is an antigen, the composition further comprises an adjuvant (as described herein).

As noted, the peptide is not limited to a particular type of peptide.

In some embodiments, the peptide is Adrenocorticotropic Hormone (ACTH), a growth hormone peptide, a Melanocyte Stimulating Hormone (MSH), Oxytocin, Vasopressin, Corticotropin Releasing Factor (CRF), a CRF-related peptide, a Gonadotropin Releasing Hormone Associated Peptide (GAP), Growth Hormone Releasing Factor (GRF), Lutenizing Hormone Release Hormone (LH-RH), an orexin, a Prolactin Releasing Peptide (PRP), a somatostatin, Thyrotropin Releasing Hormone (THR), a THR analog, Calcitonin (CT), a CT-precursor peptide, a Calcitonin Gene Related Peptide (CGRP), a Parathyroid Hormone (PTH), a Parathyroid Hormone Related Protein (PTHrP), Amylin, Glucagon, Insulin, an Insulin-like peptide, NeuroPeptide Y (NPY), a Pancreatic Polypeptide (PP), Peptide YY (PYY), Cholecystokinin (CCK), a CCK-related peptide, Gastrin Releasing Peptide (GRP), Gastrin, a Gastrin-related peptide, a Gastrin inhibitory peptide, Motilin, Secretin, Vasoactive Intestinal Peptide (VIP), a VIP-related peptide, an Atrial-Natriuretic Peptide (ANP), a Brain Natriuretic Peptide (BNP), a C-Type Natriuretic Peptide (CNP), a tachykinin, an angiotensin, a renin substrate, a renin inhibitor, an endothelin, an endothelin-related peptide, an opioid peptide, a thymic peptide, an adrenomedullin peptide, an allostatin peptide, an amyloid beta-protein fragment, an antimicrobial peptide, an antioxidant peptide, an apoptosis related peptide, a Bag Cell Peptide (BCPs), Bombesin, a bone Gla protein peptide, a Cocaine and Amphetamine Related Transcript (CART) peptide, a cell adhesion peptide, a chemotactic peptide, a complement inhibitor, a cortistatin peptide, a fibronectin fragment, a fibrin related peptide, FMRF, a FMRF amide-related peptide (FaRP), Galanin, a Galanin-related peptide, a growth factor, a growth factor-related peptide, a G-Therapeutic Peptide-Binding Protein fragment, Gualylin, Uroguanylin, an Inhibin peptide, Interleukin (IL), an Interleukin Receptor protein, a laminin fragment, a leptin fragment peptide, a leucokinin, Pituitary Adenylate Cyclase Activating Polypeptide (PAPCAP), Pancreastatin, a polypeptide repetitive chain, a signal transducing reagent, a thrombin inhibitor, a toxin, a trypsin inhibitor, a virus-related peptide, an adjuvant peptide analog, Alpha Mating Factor, Antiarrhythmic Peptide, Anorexigenic Peptide, Alpha-1 Antitrypsin, Bovine Pineal Antireproductive Peptide, Bursin, C3 Peptide P16, Cadherin Peptide, Chromogranin A Fragment, Contraceptive Tetrapeptide, Conantokin G, Conantokin T, Crustacean Cardioactive Peptide, C-Telopeptide, Cytochrome b588 Peptide, Decorsin, Delicious Peptide, Delta-Sleep-Inducing Peptide, Diazempam-Binding Inhibitor Fragment, Nitric Oxide Synthase Blocking Peptide, OVA Peptide, Platelet Calpain Inhibitor (P1), Plasminogen Activator Inhibitor 1, Rigin, Schizophrenia Related Peptide, Sodium Potassium Atherapeutic Peptidase Inhibitor-1, Speract, Sperm Activating Peptide, Systemin, a Thrombin receptor agonist, Tuftsin, Adipokinetic Hormone, Uremic Pentapeptide, Antifreeze Polypeptide, Tumor Necrosis Factor (TNF), Leech [Des Asp10]Decorsin, L-Ornithyltaurine Hydrochloride, P-Aminophenylacetyl Tuftsin, Ac-Glu-Glu-Val-Val-Ala-Cys-pNA, Ac-Ser-Asp-Lys-Pro, Ac-rfwink-NH2, Cys-Gly-Tyr-Gly-Pro-Lys-Lys-Lys-Arg-Lys-Val-Gly-Gly, D-Ala-Leu, D-D-D-D-D, D-D-D-D-D-D, N-P-N-A-N-P-N-A, V-A-I-T-V-L-V-K, V-G-V-R-V-R, V-I-H-S, V-P-D-P-R, Val-Thr-Cys-Gly, R-S-R, Sea Urchin Sperm Activating Peptide, a SHU-9119 antagonist, a MC3-R antagonist, a MC4-R antagonist, Glaspimod, HP-228, Alpha 2-Plasmin Inhibitor, APC Tumor Suppressor, Early Pregnancy Factor, Gamma Interferon, Glandular Kallikrei N-1, Placental Ribonuclease Inhibitor, Sarcolecin Binding Protein, Surfactant Protein D, Wilms' Tumor Suppressor, GABAB 1b Receptor Peptide, Prion Related Peptide (iPRP13), Choline Binding Protein Fragment, Telomerase Inhibitor, Cardiostatin Peptide, Endostatin Derived Peptide, Prion Inhibiting Peptide, N-Methyl D-Aspartate Receptor Antagonist, C-Peptide-Analog, myelin basic protein (MBP), myelin oligodendrocyte protein (MOG), and proteolipid protein (PLP), insulin, and their mimotopes.

In some embodiments, the peptide is selected from 177Lu-DOTA0-Tyr3-Octreotate, Abarelix acetate, ADH-1, Afamelanotidec, melanotan-1, CUV1647, Albiglutide, Aprotinin, Argipressin, Atosiban acetate, Bacitracin, Bentiromide, a BH3 domain, Bivalirudin, Bivalirudin trifluoroacetate hydrate, Blisibimod, Bortezomib, Buserelin, Buserelin acetate, Calcitonin, Carbetocin, Carbetocin acetate, Cecropin A and B, Ceruletide, Ceruletide diethylamine, Cetrorelix, Cetrorelix acetate, Ciclosporine, Cilengitidec, EMD121974, Corticorelin acetate injection, hCRF, Corticorelin ovine triflutate, corticorelin trifluoroacetate, Corticotropin, Cosyntropin, ACTH 1-24, tetracosactide hexaacetate, Dalbavancin, Daptomycin, Degarelix acetate, Depreotide trifluoroacetate (plus sodium pertechnetate), Desmopressin acetate, Desmopressin DDAVP, Dulaglutide, Ecallantide, Edotreotide (plus yttrium-90), Elcatonin acetate, Enalapril maleate (or 2-butanedioate), Enfuvirtide, Eptifibatide, Exenatide, Ganirelix acetate, Glatiramer acetate, Glutathion, Gonadorelin, Gonadorelin acetate, GnRH, LHRH, Goserelin, Goserelin acetate, Gramicidin, Histrelin acetate, Human calcitonin, Icatibant, Icatibant acetate, IM862, oglufanide disodium, KLAKLAK, Lanreotide acetate, Lepirudin, Leuprolide, Leuprolide acetate, leuprorelin, Liraglutide, Lisinopril, Lixisenatide, Lypressin, Magainin2, MALP-2Sc, macrophage-activating lipopeptide-2 synthetic, Nafarelin acetate, Nesiritide, NGR-hTNF, Octreotide acetate, Oritavancin, Oxytocin, Pasireotide, Peginesatide, Pentagastrin, Pentetreotide (plus indium-111), Phenypressin, Pleurocidin, Pramlintide, Protirelin, thyroliberin, TRH, TRF, Salmon calcitonin, Saralasin acetate, Secretin (human), Secretin (porcine), Semaglutide, Seractide acetate, ACTH, corticotropin, Sermorelin acetate, GRF 1-29, Sinapultide, KL4 in lucinactant, Sincalide, Somatorelin acetate, GHRH, GHRF, GRF, Somatostatin acetate, Spaglumat magnesium (or sodium) salt, Substance P, Taltirelin hydrate, Teduglutide, Teicoplanin. Telavancin, Teriparatide, Terlipressin acetate, Tetracosactide, Thymalfasin, thymosin a-1, Thymopentin, Trebananib, Triptorelin, Triptorelin pamoate, Tyroserleutide, Ularitide, Vancomycin, Vapreotide acetate, Vasoactive intestinal peptide acetate, Vx-001c, TERT572Y, Ziconotide acetate, a5-a6 Bax peptide, and D-defensin.

In some embodiments, the peptide is any peptide which would assist in achieving a desired purpose with the composition. For example, in some embodiments, the peptide is any peptide that will facilitate treatment of any type of disease and/or disorder (e.g., peripheral ischemia, cancer, inflammatory disorders, genetic disorders, etc.).

In some embodiments, the biomacromolecule agent is a nucleic acid. Such embodiments encompass any type of nucleic acid molecule including, but not limited to, RNA, siRNA, microRNA, interference RNA, mRNA, replicon mRNA, RNA-analogues, DNA, and DNA analogues. In certain embodiments, the present invention provides methods for treating conditions, disorders and/or diseases with such polymer moieties.

The present invention is not limited to specific types of conditions, disorders and/or diseases.

In some embodiments, the condition, disorder and/or disease is peripheral ischemia, cancer, an inflammatory disorder, a genetic disorder, etc.

In some embodiments, the condition, disorder and/or disease is selected from erythropoietic porphyries, T2 diabetes, antifibrinolytic, central diabetes insipidus, delaying the birth in case of threat of premature birth, antibiotic, cystic fibrosis, angina, anticoagulant in patients with unstable angina undergoing PTCA or PCI, systemic lupus erythematosus, hypercalcemia, osteoporosis, pagets disease, carbetocin works as an oxytocic, antihemorrhagic and uterotonic drug in the peripheral nervous system, prevention of uterine atony, induction, and control postpartum bleeding or haemorrhage, stimulant of the gastric secretion, for treat hormone-sensitive cancers of the prostate and breast, inhibition of premature LH surges in women undergoing controlled ovarian stimulation, immunosuppression in organ transplantation to prevent rejection, peritumoral brain edema, diagnosis of ACTHdependent Cushing's syndrome, allergies, ankylosing spondylitis, psoriasis, chorioditis, erythema, keratitis, sclerosis, dermatomyositis, rheumatoid arthritis, Stevens-Johnson Syndrome, ulcerative colitis, diagnosis of adrenocortical insufficiency, antibiotic, systemic infections caused by gram positive organisms, nocturnal enuresis, nocturia, and stoppage of bleeding or haemorrhage in haemophilia A patients, acute hereditary angioderma, postmenopausal osteoporosis, anti-parathyroid, Paget's disease, hypercalcaemia, hypertension, AIDS/HIV-1 infection, acute coronary syndrome, unstable angina undergoing PCI, Alzheimer's and Parkinson's disease, inhibition of premature LH surges in women undergoing controlled ovarian hyperstimulation, Relapsing-Remitting Multiple Sclerosis, hepatic insufficiency, wound healing, inflammation of respiratory tract, asthenia, release of follicle-stimulating hormone (FSH) and luteinizing hormone (LH) from the anterior pituitary, stimulate the secretion of gonadotropin during disturbances fertility, and diagnosis of the functional capacity and response of the gonadotropes of the anterior pituitary, for skin lesions, surface wounds and eye infections, postmenopausal osteoporosis, Paget's disease, hypercalcaemia, hereditary angioedema, immune system related diseases, acromegaly, anticoagulant, fibroids and endometriosis, central diabetes insipidus, Cushing's syndrome, diabetic foot ulcers, treatment of central precocious puberty, uterine fibriods and endometriosis, vasodilatory, natriuretic, diuretic and neurohormonal effects, acromegaly, carcinoid syndrome, acute bacterial skin and skin structure infections, initiation or improvement of uterine contractions, and control postpartum bleeding or haemorrhage, hematide Chronic kidney disease associated anemia, stomatitis, pharyngitis, diagnostic assessment of thyroid function, postmenopausal osteoporosis, hypercalcaemia, diagnosis of pancreatic exocrine dysfunction, and gastrinoma, Zollinger-Ellison syndrome, prevention of RDS in premature infants, and meconium aspiration syndrome, acute variceal bleeding, allergic rhinitis and conjunctivitis, spinocerebellar degeneration/ataxia, Short Bowel Syndrome, antibiotic, bactericidal, teriparatide is the only anabolic (i.e., bone growing) agent indicated for use in postmenopausal women with osteoporosis, Cortrosyn Analogue of adrenocorticotrophic hormone (ACTH) used for diagnostic purposes, treatment of adrenal insufficiency, epilepsia, Chronic hepatitis B, chronic hepatitis C, primary and secondary immune deficiencies, acute decompensated heart failure, colitis, esophageal variceal bleeding in patients with cirrhotic liver disease and AIDS-related diarrhea, sarcoidosis and acute lung injury, and severe chronic pain.

In some embodiments, the following Table 1 recites a peptide and disorder and/or disease to be treated.

In certain embodiments, the biomacromocule agents are conjugated with a polymer within the polymer moiety via a linkage agent (linker). The present invention is not limited to a particular type or kind of linker.

For example, in some embodiments, the conjugation is via a reduction sensitive linkage (e.g., pyridyldithiol propionate-thiol linkage). In some embodiments, the conjugation is via a reduction insensitive linkage (e.g., maleimide-thiol linkage).

In some embodiments, the linker connects the polymer with a therapeutic compound. In some embodiments, the linker is configured such that its decomposition leads to the liberation (e.g., non-reversible liberation) of the therapeutic agent (e.g., at the target site (e.g., site of tumor, CNS, and/or inflammatory site)). The linker may influence multiple characteristics of a polymer moiety including, but not limited to, properties of the therapeutic agent (e.g., stability, pharmacokinetic, organ distribution, bioavailability, and/or enzyme recognition (e.g., when the therapeutic agent (e.g., prodrug)) is enzymatically activated)).

In some embodiments, the linker is an elimination linker.

In some embodiments, elongated analogs (e.g., double spacers) are used as linkage agents (e.g., to decrease steric hindrance (e.g., for large therapeutic agents)).

In some embodiments, a polymer moiety of the present invention comprises an enol based linker (e.g., that undergoes an elimination reaction to release therapeutic agent (e.g., prodrug)).

In some embodiments, the linker is a cyclization based linker.

In some embodiments, a polymer moiety of the present invention comprises a combination of one or more linkers. For example, in some embodiments, a polymer moiety comprises a combination of two or more elimination linkers. In some embodiments, a polymer moiety of the present invention comprises two or more cyclization linkers. In some embodiments, a polymer moiety of the present invention comprises a one or more elimination linkers and one or more cyclization linkers, or a combination of one or more different types of linkers described herein.

In some embodiments, a polymer moiety of the present invention comprises branched self-elimination linkers. Thus, in some embodiments, use of branched linkers provides a conjugate that can present increased concentrations of a therapeutic agent to a target site (e.g., inflammatory site, tumor site, etc.).

In some embodiments, the linker is a trigger agent. The present invention is not limited to a particular trigger agent or to any particular cleavage and/or processing of the trigger agent. In some embodiments, the present invention provides a polymer moiety comprising a trigger agent that is sensitive to (e.g., is cleaved by) hypoxia. The present invention is not limited to particular hypoxia activated trigger agents. In some embodiments, the hypoxia activated trigger agents include, but are not limited to, indoquinones, nitroimidazoles, and nitroheterocycles (see, e.g., Damen, E. W. P., et al., Bioorganic & Medicinal Chemistry, 2002. 10(1): p. 71-77; Hay, M. P., et al., Journal of Medicinal Chemistry, 2003. 46(25): p. 5533-5545; Hay, M. P., et al., Journal of the Chemical Society-Perkin Transactions 1, 1999(19): p. 2759-2770; each herein incorporated by reference in their entireties).

In some embodiments, a polymer moiety of the present invention utilizes a quinone, N-oxide and/or (hetero)aromatic nitro groups. In some embodiments, a heteroaromatic nitro compound present in a polymer moiety of the present invention is reduced to either an amine or a hydroxylamine, thereby triggering the spontaneous release of a therapeutic agent/drug. In some embodiments, the present invention provides therapeutic agents and/or therapeutic agent antagonists coupled to polymer moieties with a linkage agent connected to a trigger agent that degrades upon detection of reduced pO2 concentrations (e.g., through use of a re-dox linker).

In some embodiments, the present invention provides a polymer moiety comprising a trigger agent that is sensitive to (e.g., is cleaved by) and/or that associates with a tumor associated enzyme. In some embodiments, the present invention provides a polymer moeity comprising a trigger that is sensitive to (e.g., is cleaved by) and/or that associates with a glucuronidase.

In some embodiments, the present invention provides a polymer moeity comprising a trigger agent that is sensitive to (e.g., is cleaved by) and/or that associates with brain enzymes. For example, trigger agents such as indolequinone are reduced by brain enzymes such as, for example, diaphorase (see, e.g., Damen, E. W. P., et al., Bioorganic & Medicinal Chemistry, 2002. 10(1): p. 71-77; herein incorporated by reference in its entirety). For example, in such embodiments, the antagonist is only active when released during hypoxia to prevent respiratory failure.

In some embodiments, the present invention provides a polymer moiety comprising a trigger agent that is sensitive to (e.g., is cleaved by) and/or that associates with a protease. The present invention is not limited to any particular protease. In some embodiments, the protease is a cathepsin. In some embodiments, a trigger comprises a Lys-Phe-PABC moiety (e.g., that acts as a trigger). In some embodiments, a Lys-Phe-PABC moiety linked to doxorubicin, mitomycin C, and paclitaxel are utilized as a trigger-therapeutic conjugate in a polymer moiety provided herein (e.g., that serve as substrates for lysosomal cathepsin B or other proteases expressed (e.g., overexpressed) in tumor cells. In some embodiments, utilization of a 1,6-elimination spacer/linker is utilized (e.g., to permit release of therapeutic drug post activation of trigger).

In some embodiments, the present invention provides a polymer moiety comprising a trigger agent that is sensitive to (e.g., is cleaved by) and/or that associates with plasmin. The serine protease plasmin is over expressed in many human tumor tissues. Tripeptide specifiers (e.g., including, but not limited to, Val-Leu-Lys) have been identified and linked to anticancer drugs through elimination or cyclization linkers.

In some embodiments, the present invention provides a polymer moiety comprising a trigger agent that is sensitive to (e.g., is cleaved by) and/or that associates with a matrix metalloproteases (MMPs). In some embodiments, the present invention provides a polymer moiety comprising a trigger that is sensitive to (e.g., is cleaved by) and/or that associates with β-Lactamase (e.g., a β-Lactamase activated cephalosporin-based prodrug).

In some embodiments, the present invention provides a polymer moiety comprising a trigger agent that is sensitive to (e.g., is cleaved by) and/or activated by a receptor (e.g., expressed on a target cell (e.g., a tumor cell)). Thus, in some embodiments, a polymer moiety comprises a receptor binding motif conjugated to a therapeutic agent (e.g., cytotoxic drug) thereby providing target specificity.

In some embodiments, the present invention provides a polymer moiety comprising a trigger agent that is sensitive to (e.g., is cleaved by) and/or activated by a nucleic acid.

### RNA Interference

In certain embodiments, the polymer moeities are used within RNA interference methods and systems.

RNA interference is a highly conserved mechanism triggered by double-stranded RNA (dsRNA) and able to down regulate transcript of genes homologous to the dsRNA. The dsRNA is first processed by Dicer into short duplexes of 21-23 nt, called short interfering RNAs (siRNAs). Incorporated in RNA-induced silencing complex (RISC), they are able to mediate gene silencing through cleavage of the target mRNA. “siRNA” or “small-interfering ribonucleic acid” refers to two strands of ribonucleotides which hybridize along a complementary region under physiological conditions. The siRNA molecules comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. Methods to analyze and identify siRNAs with sufficient sequence identity in order to effectively inhibit expression of a specific target sequence are known in the art. A suitable mRNA target region would be the coding region. Also suitable are untranslated regions, such as the 5′-UTR, the 3′-UTR, and splice junctions as long as the regions are unique to the mRNA target and not directed to a mRNA poly A tail.

In some embodiments, siRNA conjugated with polymers (e.g., PGA) are utilized conducting methods and systems involving RNA interference.

Such embodiments are not limited to a particular size or type of siRNA molecule. The length of the region of the siRNA complementary to the target, for example, may be from 15 to 100 nucleotides, 18 to 25 nucleotides, 20 to 23 nucleotides, or more than 15, 16, 17 or 18 nucleotides. Where there are mismatches to the corresponding target region, the length of the complementary region is generally required to be somewhat longer.

In certain embodiments, it is contemplated that the siRNA delivery approach using the polymer moieties disclosed herein (e.g., through conjugation of the siRNA within a polymer (e.g., PGA)) can be used to inhibit any gene of interest.

The present invention is not limited to particular methods for generating polymer moieties comprising polymers conjugated with siRNA molecules. In some embodiments, the siRNA is conjugated to the outer surface of the polymer. Such embodiments are not limited to a specific manner of conjugation. In some embodiments, the conjugation is via a reduction sensitive linkage (e.g., pyridyldithiol propionate-thiol linkage). In some embodiments, the conjugation is via a reduction insensitive linkage (e.g., maleimide-thiol linkage).

Such embodiments are not limited to a particular manner of characterizing the polymer moiety comprising a polymer associated with (e.g., conjugated) siRNA. In some embodiments, the morphology of the polymer moiety is observed by TEM. In some embodiments, the size distribution of polymer moeity is analyzed by dynamic light scattering (DLS) using a Malven Nanosizer instrument and GPC assay.

Such embodiments are not limited to a particular manner of assessing the delivery profile of the siRNA in vitro and in vivo. In some embodiments, labelling the siRNA molecules with an imaging agent (e.g., fluorescent dye Cy3) permits visualization of the biodistribution of siRNA molecules at the organ level and also the intracellular delivery profile. In some embodiments, RT-PCR and western blot are used to analyze the target protein at the mRNA level and protein level, respectively.

As such, in certain embodiments, the present invention provides methods for inhibiting a target gene in a cell comprising introducing into the cell a siRNA capable of inhibiting the target gene by RNA interference, wherein the siRNA comprises two RNA strands that are complementary to each other, wherein the siRNA is conjugated with a polymer (e.g., PGA) within a polymer moiety. In some embodiments, the siRNA is modified with cholesterol at the 3′ sense strand. In some embodiments, the cell is within a human being.

In certain embodiments, polymer moieties comprising polymers associated with (e.g., complexed, conjugated, encapsulated, absorbed, adsorbed, admixed) siRNAs specific for proprotein convertase subtilisin/kexin 9 (PCSK9). Compelling evidence has shown that an elevated plasma level of low-density lipoprotein cholesterol (LDL-C) is a cardinal risk factor for coronary heart disease (CHD) (see, e.g., Law, M. R.; et al., British Medical Journal 2003, 326 (7404), 1423-1427; Boekholdt, S. M.; et al., Jama-Journal of the American Medical Association 2012, 307 (12), 1302-1309; Sniderman, A. D.; et al., Circulation-Cardiovascular Quality and Outcomes 2011, 4 (3), 337-U144). PCSK9 synthesized in the liver performs important roles in regulating LDL-C: PCSK9 can bind to the LDL receptor (LDLR) on hepatocytes and prevent the recycling of LDLR from lysosomes to the cell surface, and this in turn leads to the down-regulation of LDLR and increased levels of LDL-C (see, e.g., Maxwell, K. N.; et al., Proceedings of the National Academy of Sciences of the United States of America 2004, 101 (18), 7100-7105; Dadu, R. T.; et al., Nature Reviews Cardiology 2014, 11 (10), 563-575; Horton, J. D.; et al., Trends in Biochemical Sciences 2007, 32 (2), 71-77). Therefore PCSK9 inhibition can potentially decrease LDL-C (see, e.g., Shen, L.; et al., Pharmacological Research 2013, 73, 27-34). Therapeutic approaches under development for PCSK9 inhibition in vivo include siRNA-mediated knockdown of PCSK9 and vaccination against PCSK9 (see, e.g., Fitzgerald, K.; et al., Lancet 2014, 383 (9911), 60-68; Galabova, G.; et al., Circulation 2013, 128 (22)), but both strategies face the major challenge: how to efficiently deliver therapeutic agents to the target cells, namely hepatocytes and immune cells, respectively, in order to maximize the in vivo efficacy of each strategy.

Such embodiments are not limited to use of a particular PCSK9 siRNA sequence. In some embodiments, the PCSK9 siRNA sequence is cross-reactive to murine, rat, nonhuman primate and human PCSK9 mRNA (see, e.g., Frank-Kamenetsky, et al., Proceedings of the National Academy of Sciences of the U.S. Pat. No. 2,008,105 (33), 11915-11920).

In certain embodiments, the present invention provides methods for inhibiting a PCSK9 gene in a cell comprising introducing into the cell a PCSK9 siRNA capable of inhibiting the PCSK9 gene by RNA interference, wherein the PCSK9 siRNA comprises two RNA strands that are complementary to each other, wherein the PCSK9 siRNA is conjugated with a polymer (e.g., PGA). In some embodiments, the PCSK9 siRNA is modified with cholesterol at the 3′ sense strand. In some embodiments, the cell is within a human being.

In certain embodiments, the present invention provides methods for reducing serum LDL-C levels in patient (e.g., human patient), comprising administering to the patient a therapeutically effective amount of a pharmaceutical composition comprising a PCSK9 siRNA conjugated with a polymer (e.g., PGA), wherein the PCSK9 siRNA is capable of inhibiting the PCSK9 gene by RNA interference, wherein the PCSK9 siRNA comprises two RNA strands that are complementary to each other, wherein inhibiting of the PCSK9 gene results in reduction of serum LDL-C levels.

In certain embodiments, the present invention provides methods for treating coronary heart disease in a patient through reducing serum LDL-C levels in the patient, comprising administering to the patient a therapeutically effective amount of a pharmaceutical composition comprising a PCSK9 siRNA conjugated with a polymer (e.g., PGA), wherein the PCSK9 siRNA is capable of inhibiting the PCSK9 gene by RNA interference, wherein the PCSK9 siRNA comprises two RNA strands that are complementary to each other, wherein inhibiting of the PCSK9 gene results in reduction of serum LDL-C levels.

### Immune Response Stimulation

In certain embodiments, the polymer moieties are used to activate an immune response. Such embodiments are not limited to a particular manner of activating an immune response.

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naïve or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines.

In certain embodiments, polymers associated with (e.g., conjugated) an antigen are used for inducing an immune response. In some embodiments, the polymers are further associated with (e.g., complexed, conjugated, encapsulated, absorbed, adsorbed, admixed) an adjuvant (e.g., dendritic cell targeting molecule (DC)). In some embodiments, the polymers are co-administered with an adjuvant.

Such embodiments are not limited to particular antigen. Indeed, antigens can be peptides, proteins, polysaccharides, saccharides, lipids, glycolipids, nucleic acids, or combinations thereof. The antigen can be derived from any source, including, but not limited to, a virus, bacterium, parasite, plant, protozoan, fungus, tissue or transformed cell such as a cancer or leukemic cell and can be a whole cell or immunogenic component thereof, e.g., cell wall components or molecular components thereof.

In some embodiments, the antigens are known in the art and are available from commercial government and scientific sources. In some embodiments, the antigens are whole inactivated or attenuated organisms. These organisms may be infectious organisms, such as viruses, parasites and bacteria. These organisms may also be tumor cells. The antigens may be purified or partially purified polypeptides derived from tumors or viral or bacterial sources. Criteria for identifying and selecting effective antigenic peptides (e.g., minimal peptide sequences capable of eliciting an immune response) can be found in the art. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

In some embodiments, the antigen is a self antigen. As used herein, the term “self-antigen” refers to an immunogenic antigen or epitope which is native to a mammal and which may be involved in the pathogenesis of an autoimmune disease.

In some embodiments, the antigen is a viral antigen. Viral antigens can be isolated from any virus including, but not limited to, a virus from any of the following viral families: Arenaviridae, Arterivirus, Astroviridae, Baculoviridae, Badnavirus, Barnaviridae, Bimaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picomaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

In some embodiments, the antigen is a bacterial antigen. Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some embodiments, the antigen is a parasite antigen. Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

In some embodiments, the antigen is an allergen and environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hombeam (Carpinus) and olive (Olea), cedar (Cryptomeria and Juniperus), Plane tree (Platanus), the order of Poales including i.e. grasses of the genera Lolium. Phleum. Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

In some embodiments, the antigen is a tumor antigen. The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGS), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), human EGFR protein or its fragments, such as human EGFR residues 306-325 (SCVRACGADSYEMEEDGVRK) and residues 897-915 (VWSYGVTVWELMTFGSKPY), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, TPS, WT1 (and WT1-derivaed peptide sequences: WT1 126-134 (RMFP NAPYL), WT1 122-140 (SGQARMFPNAPYLPSCLES), and WT1 122-144 (SGQARMFPNAPYLPSCLESQPTI)), MUC1 (and MUC1-derived peptides and glycopeptides such as RPAPGS, PPAHGVT, and PDTRP), LMP2, EGFRvIII, Idiotype, GD2, Ras mutant, p53 mutant, Proteinase3 (PR1), Survivin, hTERT, Sarcoma translocation breakpoints, EphA2, EphA4, LMW-PTP, PAP, ML-IAP, AFP, ERG (TMPRSS2 ETS fusion gene), NA17, PAX3, ALK, Androgen receptor, Cyclin B1, Polysialic acid, MYCN, RhoC, TRP-2, GD3, Fucosyl GMI, Mesothelin, sLe (animal), CYP1B1, PLAC1, GM3, BORIS, Tn, GloboH, NY-BR-1, RGS5, SART3, STn, Carbonic anhydrase IX, PAX5, OY-TES1, Sperm protein 17, LCK, HMWMAA, AKAP-4, XAGE 1, B7H3, Legumain, Tie 2, Page4, VEGFR2, MAD-CT-1, FAP, PDGFR-alpha, PDGFR-β, MAD-CT-2, Fos-related antigen 1, ERBB2, Folate receptor 1 (FOLR1 or FBP), IDH1, IDO, LY6K, fins-related tyro-sine kinase 1 (FLT1, best known as VEGFR1), KDR PADRE, TA-CIN (recombinant HPV16 L2E7E6), SOX2, and aldehyde dehydrogenase.

One of the critical barriers to developing curative and tumor-specific immunotherapy is the identification and selection of highly specific and restricted tumor antigens to avoid autoimmunity. Tumor neo-antigens, which arise as a result of genetic change (e.g., inversions, translocations, deletions, missense mutations, splice site mutations, etc.) within malignant cells, represent the most tumor-specific class of antigens.

In some embodiments, the antigen is a neo-antigen. The term neoantigen is used herein to define any newly expressed antigenic determinant. Neoantigens may arise upon conformational change in a protein, as newly expressed determinants (especially on the surfaces of transformed or infected cells), as the result of complex formation of one or more molecules or as the result of cleavage of a molecule with a resultant display of new antigenic determinants. Thus, as used herein, the term neoantigen covers antigens expressed upon infection (e.g. viral infection, protozoal infection or bacterial infection), in prion-mediated diseases, an on cell transformation (cancer), in which latter case the neoantigen may be termed a tumour-associated antigen.

The present invention is not limited to a particular manner of identifying neo-antigens. In some embodiments, identification of neo-antigens involves identifying all, or nearly all, mutations in the neoplasia/tumor at the DNA level using whole genome sequencing, whole exome (e.g., only captured exons) sequencing, or RNA sequencing of tumor versus matched germline samples from each patient. In some embodiments, identification of neo-antigens involves analyzing the identified mutations with one or more peptide-MHC binding prediction algorithms to generate a plurality of candidate neo-antigen T cell epitopes that are expressed within the neoplasia/tumor and may bind patient HLA alleles. In some embodiments, identification of neo-antigens involves synthesizing the plurality of candidate neo-antigen peptides selected from the sets of all neo open reading frame peptides and predicted binding peptides for use in a cancer vaccine.

As such, the present invention is based, at least in part, on the ability to identify all, or nearly all, of the mutations within a neoplasia/tumor (e.g., translocations, inversions, large and small deletions and insertions, missense mutations, splice site mutations, etc.). In particular, these mutations are present in the genome of neoplasia/tumor cells of a subject, but not in normal tissue from the subject. Such mutations are of particular interest if they lead to changes that result in a protein with an altered amino acid sequence that is unique to the patient's neoplasia/tumor (e.g., a neo-antigen). For example, useful mutations may include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence; and the like. Peptides with mutations or mutated polypeptides arising from, for example, splice-site, frameshift, read-through, or gene fusion mutations in tumor cells may be identified by sequencing DNA, RNA or protein in tumor versus normal cells.

Also within the scope of the present invention is personal neo-antigen peptides derived from common tumor driver genes and may further include previously identified tumor specific mutations.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLiD system from Applied BioSystems, and the Heliscope system from Helicos Biosciences. Sequencing-by-synthesis platforms have also been described by Pacific Biosciences and VisiGen Biotechnologies. Each of these platforms can be used in the methods of the invention. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair. Subsequent to the capture, the sequence may be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or in a step-and-repeat mode. For real-time analysis, different optical labels to each nucleotide may be incorporated and multiple lasers may be utilized for stimulation of incorporated nucleotides.

Any cell type or tissue may be utilized to obtain nucleic acid samples for use in the sequencing methods described herein. In some embodiments, the DNA or RNA sample is obtained from a neoplasia/tumor or a bodily fluid, e.g., blood, obtained by known techniques (e.g. venipuncture) or saliva. Alternatively, nucleic acid tests can be performed on dry samples (e.g. hair or skin).

A variety of methods are available for detecting the presence of a particular mutation or allele in an individual's DNA or RNA. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR.

Several of the methods known in the art for detecting specific single nucleotide polymorphisms are summarized below. The method of the present invention is understood to include all available methods.

PCR based detection means may include multiplex amplification of a plurality of markers simultaneously. For example, it is well known in the art to select PCR primers to generate PCR products that do not overlap in size and can be analyzed simultaneously.

Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. In one embodiment, the single base polymorphism can be detected by using a specialized exonuclease-resistant nucleotide, as disclosed, e.g., U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (see, e.g., French Patent No. 2,650,840; PCT Application No. WO1991/02087). As in the method of U.S. Pat. No. 4,656,127, a primer may be employed that is complementary to allelic sequences immediately 3′ to a polymorphic site. The method determines the identity of the nucleotide of that site using labeled dideoxynucleotide derivatives, which, if complementary to the nucleotide of the polymorphic site, will become incorporated onto the terminus of the primer.

An alternative method, known as Genetic Bit Analysis or GBA® is described in PCT Application No. WO 1992/15712). GBA® uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO1991/02087) the GBA® method is preferably a heterogeneous phase assay, in which the primer or the target molecule is immobilized to a solid phase. Recently, several primer-guided nucleotide incorporation procedures for assaying polymorphic sites in DNA have been described (see, e.g., Komher, J. S. et al., Nucl. Acids. Res. 17:7779-7784 (1989); Sokolov, B. P., Nucl. Acids Res. 18:3671 (1990); Syvanen, A.-C, et al., Genomics 8:684-692 (1990); Kuppuswamy, M. N. et al., Proc. Natl. Acad. Sci. (U.S.A.) 88: 1143-1147 (1991); Prezant, T. R. et al., Hum. Mutat. 1: 159-164 (1992); Ugozzoli, L. et al., GATA 9: 107-112 (1992); Nyren, P. et al., Anal. Biochem. 208: 171-175 (1993)). These methods differ from GBA® in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (see, e.g., Syvanen, A.-C, et al., Amer. J. Hum. Genet. 52:46-59 (1993)).

An alternative method for identifying tumor specific neo-antigens is direct protein sequencing. Protein sequencing of enzymatic digests using multidimensional MS techniques (MSn) including tandem mass spectrometry (MS/MS)) can also be used to identify neo-antigens of the invention. Such proteomic approaches permit rapid, highly automated analysis (see, e.g., K. Gevaert and J. Vandekerckhove, Electrophoresis 21: 1145-1154 (2000)). It is further contemplated within the scope of the invention that high-throughput methods for de novo sequencing of unknown proteins may be used to analyze the proteome of a patient's tumor to identify expressed neo-antigens. For example, meta shotgun protein sequencing may be used to identify expressed neo-antigens (see, e.g., Guthals et al. (2012) Shotgun Protein Sequencing with Meta-contig Assembly, Molecular and Cellular Proteomics 11(10): 1084-96).

Tumor specific neo-antigens may also be identified using MHC multimers to identify neo-antigen-specific T-cell responses. For example, highthroughput analysis of neo-antigen-specific T-cell responses in patient samples may be performed using MHC tetramer-based screening techniques (see, e.g., Hombrink et al. (2011) High-Throughput Identification of Potential Minor Histocompatibility Antigens by MHC Tetramer-Based Screening: Feasibility and Limitations 6(8): 1-11; Hadrup et al. (2009) Parallel detection of antigen-specific T-cell responses by multidimensional encoding of MHC multimers, Nature Methods, 6(7):520-26; van Rooij et al. (2013) Tumor exome analysis reveals neoantigen-specific T-cell reactivity in an Ipilimumab-responsive melanoma, Journal of Clinical Oncology, 31: 1-4; and Heemskerk et al. (2013) The cancer antigenome, EMBO Journal, 32(2): 194-203). It is contemplated within the scope of the invention that such tetramer-based screening techniques may be used for the initial identification of tumor specific neo-antigens, or alternatively as a secondary screening protocol to assess what neo-antigens a patient may have already been exposed to, thereby facilitating the selection of candidate neo-antigens for the vaccines of the invention.

The invention further includes isolated peptides (e.g., neo-antigenic peptides containing the tumor specific mutations identified by the described methods, peptides that comprise known tumor specific mutations, and mutant polypeptides or fragments thereof identified by the described methods). These peptides and polypeptides are referred to herein as “neo-antigenic peptides” or “neo-antigenic polypeptides.” The polypeptides or peptides can be of a variety of lengths and will minimally include the small region predicted to bind to the HLA molecule of the patient (the “epitope”) as well as additional adjacent amino acids extending in both the N- and C-terminal directions. The polypeptides or peptides can be either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

In certain embodiments the size of the at least one neo-antigenic peptide molecule may comprise, but is not limited to, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the neo-antigenic peptide molecules are equal to or less than 50 amino acids. In a preferred embodiment, the neo-antigenic peptide molecules are equal to about 20 to about 30 amino acids.

As such, the present invention provides polymers (e.g., PGA) associated with (e.g., complexed, conjugated, encapsulated, absorbed, adsorbed, admixed) one or more neo-antigenic peptides. In some embodiments, the polymer is associated with (e.g., complexed, conjugated, encapsulated, absorbed, adsorbed, admixed) one neo-antigenic peptide. In some embodiments, the polymer is associated with (e.g., complexed, conjugated, encapsulated, absorbed, adsorbed, admixed) two neo-antigenic peptides. In some embodiments, the polymer is associated with (e.g., complexed, conjugated, encapsulated, absorbed, adsorbed, admixed) at least 5 or more neo-antigenic peptides. In some embodiments, polymer is associated with (e.g., complexed, conjugated, encapsulated, absorbed, adsorbed, admixed) at least about 6, about 8, about 10, about 12, about 14, about 16, about 18, or about 20 distinct peptides. In some embodiments, the polymer is associated with (e.g., complexed, conjugated, encapsulated, absorbed, adsorbed, admixed) at least 20 distinct peptides.

The neo-antigenic peptides, polypeptides, and analogs can be further modified to contain additional chemical moieties not normally part of the protein. Those derivatized moieties can improve the solubility, the biological half-life, absorption of the protein, or binding affinity. The moieties can also reduce or eliminate any desirable side effects of the proteins and the like. An overview for those moieties can be found in Remington's Pharmaceutical Sciences, 20^{ht} ed., Mack Publishing Co., Easton, Pa. (2000). For example, neo-antigenic peptides and polypeptides having the desired activity may be modified as necessary to provide certain desired attributes, e.g. improved pharmacological characteristics, while increasing or at least retaining substantially all of the biological activity of the unmodified peptide to bind the desired MHC molecule and activate the appropriate T cell. For instance, the neo-antigenic peptide and polypeptides may be subject to various changes, such as substitutions, either conservative or non-conservative, where such changes might provide for certain advantages in their use, such as improved MHC binding. Such conservative substitutions may encompass replacing an amino acid residue with another amino acid residue that is biologically and/or chemically similar, e.g., one hydrophobic residue for another, or one polar residue for another. The effect of single amino acid substitutions may also be probed using D− amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, III., Pierce), 2d Ed. (1984).

In some embodiments, the neo-antigenic peptides and polypeptides may be modified with linking agents for purposes of facilitating association with the polymers. In some embodiments, the conjugation is via a reduction sensitive linkage (e.g., pyridyldithiol propionate-thiol linkage). In some embodiments, the conjugation is via a reduction insensitive linkage (e.g., maleimide-thiol linkage).

The invention is not limited to a particular type or kind of linking agent. In some embodiments, the linking agent is a cysteine-serine-serine (CSS) molecule.

In some embodiments wherein the polymer and the neo-antigenic peptide or polypeptide is modified with CSS, the polymer is further modified with dioleoyl-sn-glycero-3-phosphoethanolamine-N-[3-(2-pyridyldithio) propionate] (DOPE-PDP) wherein upon mixing, the DOPE-PDP and CSS engage thereby resulting in a complexing (linking) of the CSS-Ag with the polymer.

The neo-antigenic peptide and polypeptides may also be modified by extending or decreasing the compound's amino acid sequence, e.g., by the addition or deletion of amino acids. The neo-antigenic peptides, polypeptides, or analogs can also be modified by altering the order or composition of certain residues. It will be appreciated by the skilled artisan that certain amino acid residues essential for biological activity, e.g., those at critical contact sites or conserved residues, may generally not be altered without an adverse effect on biological activity. The non-critical amino acids need not be limited to those naturally occurring in proteins, such as L-a-amino acids, or their D-isomers, but may include non-natural amino acids as well, such as β-γ-δ-amino acids, as well as many derivatives of L-a-amino acids.

Typically, a neo-antigen polypeptide or peptide may be optimized by using a series of peptides with single amino acid substitutions to determine the effect of electrostatic charge, hydrophobicity, etc. on MHC binding. For instance, a series of positively charged (e.g., Lys or Arg) or negatively charged (e.g., Glu) amino acid substitutions may be made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding. Amino acid substitutions are typically of single residues. Substitutions, deletions, insertions or any combination thereof may be combined to arrive at a final peptide.

One of skill in the art will appreciate that there are a variety of ways in which to produce such tumor specific neo-antigens. In general, such tumor specific neo-antigens may be produced either in vitro or in vivo. Tumor specific neo-antigens may be produced in vitro as peptides or polypeptides, which may then be formulated into a personalized neoplasia vaccine and administered to a subject. Such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide.

Alternatively, tumor specific neo-antigens may be produced in vivo by introducing molecules (e.g., DNA, RNA, viral expression systems, and the like) that encode tumor specific neo-antigens into a subject, whereupon the encoded tumor specific neo-antigens are expressed.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield R B: Solid phase peptide synthesis. I. The synthesis of a tetrapeptide. J. Am. Chem. Soc. 85:2149-54, 1963).

A further aspect of the invention provides a nucleic acid (e.g., a polynucleotide) encoding a neo-antigenic peptide of the invention, which may be used to produce the neo-antigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. A still further aspect of the invention provides an expression vector capable of expressing a polypeptide according to the invention. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

The invention further embraces variants and equivalents which are substantially homologous to the identified tumor specific neo-antigens described herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art.

The invention also includes expression vectors comprising the isolated polynucleotides, as well as host cells containing the expression vectors. It is also contemplated within the scope of the invention that the neo-antigenic peptides may be provided in the form of RNA or cDNA molecules encoding the desired neo-antigenic peptides. The invention also provides that the one or more neo-antigenic peptides of the invention may be encoded by a single expression vector. The invention also provides that the one or more neo-antigenic peptides of the invention may be encoded and expressed in vivo using a viral based system (e.g., an adenovirus system).

The term “polynucleotide encoding a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the tumor specific neo-antigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In some embodiments, the polynucleotides can comprise the coding sequence for the tumor specific neo-antigenic peptide fused in the same reading frame to a marker sequence that allows, for example, for purification of the encoded polypeptide, which may then be incorporated into the personalized neoplasia vaccine. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like. In embodiments, the polynucleotides may comprise the coding sequence for one or more of the tumor specific neo-antigenic peptides fused in the same reading frame to create a single concatamerized neo-antigenic peptide construct capable of producing multiple neo-antigenic peptides.

In embodiments, the present invention provides isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neo-antigenic peptide of the present invention.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

The isolated tumor specific neo-antigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest will be inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host. Recombinant expression vectors may be used to amplify and express DNA encoding the tumor specific neo-antigenic peptides. Recombinant expression vectors are replicable DNA constructs which have synthetic or cDNA-derived DNA fragments encoding a tumor specific neo-antigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transforaiants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23: 175, 1981), and other cell lines capable of expressing an appropriate vector including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein. Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

As such, in certain embodiments, the present invention relates to personalized strategies for the treatment of disorders (e.g., neoplasia), and more particularly tumors, by administering a therapeutically effective amount of a polymer moiety comprising a polymer associated with associated with (e.g., complexed, conjugated, encapsulated, absorbed, adsorbed, admixed) one or more neoplasia/tumor specific neo-antigens to a subject (e.g., a mammal such as a human) (e.g., a vaccine composition capable of raising a specific T-cell response). Indeed, in certain embodiments, whole genome/exome sequencing may be used to identify all, or nearly all, mutated neo-antigens that are uniquely present in a neoplasia/tumor of an individual patient, and that this collection of mutated neo-antigens may be analyzed to identify a specific, optimized subset of neo-antigens for use as a personalized cancer vaccine for treatment of the patient's neoplasia/tumor. For example, in some embodiments, a population of neoplasia/tumor specific neo-antigens may be identified by sequencing the neoplasia/tumor and normal DNA of each patient to identify tumor-specific mutations, and determining the patient's HLA allotype. The population of neoplasia/tumor specific neo-antigens and their cognate native antigens may then be subject to bioinformatic analysis using validated algorithms to predict which tumor-specific mutations create epitopes that could bind to the patient's HLA allotype, and in particular which tumor-specific mutations create epitopes that could bind to the patient's HLA allotype more effectively than the cognate native antigen. Based on this analysis, one or more peptides corresponding to a subset of these mutations may be designed and synthesized for each patient, and pooled together for use as a cancer vaccine in immunizing the patient. The neo-antigens peptides may be combined another anti-neoplastic agent. In some embodiments, such neo-antigens are expected to bypass central thymic tolerance (thus allowing stronger antitumor T cell response), while reducing the potential for autoimmunity (e.g., by avoiding targeting of normal self-antigens).

The invention further provides a method of inducing a neoplasia/tumor specific immune response in a subject, vaccinating against a neoplasia/tumor, treating and or alleviating a symptom of cancer in a subject by administering the subject a neo-antigenic peptide or vaccine composition of the invention.

According to the invention, the above-described cancer vaccine may be used for a patient that has been diagnosed as having cancer, or at risk of developing cancer. In one embodiment, the patient may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

The peptide or composition of the invention is administered in an amount sufficient to induce a CTL response. The neo-antigenic peptide, polypeptide or vaccine composition of the invention can be administered alone or in combination with other therapeutic agents. The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. For prostate cancer treatment, a preferred chemotherapeutic agent with which anti-CTLA-4 can be combined is paclitaxel (Taxol®).

In addition, the subject may be further administered an anti-immunosuppressive or immuno stimulatory agent. For example, the subject is further administered an anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, and inhibitors of IDO. Blockade of CTLA-4 or PD-1/PD-L1 by antibodies can enhance the immune response to cancerous cells in the patient. In particular, CTLA-4 blockade has been shown effective when following a vaccination protocol.

The optimum amount of each peptide to be included in the vaccine composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c, i.d., i.p., i.m., and i.v. Preferred methods of DNA or RNA (e.g., siRNA, mRNA) injection include i.d., i.m., s.c, i.p. and i.v. For example, doses of between 1 and 500 mg 50 μg and 1.5 mg, preferably 10 μg to 500 μg, of peptide or DNA or RNA (e.g., siRNA, mRNA) may be given and will depend from the respective peptide or DNA or RNA (e.g., siRNA, mRNA). Doses of this range were successfully used in previous trials (Brunsvig P F, et al., Cancer Immunol Immunother. 2006; 55(12): 1553-1564; M. Staehler, et al., ASCO meeting 2007; Abstract No 3017). Other methods of administration of the vaccine composition are known to those skilled in the art.

The inventive vaccine may be compiled so that the selection, number and/or amount of peptides present in the composition is/are tissue, cancer, and/or patient-specific. For instance, the exact selection of peptides can be guided by expression patterns of the parent proteins in a given tissue to avoid side effects. The selection may be dependent on the specific type of cancer, the status of the disease, earlier treatment regimens, the immune status of the patient, and, of course, the HLA-haplotype of the patient. Furthermore, the vaccine according to the invention can contain individualized components, according to personal needs of the particular patient. Examples include varying the amounts of peptides according to the expression of the related neoantigen in the particular patient, unwanted side-effects due to personal allergies or other treatments, and adjustments for secondary treatments following a first round or scheme of treatment.

Such vaccines may be administered to an individual already suffering from cancer. In therapeutic applications, such vaccines are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition and possibly by measuring specific CTL activity in the patient's blood. It should be kept in mind that the peptide and compositions of the present invention may generally be employed in serious disease states, that is, life-threatening or potentially life threatening situations, especially when the cancer has metastasized. For therapeutic use, administration should begin as soon as possible after the detection or surgical removal of tumors. This is followed by boosting doses until at least symptoms are substantially abated and for a period thereafter. The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor.

Such embodiments are not limited to a particular type of adjuvant. Generally, adjuvants are any substance whose admixture into the vaccine composition increases or otherwise modifies the immune response to the mutant peptide. Carriers are scaffold structures, for example a polypeptide or a polysaccharide, to which the antigenic peptide (e.g., neo-antigenic peptide) is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Suitable adjuvants include, but are not limited to 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juylmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel® vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418). Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS).

Recognition of these “danger signals” activates multiple elements of the innate and adaptive immune system. TLRs are expressed by cells of the innate and adaptive immune systems such as dendritic cells (DCs), macrophages, T and B cells, mast cells, and granulocytes and are localized in different cellular compartments, such as the plasma membrane, lysosomes, endosomes, and endolysosomes. Different TLRs recognize distinct PAMPS. For example, TLR4 is activated by LPS contained in bacterial cell walls, TLR9 is activated by unmethylated bacterial or viral CpG DNA, and TLR3 is activated by double stranded RNA. TLR ligand binding leads to the activation of one or more intracellular signaling pathways, ultimately resulting in the production of many key molecules associated with inflammation and immunity (particularly the transcription factor NF-κB and the Type-I interferons). TLR mediated DC activation leads to enhanced DC activation, phagocytosis, upregulation of activation and co-stimulation markers such as CD80, CD83, and CD86, expression of CCR7 allowing migration of DC to draining lymph nodes and facilitating antigen presentation to T cells, as well as increased secretion of cytokines such as type I interferons, IL-12, and IL-6. All of these downstream events are critical for the induction of an adaptive immune response.

Other receptors which may be targeted include the toll-like receptors (TLRs). TLRs recognize and bind to pathogen-associated molecular patterns (PAMPs). PAMPs target the TLR on the surface of the dendritic cell and signals internally, thereby potentially increasing DC antigen uptake, maturation and T-cell stimulatory capacity. PAMPs conjugated to the particle surface or co-encapsulated include unmethylated CpG DNA (bacterial), double-stranded RNA (viral), lipopolysacharride (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

Among the most promising cancer vaccine adjuvants currently in clinical development are the TLR9 agonist CpG and the synthetic double-stranded RNA (dsRNA) TLR3 ligand poly-ICLC. In preclinical studies poly-ICLC appears to be the most potent TLR adjuvant when compared to LPS and CpG due to its induction of pro-inflammatory cytokines and lack of stimulation of IL-10, as well as maintenance of high levels of co-stimulatory molecules in DCs. Furthermore, poly-ICLC was recently directly compared to CpG in non-human primates (rhesus macaques) as adjuvant for a protein vaccine consisting of human papillomavirus (HPV)16 capsomers (Stahl-Hennig C, Eisenblatter M, Jasny E, et al. Synthetic double-stranded RNAs are adjuvants for the induction of T helper 1 and humoral immune responses to human papillomavirus in rhesus macaques. PLoS pathogens. April 2009; 5(4)).

In some embodiments, the adjuvant is a dendritic cell targeting molecule (DC). DC is potent and is responsible for initiating antigen-specific immune responses. One biological feature of DCs is their ability to sense conditions under which antigen is encountered, initiating a process of “DC maturation”. Using receptors for various microbial and inflammatory products, DCs respond to antigen exposure in different ways depending on the nature of the pathogen (virus, bacteria, protozoan) encountered. This information is transmitted to T cells by altered patterns of cytokine release at the time of antigen presentation in lymph nodes, altering the type of T cell response elicited. Thus, targeting DCs provides the opportunity not only to quantitatively enhance the delivery of antigen and antigen responses in general, but to qualitatively control the nature of the immune response depending on the desired vaccination outcome.

Dendritic cells express a number of cell surface receptors that can mediate the endocytosis of bound antigen. Targeting exogenous antigens to internalizing surface molecules on systemically-distributed antigen presenting cells facilitates uptake of antigens and thus overcomes a major rate-limiting step in immunization and thus in vaccination.

Dendritic cell targeting molecules include monoclonal or polyclonal antibodies or fragments thereof that recognize and bind to epitopes displayed on the surface of dendritic cells. Dendritic cell targeting molecules also include ligands which bind to a cell surface receptor on dendritic cells. One such receptor, the lectin DEC-205, has been used in vitro and in mice to boost both humoral (antibody-based) and cellular (CD8 T cell) responses by 2-4 orders of magnitude (see, e.g., Hawiger, et al., J. Exp. Med., 194(6):769-79 (2001); Bonifaz, et al., J. Exp. Med., 196(12):1627-38 (2002); Bonifaz, et al., J. Exp. Med., 199(6):815-24 (2004)).

A variety of other endocytic receptors, including a mannose-specific lectin (mannose receptor) and IgG Fc receptors, have also been targeted in this way with similar enhancement of antigen presentation efficiency. Other suitable receptors which may be targeted include, but are not limited to, DC-SIGN, 33D1, SIGLEC-H, DCIR, CD11c, heat shock protein receptors and scavenger receptors.

In some embodiments, the adjuvant is CpG. CpG immuno stimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Th1 cells and strong cytotoxic T-lymphocyte (CTL) generation, even in the absence of CD4 T-cell help. The Th1 bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, Jun. 2006, 471-484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a preferred component of the pharmaceutical composition of the present invention. Other TLR binding molecules such as RNA binding TLR 7, TLR 8 and/or TLR 9 may also be used.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5, 6-Dimethylxanthenone-4-Acetic Acid, Journal of Immunology, 190:5216-25 and Kim et al. (2013) Anticancer Flavonoids are Mouse-Selective STING Agonists, 8: 1396-1401). Other examples of useful adjuvants include, but are not limited to, chemically modified CpGs (e.g. CpR, Idera), Poly(I:C) e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyl and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′ 5′-OAS and the P1/eIF2a kinase, also known as the PKR (4-6), as well as RIG-I helicase and MDA5.

Such methods are not limited to generating polymer moieties having a polymer associated with an antigen and an adjuvant (e.g., dendritic cell targeting molecule). In some embodiments, the antigen and adjust are conjugated to outer surface of the polymer.

In some embodiments, the polymer moiety is synthesized with thiol-reactive phospholipids that permit reduction-sensitive linkage of the antigen and/or adjuvant with the polymer. In some embodiments, loading of the DC within the polymer moiety is facilitated through cholesterol modification of the DC molecule. In some embodiments, lyophilization methods are used for the preparation of homogenous polymer moiety. In some embodiments, phospholipids and ApoA mimetic peptides are dissolved in glacial acetic acid and lyophilized. In some embodiments, antigen peptides are incubated with the polymer in a buffer (e.g., a sodium phosphate buffer (pH 7.4)) (e.g., at room temperature for 3 hours) to allow for the conjugation of antigen peptides. In some embodiments, the unconjugated antigen peptides are removed using a desalting column (MWCO=7000 Da). In some embodiments, incorporation of the cholesterol modified DC (Cho-DC) to the polymer involves incubation with the polymer at room temperature for approximately 30 min.

Such embodiments are not limited to a particular manner of characterizing the polymer moiety having a polymer conjugated with antigen and DC. In some embodiments, the morphology of the polymer moeity is observed by TEM. In some embodiments, the size distribution of the polymer moeity is analyzed by dynamic light scattering (DLS) using a Malven Nanosizer instrument and GPC assay.

The polymer moieties described herein are configured to activate an immune response (e.g., polymer-αGalCer) (e.g., Ag/DC-polymer) are useful for activating T cells in subjects for prophylactic and therapeutic applications. Activation of T cells by polymer vaccine compositions increases their proliferation, cytokine production, differentiation, effector functions and/or survival. Methods for measuring these are well known to those in the art. The T cells activated by the polymer vaccine compositions can be any cell which express the T cell receptor, including α/β and γ/δ T cell receptors. T-cells include all cells which express CD3, including T-cell subsets which also express CD4 and CD8. T-cells include both naïve and memory cells and effector cells such as CTL. T-cells also include regulatory cells such as Th1, Tc1, Th2, Tc2, Th3, Treg, and Tr1 cells. T-cells also include NKT-cells and similar unique classes of the T-cell lineage. In some embodiments, the T cells that are activated are CD8⁺ T cells.

In general, compositions comprising the polymer moieties configured to activate an immune response (e.g., polymer-αGalCer) (e.g., Ag/DC-polymer) are useful for treating a subject having or being predisposed to any disease or disorder to which the subject's immune system mounts an immune response. The compositions are useful as prophylactic vaccines, which confer resistance in a subject to subsequent exposure to infectious agents. The compositions are also useful as therapeutic vaccines, which can be used to initiate or enhance a subject's immune response to a pre-existing antigen, such as a tumor antigen in a subject with cancer, or a viral antigen in a subject infected with a virus. The compositions are also useful as desensitizing vaccines, which function to “tolerize” an individual to an environmental antigen, such as an allergen.

The ability to target these compositions to professional antigen-presenting cells such as dendritic cells, and the ability of these compositions to elicit T-cell mediated immune responses by causing cross-presentation of antigens makes these compositions especially useful for eliciting a cell-mediated response to a disease-related antigen in order to attack the disease. Thus, in some embodiments, the type of disease to be treated or prevented is a malignant tumor or a chronic infectious disease caused by a bacterium, virus, protozoan, helminth, or other microbial pathogen that enters intracellularly and is attacked, i.e., by the cytotoxic T lymphocytes.

The desired outcome of a prophylactic, therapeutic or de-sensitized immune response may vary according to the disease, according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease. For example, the stimulation of an immune response against a cancer may be coupled with surgical, chemotherapeutic, radiologic, hormonal and other immunologic approaches in order to affect treatment.

Subjects with or exposed to infectious agents can be treated therapeutically or prophylactically the polymer moieties configured to activate an immune response (e.g., polymer-αGalCer) (e.g., Ag/DC-polymer) as disclosed herein. Infectious agents include bacteria, viruses and parasites. In some instances, the subject can be treated prophylactically, such as when there may be a risk of developing disease from an infectious agent. An individual traveling to or living in an area of endemic infectious disease may be considered to be at risk and a candidate for prophylactic vaccination against the particular infectious agent. Preventative treatment can be applied to any number of diseases where there is a known relationship between the particular disease and a particular risk factor, such as geographical location or work environment.

Subjects with or at risk for developing malignant tumors can be treated therapeutically or prophylactically the polymer moieties configured to activate an immune response (e.g., polymer-αGalCer) (e.g., Ag/DC-polymer) as disclosed herein. In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site. The polymer moieties configured to activate an immune response (e.g., polymer-αGalCer) (e.g., Ag/DC-polymer) as disclosed herein are useful for treating subjects having malignant tumors.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. A melanoma is a type of carcinoma of the skin for which this invention is particularly useful. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated in with the provided polymer moieties configured to activate an immune response (e.g., polymer-αGalCer) (e.g., Ag/DC-polymer) include, but are not limited to, the following: bladder, brain, breast, cervical, colo-rectal, esophageal, kidney, liver, lung, nasopharyngeal, pancreatic, prostate, skin, stomach, uterine, and the like. Administration is not limited to the treatment of an existing tumor or infectious disease but can also be used to prevent or lower the risk of developing such diseases in an individual, i.e., for prophylactic use. Potential candidates for prophylactic vaccination include individuals with a high risk of developing cancer, i.e., with a personal or familial history of certain types of cancer.

Subjects with or at risk for exposure to allergens can be treated therapeutically or prophylactically the polymer moieties configured to activate an immune response (e.g., polymer-αGalCer) (e.g., Ag/DC-polymer) as disclosed herein. Such polymer moieties may be administered to subjects for the purpose of preventing and/or attenuating allergic reactions, such as allergic reactions which lead to anaphylaxis. Allergic reactions may be characterized by the T_{H}2 responses against an antigen leading to the presence of IgE antibodies. Stimulation of T_{H}1 immune responses and the production of IgG antibodies may alleviate allergic disease. Thus, the polymer moieties configured to activate an immune response (e.g., polymer-αGalCer) (e.g., Ag/DC-polymer) as disclosed herein are useful for producing antibodies that prevent and/or attenuate allergic reactions in subjects exposed to allergens.

Subjects with or at risk for immunosuppressed conditions can be treated therapeutically or prophylactically the polymer moieties configured to activate an immune response (e.g., polymer-αGalCer) (e.g., Ag/DC-polymer) as disclosed herein. The polymer moiety based vaccines disclosed herein can be used for treatment of disease conditions characterized by immunosuppression, including, but not limited to, AIDS or AIDS-related complex, idiopathic immuno suppression, drug induced immunosuppression, other virally or environmentally-induced conditions, and certain congenital immune deficiencies. Such polymer moiety based vaccine compositions can also be employed to increase immune function that has been impaired by the use of radiotherapy of immunosuppressive drugs (e.g., certain chemotherapeutic agents), and therefore can be particularly useful when used in conjunction with such drugs or radiotherapy.

Subjects with or at risk for coronary heart disease and/or elevated LDL-C levels can be treated therapeutically or prophylactically the polymer moieties configured to activate an immune response as disclosed herein. While effectiveness of mAb therapy against PCSK9 has established (see, e.g., Banerjee, Y.; et al., New England Journal of Medicine 2012, 366 (25), 2425-2426; Stein, E. A.; et al., Circulation 2013, 128 (19), 2113-2120), development of more durable PCSK9 vaccines are needed. In addition, one of the challenges for PCSK9 vaccines is that self antigens, such as PCSK9 peptides, are not immunogenic, unless they are coupled to vaccine/adjuvant systems that can efficiently co-deliver antigens and immunostimulatory molecules to immune cells (see, e.g., Krishnamachari, Y.; et al., Advanced Drug Delivery Reviews 2009, 61 (3), 205-217; Hamdy, S.; et al., Advanced Drug Delivery Reviews 2011, 63 (10-11), 943-955).

Embodiments of the present invention wherein polymer moieties are conjugated with a PCSK9-antigen and a CpG-adjuvant (PCSK9-Ag/CpG-polymer) address such needs. Indeed, vaccination against PCSK9 with PCSK9-Ag/CpG-polymer embodiments effectively inhibits interaction between PCSK9 and LDLR, while avoiding the need for repeated injections of expensive mAb (see, e.g., Fattori, E.; et al., Journal of Lipid Research 2012, 53 (8), 1654-1661; Gergana Galabova, et al., PLOS ONE 2014, 9 (12)).

In general, methods of administering vaccines as disclosed herein (e.g., polymer moieties configured to activate an immune response (e.g., polymer-αGalCer) (e.g., Ag/DC-polymer)) are well known in the art. Any acceptable method known to one of ordinary skill in the art may be used to administer a formulation to the subject. The administration may be localized (i.e., to a particular region, physiological system, tissue, organ, or cell type) or systemic. Vaccines can be administered by a number of routes including, but not limited to: oral, inhalation (nasal or pulmonary), intravenous, intraperitoneal, intramuscular, transdermal, subcutaneous, topical, sublingual, or rectal means. Injections can be e.g., intravenous, intradermal, subcutaneous, intramuscular, or intraperitoneal. In some embodiments, the injections can be given at multiple locations.

Administration of the formulations may be accomplished by any acceptable method which allows an effective amount of the vaccine to reach its target. The particular mode selected will depend upon factors such as the particular formulation, the severity of the state of the subject being treated, and the dosage required to induce an effective immune response. As generally used herein, an “effective amount” is that amount which is able to induce an immune response in the treated subject. The actual effective amounts of vaccine can vary according to the specific antigen or combination thereof being utilized, the particular composition formulated, the mode of administration, and the age, weight, condition of the individual being vaccinated, as well as the route of administration and the disease or disorder.

In certain embodiments, glycolipids encapsulated within polymer moieties are used as stimulators of natural killer T cell-mediated immune responses.

Natural killer T (NKT) cells are a heterogeneous group of T cells that share properties of both T cells and natural killer cells. Many of these cells recognize the non-polymorphic CD1 d molecule, an antigen-presenting molecule that binds self and foreign lipids and glycolipids. NKT cells constitute only approximately 0.1% of all peripheral blood T cells. NKT cells are a subset of T cells that coexpress an αβ T-cell receptor, but also express a variety of molecular markers that are typically associated with NK cells, such as NK1.1. The best-known NKT cells differ from conventional al T cells in that their T-cell receptors are far more limited in diversity (‘invariant’ or ‘type 1’ NKT). They and other CD d-restricted T cells (‘type 2’ NKT) recognize lipids and glycolipids presented by CD1d molecules, a member of the CD1 family of antigen-presenting molecules, rather than peptide-major histocompatibility complexes (MHCs). NKT cells include both NK1.1⁺ and NK1.1⁻, as well as CD4⁺, CD4⁻, CD8⁺ and CD8⁻ cells.

In some embodiments the glycolipid is the synthetic glycolipid alpha-galactosylceramide (αGalCer). Dendritic cells presenting antigens in the context of CD1d can lead to rapid innate and prolonged production of cytokines such as interferon and IL-4 by natural killer T cells (NKT cells). CD1d is a major histocompatibility complex class I-like molecule that presents glycolipid antigens to a subset of NKT cells. Advantageously, αGalCer is not toxic to humans and has been shown to act as an adjuvant, priming both antigen-specific CD4+ and CD8+ T cell responses. For example, it has been shown that αGalCer in conjunction with a malaria vaccine can lead to cytotoxic responses against infected cells, which is an ideal scenario for vaccines against infectious diseases. In addition to αGalCer, other glycolipids that function as adjuvants to activate NKT cell-mediated immune responses can be used.

Such embodiments are not limited to a particular manner of assessing the delivery profile of the αGalCer in vitro and in vivo. In some embodiments, labelling the molecules with an imaging agent (e.g., fluorescent dye Cy3) permits visualization of the biodistribution of αGalCer molecules at the organ level and also the intracellular delivery profile.

In certain embodiments, the present invention provides methods for inducing a natural killer T cell-mediated immune response in a cell comprising exposing the cell to a composition comprising an αGalCer glycolipid encapsulated within a polymer moiety, wherein such exposure results in the induction of a natural killer T cell-mediated immune response. In some embodiments, the cells are in vivo cells. In some embodiments, the cells are in vitro cells. In some embodiments, the cells are ex vivo cells.

In certain embodiments, the present invention provides methods for inducing a natural killer T cell-mediated immune response in a subject (e.g., a human patient) comprising administering to the patient a pharmaceutical composition comprising an αGalCer glycolipid conjugated with a polymer (e.g., PGA) (e.g., conjugated via a reduction sensitive linker) (e.g., conjugated via a reduction insensitive linker), wherein such administration results in the induction of a natural killer T cell-mediated immune response.

### ADDITIONAL EMBODIMENTS

In certain embodiments, the polymer moieties as described herein (e.g., configured for RNA Interference) (e.g., configured for activating an immune response) are associated with (e.g., complexed, conjugated, encapsulated, absorbed, adsorbed, admixed) one or more therapeutic agents. Such embodiments are not limited to particular type or kind of therapeutic agent.

In some embodiments, the therapeutic agent configured for treating and/or preventing cancer. Examples of such therapeutic agents include, but are not limited to, chemotherapeutic agents, anti-oncogenic agents, anti-angiogenic agents, tumor suppressor agents, anti-microbial agents, etc.

In some embodiments, the therapeutic agent is configured for treating and/or preventing autoimmune disorders and/or inflammatory disorders. Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, parenteral gold or oral gold.

In some embodiments, the therapeutic agent is configured for treating and/or preventing cardiovascular related disorders (e.g., atherosclerosis, heart failure, arrhythmia, atrial fibrillation, hypertension, coronary artery disease, angina pectoris, etc.). Examples of therapeutic agents known to be useful in treating and/or preventing cardiovascular related disorders include, angiotensin-converting enzyme (ACE) inhibitors (e.g., benazepril, enalapril, Lisinopril, perindopril, Ramipril), adenosine, alpha blockers (alpha adrenergic antagonist medications) (e.g., clonidine, guanabenz, labetalol, phenoxybenzamine, terazosin, doxazosin, guanfacine, methyldopa, prazosin), angtiotensin II receptor blockers (ARBs) (e.g., candesartan, irbesartan, olmesartan medoxomil, telmisartan, eprosartan, losartan, tasosartan, valsartan), antiocoagulants (e.g., heparin fondaparinux, warfarin, ardeparin, enoxaparin, reviparin, dalteparin, nadroparin, tinzaparin), antiplatelet agents (e.g., abciximab, clopidogrel, eptifibatide, ticlopidine, cilostazol, dipyridamole, sulfinpyrazone, tirofiban), beta blockers (e.g., acebutolol, betaxolol, carteolol, metoprolol, penbutolol, propranolol, atenolol, bisoprolol, esmolol, nadolol, pindolol, timolol), calcium channel blockers (e.g., amlopidine, felodipine, isradipine, nifedipine, verapamil, diltiazem, nicardipine, nimodipine, nisoldipine), diuretics, aldosterone blockers, loop diuretics (e.g., bumetanide, furosemide, ethacrynic acid, torsemide), potassium-sparing diuretics, thiazide diuretics (e.g., chlorothiazide, chlorthalidone, hydrochlorothiazide, hydroflumethiazide, methyclothiazide, metolazone, polythiazide, quinethazone, trichlormethiazide), inoptropics, bile acid sequestrants (e.g., cholestyramine, coletipol, colesevelam), fibrates (e.g., clofibrate, gemfibrozil, fenofibrate), statins (e.g., atorvastatinm, lovastatin, simvastatin, fluvastatin, pravastatin), selective cholesterol absorption inhibitors (e.g., ezetimibe), potassium channel blockers (e.g., amidarone, ibutilide, dofetilide), sodium channel blockers (e.g., disopyramide, mexiletine, procainamide, quinidine, flecainide, moricizine, propafenone), thrombolytic agents (e.g., alteplase, reteplase, tenecteplase, anistreplase, streptokinase, urokinase), vasoconstrictors, vasodilators (e.g., hydralazine, minoxidil, mecamylamine, isorbide dintrate, isorbide mononitrate, nitroglycerin).

In some embodiments, the polymer moieties are further associated with (e.g., complexed, conjugated, encapsulated, absorbed, adsorbed, admixed) agents useful for determining the location of administered particles. Agents useful for this purpose include fluorescent tags, radionuclides and contrast agents.

Suitable imaging agents include, but are not limited to, fluorescent molecules such as those described by Molecular Probes (Handbook of fluorescent probes and research products), such as Rhodamine, fluorescein, Texas red, Acridine Orange, Alexa Fluor (various), Allophycocyanin, 7-aminoactinomycin D, BOBO-1, BODIPY (various), Calcien, Calcium Crimson, Calcium green, Calcium Orange, 6-carboxyrhodamine 6G, Cascade blue, Cascade yellow, DAPI, DiA, DID, Di1, DiO, DiR, ELF 97, Eosin, ER Tracker Blue-White, EthD-1, Ethidium bromide, Fluo-3, Fluo4, FM1-43, FM4-64, Fura-2, Fura Red, Hoechst 33258, Hoechst 33342, 7-hydroxy-4-methylcoumarin, Indo-1, JC-1, JC-9, JOE dye, Lissamine rhodamine B, Lucifer Yellow CH, LysoSensor Blue DND-167, LysoSensor Green, LysoSensor Yellow/Blu, Lysotracker Green FM, Magnesium Green, Marina Blue, Mitotracker Green FM, Mitotracker Orange CMTMRos, MitoTracker Red CMXRos, Monobromobimane, NBD amines, NeruoTrace 500/525 green, Nile red, Oregon Green, Pacific Blue. POP-1, Propidium iodide, Rhodamine 110, Rhodamine Red, R-Phycoerythrin, Resorfin, RH414, Rhod-2, Rhodamine Green, Rhodamine 123, ROX dye, Sodium Green, SYTO blue (various), SYTO green (Various), SYTO orange (various), SYTOX blue, SYTOX green, SYTOX orange, Tetramethylrhodamine B, TOT-1, TOT-3, X-rhod-1, YOYO-1, YOYO-3. In some embodiments, ceramides are provided as imaging agents. In some embodiments, SiP agonists are provided as imaging agents.

Additionally radionuclides can be used as imaging agents. Suitable radionuclides include, but are not limited to radioactive species of Fe(III), Fe(II), Cu(II), Mg (II), Ca (II), and Zn(II) Indium, Gallium and Technetium. Other suitable contrast agents include metal ions generally used for chelation in paramagnetic T1-type MIR contrast agents, and include di- and tri-valent cations such as copper, chromium, iron, gadolinium, manganese, erbium, europium, dysprosium and holmium. Metal ions that can be chelated and used for radionuclide imaging, include, but are not limited to metals such as gallium, germanium, cobalt, calcium, indium, iridium, rubidium, yttrium, ruthenium, yttrium, technetium, rhenium, platinum, thallium and samarium. Additionally metal ions known to be useful in neutron-capture radiation therapy include boron and other metals with large nuclear cross-sections. Also suitable are metal ions useful in ultrasound contrast, and X-ray contrast compositions.

Examples of other suitable contrast agents include gases or gas emitting compounds, which are radioopaque.

In some embodiments, the polymer moieties are further associated with (e.g., complexed, conjugated, encapsulated, absorbed, adsorbed, admixed) a targeting agent (TA). In some embodiments, targeting agents are used to assist in delivery of the polymer-TA to desired body regions (e.g., bodily regions affected by a cardiovascular related disorder). Examples of targeting agents include, but are not limited to, an antibody, receptor ligand, hormone, vitamin, and antigen, however, the present invention is not limited by the nature of the targeting agent. In some embodiments, the antibody is specific for a disease-specific antigen. In some embodiments, the receptor ligand includes, but is not limited to, a ligand for CFTR, EGFR, estrogen receptor, FGR2, folate receptor, IL-2 receptor, glycoprotein, and VEGFR. In some embodiments, the receptor ligand is folic acid.

In some embodiments, the polymer moieties of the present invention may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (SCIMED LIFE SYSTEMS, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

In some embodiments, the present invention also provides kits comprising polymer moieties as described herein. In some embodiments, the kits comprise one or more of the reagents and tools necessary to generate polymer moieties, and methods of using such polymer moieties.

The polymer moieties of the present invention may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the polymer moieties population and are important in the production quality control for eventual use in in vivo applications.

Where clinical applications are contemplated, in some embodiments of the present invention, polymer moieties are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight polymer moiety may be administered using one or more of the routes described herein.

In preferred embodiments, the polymer moieties are used in conjunction with appropriate salts and buffers to render delivery of the compositions in a stable manner to allow for uptake by target cells. Buffers also are employed when the polymer moieties are introduced into a patient. Aqueous compositions comprise an effective amount of the polymer moieties to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active polymer moieties may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active polymer moieties in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, polymer moieties are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. The polymer moieties also may be formulated as inhalants.

The present invention also includes methods involving co-administration of the polymer moieties as described herein with one or more additional active agents. Indeed, it is a further aspect of this invention to provide methods for enhancing prior art therapies and/or pharmaceutical compositions by co-administering the polymer moieties of this invention. In co-administration procedures, the agents may be administered concurrently or sequentially. In some embodiments, the polymer moieties described herein are administered prior to the other active agent(s). The agent or agents to be co-administered depends on the type of condition being treated.

The present disclosure further provides kits comprising compositions comprising polymer moieties as described herein or the ingredients necessary to synthesize the polymer moieties as described herein. In some embodiments, the kit includes all of the components necessary, sufficient or useful for administering such polymer moieties.

### EXPERIMENTAL

The following examples are provided in order to demonstrate and further illustrate certain preferred embodiments and aspects of the present invention and are not to be construed as limiting the scope thereof.

### Example I

This example describes peptide antigens and tumor neo-antigens conjugated on poly(L-glutamic acid) polypeptides for vaccine delivery applications.

### Materials

Poly-L-glutamic acid sodium salt (PGA, Mw 30 kD, 120 kD) was purchased from Alamanda Polymers (Huntsville, Ala.). N-(3-Dimethylaminopropyl)-N′-ethylcarbodiimide hydrochloride (EDC) and Dithiothreitol (DTT) were from Thermo Scientific (Waltham, Mass.). N-hydroxysuccinimide (NHS) and 4′,6-Diamidine-2′-phenylindole dihydrochloride (DAPI) were from Sigma-Aldrich (St. Louis, Mo.). N-(2-Aminoethyl)maleimide hydrochloride (MAL-NH₂) and (S)-2-Pyridylthio cysteamine hydrochloride (PDP-NH₂) were from TCI America (Portland, Oreg.) and Combi-Blocks (San Diego, Calif.), respectively. Antigen peptides including SIINFEKL, CSSSIINFEKL, CSSSIINFEKL-FITC, and ASMTNMELM, and murine GM-CSF were supplied by GenScript (Piscataway, N.J.). Peptide CSSASMTNMELM was synthesized by AnaSpec (Fremont, Calif.). The Toll-like receptor 9 agonist CpG oligonucleotides was from InvivoGen (San Diego, Calif.). Antibodies including anti-CD16/32, anti-CD40-APC, anti-CD86-PE, and anti-SIINFEKL-H-2K^{b}-PE were from eBioscience (San Diego, Calif.). Anti-CD11c-PECy7 and anti-CD8-APC were from BD Biosciences (San Jose, Calif.). PE-labeled tetramers including SIINFEKL-H-2Kb and ASMTNMELM-H-2D^{b} were kindly provided by NIH tetramer core facility. Carboxyfluorescein succinimidyl ester (CFSE), phosphate buffered saline (PBS), RPMI 1640 media, fetal bovine serum (FBS), penicillin-streptomycin, β-mercaptoethanol, and ACK lysis buffer were all from Life Technologies (Grand Island, N.Y.).

### Synthesis of PGA-Peptide and PGA-Neo-Antigen Conjugates

PGA, EDC, NHS, and MAL-NH₂ or PDP-NH₂ (carboxyl groups:EDC:NHS:linker=1:0.1:0.1:0.01 or 1:0.5:0.5:0.05, m/m/m/m) were dissolved in PBS (pH 7.4) and reacted for 5 h at room temperature, followed by purification using a Zeba desalting column (7 kD MWCO) and lyophilization. Amount of PDP modified in PGA was quantified by adding 10 μl of 0.1 M DTT solution to 100 μl of the conjugate solution (1 mg/ml in PBS), reacting for 15 min at room temperature, and measuring absorbance at 343 nm using a micro-plate reader (BioTek); while MAL content was quantified by measuring absorbance of the conjugate solution at 300 nm. For synthesis of disulfide bond-linked PGA-peptide conjugates (PGA-SS-peptide sequence), CSSSIINFEKL, CSSSIINFEKL-FITC, or CSSASMTNMELM were added to the aqueous solution of PGA conjugates (peptide:linker=3-6:1, m/m), and reacted for 2 h at room temperature, followed by purification and lyophilization as above. Reaction was monitored by measuring absorbance of the reaction mixture at 343 nm. Similar conditions were used to synthesize non-sensitive bond-linked PGA-peptide conjugates (PGA-MAL-peptide sequence).

### Gel Permeation Chromatography (GPC)

PGA and PGA conjugates were dissolved in PBS and eluted with PBS at 1 ml/min, room temperature, using a GPC column (Agilent PL aquagel-OH MIXED-M, 7.5×300 mm, 8 μm). Absorbance at 220 nm was measured by a high performance liquid chromatography (HPLC) system (SHIMADZU).

### Preparation of Murine Bone Marrow-Derived Dendritic Cells (BMDCs)

Femur and tibia were harvested from C57BL/6 mice. Cells were collected by flushing bone marrow with a syringe, passing the cell suspension through a cell strainer (mesh size=40 μm), followed by centrifugation (200×g, 5 min). Cells were then seeded into non-tissue culture treated petri-dishes at a density of 2×10⁶ cells/ml in DC culture media (RPMI 1640 supplemented with 10% FBS, 1% penicillin-streptomycin, 50 μM β-mercaptoethanol, and 20 ng/ml GM-CSF), cultured at 37° C. with 5% CO₂. Culture media were refreshed on days 3, 6, and 8, and BMDCs were used on days 8-12.

### BMDC Activation and Cross-Presentation of Antigens

BMDCs were seeded at a density of 8×10⁵ cells/ml into 12-well plates. Cells were incubated with PGA₁₂₀ₖ-MAL-CSSSIINFEKL or PGA₁₂₀ₖ-SS-CSSSIINFEKL (1% modification of carboxyl groups, 2 nmol SIINFEKL/ml) w/or w/o 0.5 μg/ml CpG in 1 ml BMDC culture media for 24 h. Cells were harvested, blocked with anti-CD16/32 at room temperature for 10 min, and then stained with anti-CD11c-PECy7, anti-CD40-APC, anti-CD86-PE, or anti-SIINFEKL-H-2K^{b}-PE at room temperature for 30 min. Finally, cells were washed and resuspended in 2 μg/ml DAPI solution and analyzed by a flow cytometer (Cyan 5, Beckman Coulter).

### Proliferation of Antigen-Specific CD8⁺ T Cells In Vitro

SIINFEKL-specific CD8⁺ T cells were isolated from OT-I mice using an EasySep™ Mouse CD8⁺ T Cell Isolation Kit (STEMCELL Technologies, Cambridge, Mass.), and stained by CFSE. 5×10⁴ BMDCs were seeded into 96-well plates, and incubated with PGA₁₂₀ₖ conjugates (1% modification of carboxyl groups, 0.5 nmol SIINFEKL/ml) w/or w/o 0.5 μg/ml CpG for 24 h. BMDCs were then washed by PBS, followed by co-culture with 5×10⁴ CFSE-labeled OT-I T cells for 3 days. Cells were then harvested, blocked with anti-CD16/32, and stained with anti-CD8-APC on ice for 30 min. Cells were washed and resuspended in 2 μg/ml DAPI solution and analyzed for fluorescence dilutions of CFSE by flow cytometry.

### Lymph Node (LN) Draining In Vivo

C57BL/6 mice were subcutaneously injected at two sides of tail base with PGA-SS-CSSSIINFEKL-FITC (1% modification of carboxyl groups in 30 kD or 120 kD PGA, 20 nmol peptide dissolved in 100 μl PBS/mouse). Inguinal and axillary LNs were collected 6 h later, and fluorescence intensity of LNs was measured by IVIS (PerkinElmer).

### Elicitation of Antigen-Specific CD8⁺ T Cells In Vive

For immunization study with SIINFEKL, C57BL/6 mice (n=3) were subcutaneously immunized with PBS or PGA₁₂₀ₖ-SS-CSSSIINFEKL (1% modification of carboxyl groups, 40 nmol SIIINFEKL/mouse) plus CpG (50 μg/mouse) for four times at two-week interval. For immunization study with ASMTNMELM, C57BL/6 mice (n=5) were subcutaneously immunized with PBS, soluble peptide plus CpG, or PGA₁₂₀ₖ-MAL-CSSASMTNMELM (5% modification of carboxyl groups) plus CpG for four times at one-week interval. Doses of the peptide and CpG were 20 nmol/mouse and 50 μg/mouse, respectively. Percents of antigen-specific CD8⁺ T cells among periphery CD8⁺ T cells were quantified by a tetramer staining assay one week after each dose. Briefly, periphery blood was collected and suspended in ACK lysis buffer to remove red blood cells. The remaining mononuclear cells were blocked with anti-CD16/32, and stained with a PE-labeled SIINFEKL-H-2K^{b}-tetramer or ASMTNMELM-H-2D^{b}-tetramer on ice for 30 min, followed by staining with anti-CD8-APC on ice for another 20 min. Cells were washed and resuspended in 2 μg/ml DAPI solution and analyzed by flow cytometry.

### Results

### Controlled Synthesis of PGA-Peptide Conjugates

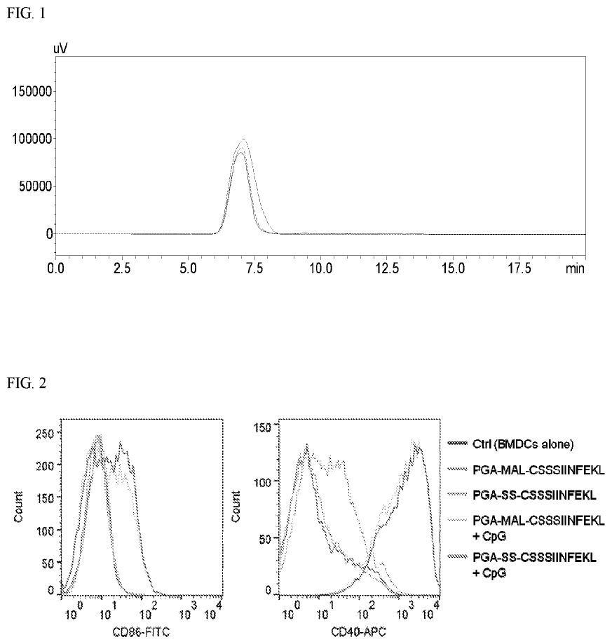

Linear PGA polymer was modified with antigen peptides by EDC/NHS chemistry. 1%-5% molar modification of total carboxyl groups was achieved by using 10-fold molar excess more EDC and NHS reagents than PDP or MAL linkers. However, modification ratios >5% decreased aqueous solubility of PGA-peptide conjugates, especially with water-insoluble peptides. Peptides modified with an N-terminal cysteine reacted with PDP or MAL, forming PGA-peptide conjugates linked by a reduction-sensitive (disulfide) and non-sensitive bond, respectively. These conjugates showed similar Mw distributions as the original PGA polymer, as shown by their similar retention profiles measured by GPC (FIG. 1).

### PGA-Peptide Conjugates Plus CpG Activate BMDCs In Vitro

Activation of BMDCs was investigated by measuring expression of co-stimulatory markers including CD86 and CD40 on DCs by flow cytometry. As shown in FIG. 2, PGA-MAL-CSSSIINFEKL alone failed to activate DCs, while PGA-SS-CSSSIINFEKL slightly increased CD40 expression in DCs. Addition of CpG resulted in significant DC activation.

### The Disulfide Linker Promotes Antigen Cross-Presentation In Vitro

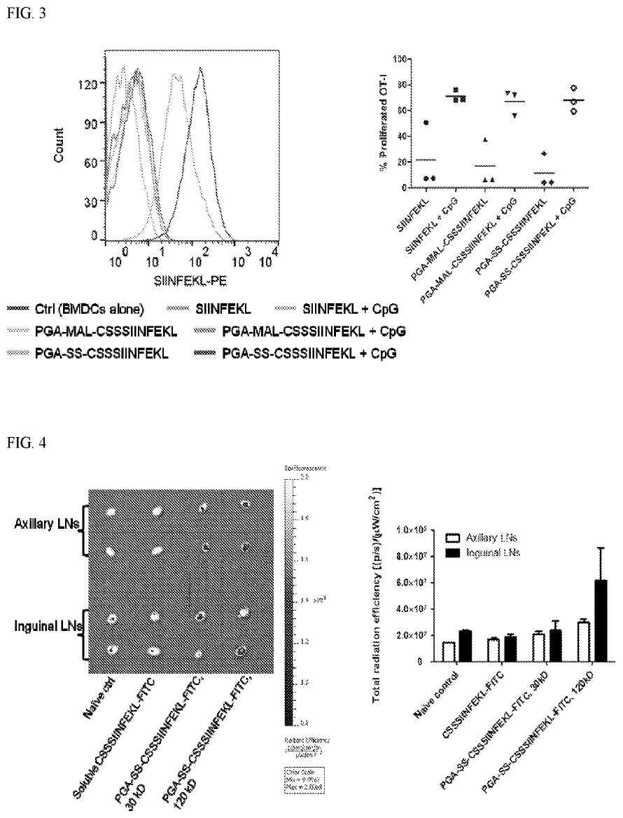

Cross-presentation of peptide antigens was investigated with the model antigen SIINFEKL. As shown in FIG. 3A, PGA-SS-CSSSIINFEKL induced significantly higher presentation of SIINFEKL on DCs than soluble peptide and PGA-MAL-CSSSIINFEKL, suggesting the reduction-sensitive disulfide linker promoted antigen cross-presentation, which was further increased by CpG. Cross-presentation of antigens was also measured by in vitro proliferation of OT-I T cells, which express the T-cell receptor specific for SIINFEKL. As shown in FIG. 3B, BMDCs pre-activated by either soluble peptide or PGA-peptide conjugates in the presence of CpG led to similarly high levels (>60%) of proliferation of OT-I T cells.

### High Mw PGA Promotes LN Draining In Vivo

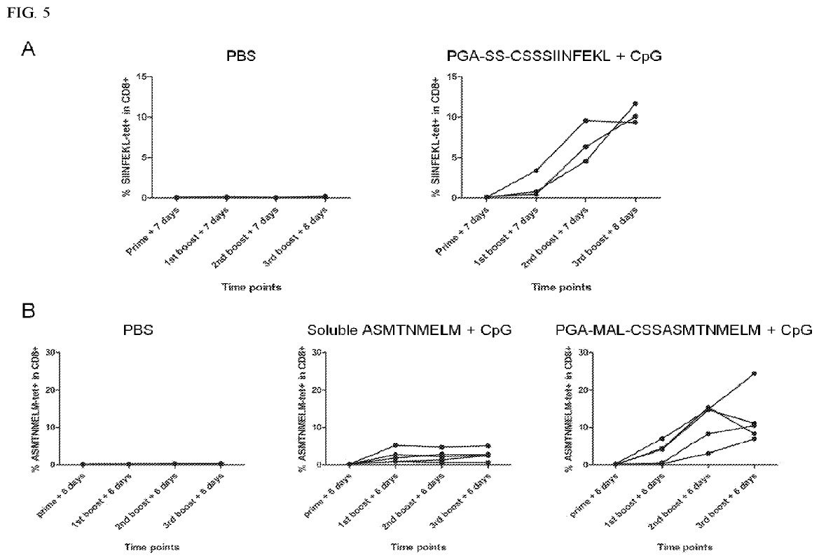

LN draining of antigen peptides following subcutaneous immunization in mice was measured using FITC-labeled SIINFEKL (FIG. 4). Compared to soluble peptide, PGA-peptide conjugates improved LN draining, and draining efficiency further increased as Mw of PGA increased from 30 kD to 120 kD.

### PGA-Peptide and PGA-Neo-Antigen Conjugates Plus CpG Elicit Antigen-Specific T-Cell Immune Responses In Vivo

PGA-peptide conjugates were admixed with CpG to form a peptide vaccine. Four doses of subcutaneous immunization of PGA₁₂₀ₖ-SS-CSSSIINFEKL plus CpG successfully elicited robust levels of SIINFEKL-specific CD8⁺ T cells in vivo (FIG. 5A). Next, the vaccine platform was tested using a tumor antigen peptide ASMTNMELM, which is a neo-antigen epitope derived from murine colon adenocarcinoma MC38 model. Compared with soluble peptide vaccine, PGA₁₂₀ₖ-MAL-CSSSIINFEKL plus CpG elicited higher levels of antigen-specific CD8⁺ T cells in vivo after four doses (FIG. 5B).

Vaccination with PGA-Neo-Antigen Conjugates Plus CpG Protect Animal Against Tumor Growth In Vivo

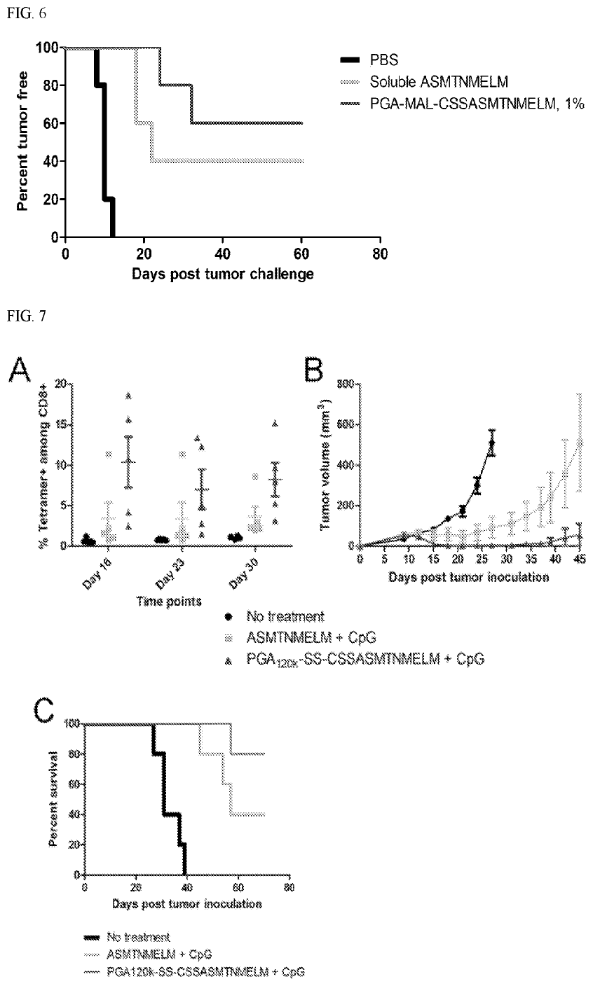

PGA-ASMTNMELM conjugate plus CpG was used to immunized mice 4 times, after which the animals were challenged with 10\hat{ }5 MC38 colon carcinoma cells. Mice immunized with PGA-ASMTNMELM exhibited significantly delayed tumor growth, compared with mice immunized with free antigen vaccine group (FIG. 6).

### Example II

This example demonstrates that intratumoral delivery of PGA-peptide conjugates plus CpG eliminates MC38 tumors.

### Methods

C57BL/6 mice (n=5) were subcutaneously inoculated with 5×10⁵ MC38 tumor cells on day 0, followed by a single intratumoral dose of soluble ASMTNMELM peptide or PGA₁₂₀ₖ-SS-CSSASMTNMELM (10 μg peptide/mouse), plus CpG (15 μg/mouse) on day 9, and the tetramer staining assay in PBMCs on days 16, 23, and 30. Tumor volumes were measured every three days and calculated as 0.5×[tumor length]×[tumor width]².

### Results

Therapeutic efficacy of the neo-antigen vaccine was tested on the MC38 tumor model. Compared to the admix of soluble peptide and CpG, a single intratumoral dose of PGA₁₂₀ₖ-SS-CSSASMTNMELM plus CpG elicited higher levels of antigen-specific CD8+ T cells, which were durable within 3 weeks after dosing (FIG. 7A), and regressed tumors in 80% of treated mice (FIG. 7B) with improved animal survival (FIG. 7C).

### Example III

This example describes cancer immunotherapy with PEI-neo-antigen peptide conjugates.

### Materials and Instruments

Polyethyleneimine (PEI, MW 25,000), dimethyl sulfoxide (DMSO), succinimidyl 3-(2-pyridyldithio)propionate (SPDP), 4′,6-Diamidine-2′-phenylindole dihydrochloride (DAPI) were purchased from Sigma-Aldrich. Methoxy poly(ethyleneglycol) propionic acid N-hydroxysuccinimide (MW 5,000, Methoxy-PEG-NHS) was purchased from Nanocs. Dithiothreitol (DTT) was obtained from Thermo Scientific. CpG (CpG 1826, 5′-tccatgacgttcctgacgtt-3′) was obtained from Integrated DNA Technology. Antigen peptide CSSASMTNMELM was supplied by RS synthesis. Antigen peptide ASMTNMELM and granulocyte-macrophage colony-stimulating factor (GM-CSF) were supplied by Genscript. RPMI 1640, penicillin-streptomycin (PS), beta-mercaptoethanol (b-ME), and ACK lysis buffer were obtained from Gibco. Fetal bovine serum (FBS) was obtained from Corning. Cell Counting Kit-8 (CCK-8) was purchased from Dojindo Laboratories. IL-12p70 ELISA kit was obtained from R&D system. PE-labeled tetramer (H-2D^{b}-restricted ASMTNMELM) was provided by NIH Tetramer Core Facility. Anti-CD8-APC antibody was purchased from BD Biosciences. Gel permeation chromatography was performed using High Performance Liquid Chromatograph (Shimadzu) equipped with TSKgel G3000SWxl column (Tosoh Bioscience LLC). UV-Vis absorbance was measured using BioTek synergy neo microplate reader. Flow cytometry was performed using ZE5 Cell Analyzer (Bio-Rad), and the data were analyzed using FlowJo 10.4 software.

### Synthesis of PEI-SS-CSSASMTNMELM

PEI (2.5 mg, 0.1 μmol) and methoxy-PEG-NHS (7.5 mg, 1.5 μmol) were mixed overnight in 400 μl DMSO. SPDP (2 mg, 6.4 μmol) in 200 μl DMSO was added and stirred for 2 h, and the neo-antigen peptide CSSASMTNMELM (7.5 mg, 5.8 μmol) in 750 μl DMSO was added and further reacted overnight at room temperature with vigorous stirring. The mixture was then dialyzed 5 times against DI water using Amicon ultra 10 KDa MW cutoff centrifugal filters. The purified PEI-SS-CSSASMTNMELM was freeze-dried and re-constituted in DI water at 5 mg/ml.

### In Vitro Cell Experiments Using Bone Marrow-Derived Dendritic Cells (BMDCs)

BMDCs were plated at a density of 1×10⁵ cells/well in 96 well plates in RPMI 1640 supplemented with 10% FBS and 1% PS, and incubated overnight at 37° C. under 5% CO₂. For cytotoxicity assessment, cells were incubated with either free form (CSSASMNTMELM) or PEI conjugate (PEI-SS-CSSASMTNMELM) of the neo-antigen peptide for 24 h at concentrations of 1, 5, 10, 20, 50, 100 μg/ml. The viability was measured by adding CCK-8 to each well of the plate according to manufacturer's instruction followed by absorbance measurement at 450 nm using a microplate reader after 2 h incubation. Relative viability was calculated as to the ratio of the absorbance to the non-sample treated cells (0 μg/ml). To investigate BMDC activation, cells were treated with PBS or PEI-SS-CSSASMTNMELM, PBS+CpG, PEI-SS-CSSASMTNMELM+CpG (2 μg/ml PEI-SS-CSSASMTNMELM and 1 μg/ml CpG) for 2 h. The fresh cell culture media were placed after sample washing and collected for cytokine analysis after further 24 h incubation. The concentration of IL-12p70 in the media was measured using ELISA kit by following the manufacturer's instruction.

### Animal Study

Animal studies were conducted using female C57BL/6 mice (5-6 weeks) purchased from The Jackson Laboratory (USA). Animals were cared for following federal, state, and local guidelines. All work performed on animals was in accordance with and approved by University Committee on Use and Care of Animals at University of Michigan, Ann Arbor. The animal study protocol was reviewed and approved by the Institutional Animal Care and Use Committee at the University of Michigan.

### In Vivo Immunization for Elicitation of Neo-Antigen-Specific CD8+ Cells

C57BL/6 mice were immunized subcutaneously at the tail base with 100 μl PBS solution of ASMTNMELM+CpG or PEI-SS-CSSASMTNMELM+CpG (10 μg ASMTNMELM and 15 μg CpG). Mice were immunized three times with a weekly interval while the control mice did not receive any samples (“no treatment”). Bloods were collected on day 7 after the last immunization by submandibular bleeding, and peripheral blood mononuclear cells (PBMCs) was obtained after removal of red blood cells using ACK lysis buffer. PBMCs were stained with PE-labeled tetramer and anti-CD8-APC antibody for the percentages of neo-antigen-specific CD8+ T cells. Flow cytometric analysis was performed after suspending cells in DAPI solution and gating out DAPI-positive populations to exclude dead cells.

### In Vivo Cancer Immunotherapy Against Established MC38 Tumors

C57BL/6 mice were subcutaneously injected with 5×10⁵ MC38 colon carcinoma cells (a gift from W. Zou, University of Michigan) into the right flank and randomly sorted for treatment after 9 days when the tumor volumes reached approximately 50-70 mm³. Fifty μl PBS solution of ASMTNMELM, CpG, ASMTNMELM+CpG, PEI-SS-CSSASMTNMELM+CpG (10 μg ASMTNMELM and 15 μg CpG), or blank PBS (50 μl) were directly injected into tumor every week for total three times. PBMCs were collected on day 7 after the first sample injection and tetramer staining was performed for flow cytometric analysis of neo-antigen-specific CD8+ T cells. The sizes of the tumors were measured every three days using a digital caliper, and the tumor volume was estimated by ellipsoidal calculation as V=(width)²×length×π/6. The mice were euthanized when the tumors reached the maximum permitted size (1.5 cm in any dimension) or ulcerations occurred.

### Synthesis of PEI-Neo-Antigen Conjugates

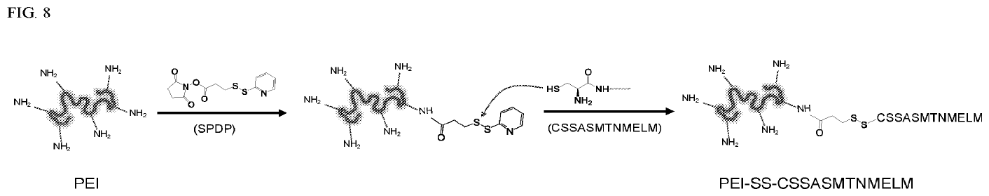

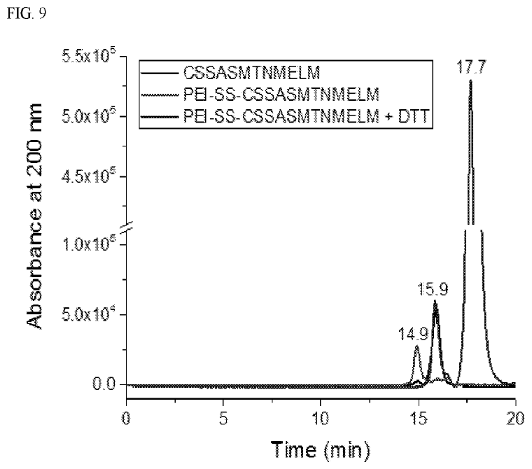

PEI was covalently conjugated with the neo-antigen peptide CSSASMTNMELM using SPDP cross-linker that introduces reduction-sensitive disulfide bond between PEI and the peptide (PEI-SS-CSSASMTNMELM) (FIG. 8). GPC analysis showed that the retention time of the peptide changed from 15.9 min for free peptide to 14.9 min for the PEI conjugate due to the gain of PEI molecular weight (FIG. 9). The PEI conjugate exhibited original retention profile of the peptide at 15.9 min after DTT treatment, while DTT was separately eluted at 17.7 min. The increase in the apparent molecular weight of the peptide was reversed via cleavage of disulfide bond by DTT treatment (FIG. 9), confirming the successful synthesis of disulfide-linked PEI-neo-antigen conjugate.

Biocompatibility of PE-Neo-Antigen Conjugates and their Immune Activation

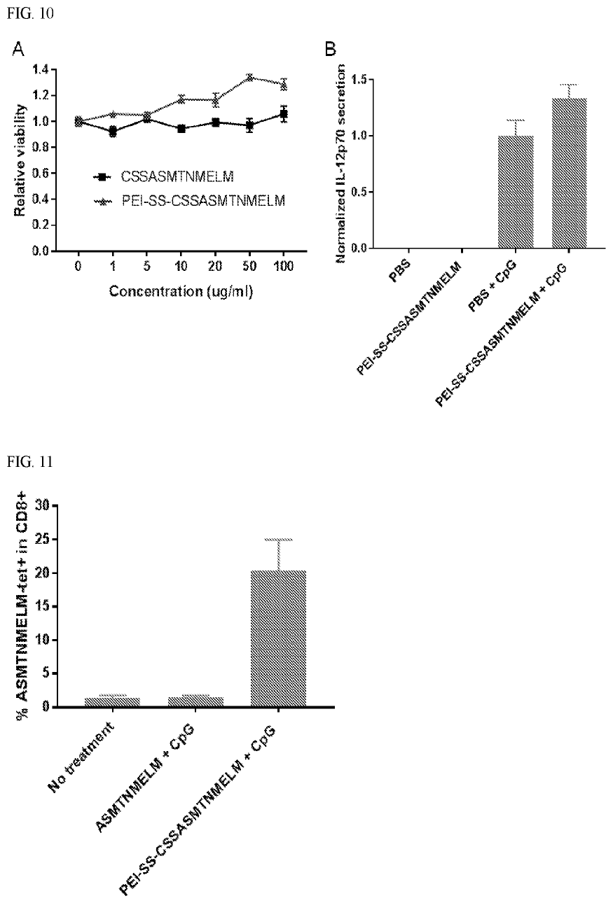

PEI-peptide conjugates were biocompatible, as BMDCs maintained their viability after 24 h incubation with either the free peptide or PEI conjugates at the concentration as high as 100 μg/ml (FIG. 10A). PBS or PEI conjugate alone failed to induce activation of BMDCs, while the addition of CpG resulted in significant activation as measured by secretion of pro-inflammatory cytokine IL-12p70 (FIG. 10B).

### PEI-Neo-Antigen Conjugates Plus CpG Elicits Antigen-Specific T Cell Responses in Vivo

The neo-antigen vaccine was formed by admixing CpG with either soluble neo-antigen or PEI-neo-antigen conjugates. As shown in FIG. 11, PEI-neo-antigen conjugates plus CpG elicited strong neo-antigen-specific CD8+ T cell response in vivo after three doses of subcutaneous immunization in mice, whereas soluble neo-antigen plus CpG induced only the basal level of the response that is similar to no treatment control.

### PEI-Neo-Antigen Conjugates Plus CpG Vaccination Regresses Established Tumors and Allows Long-Term Tumor-Free Survival In Vivo

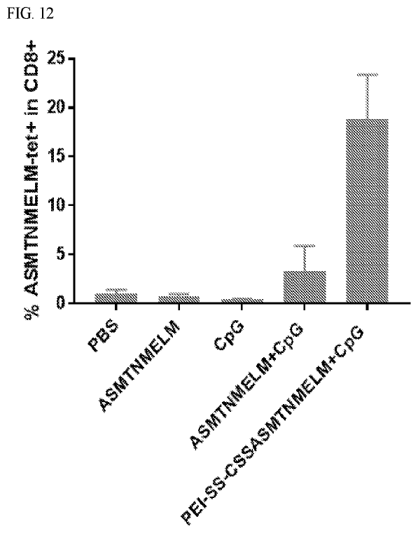

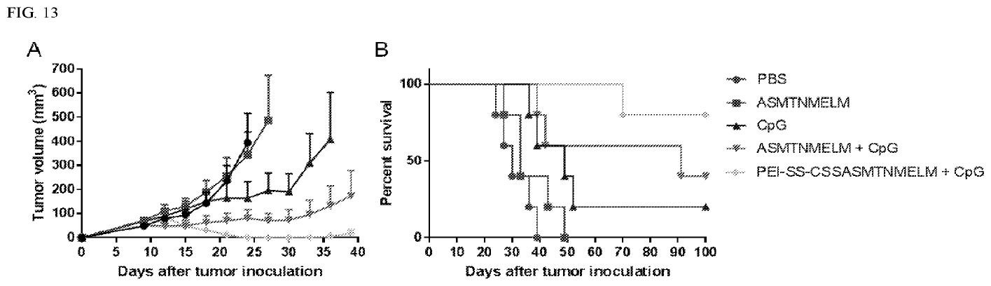

The anti-tumor efficacy of neo-antigen vaccine was investigated in vivo by direct administrations into established MC38 tumors. The single component, either neo-antigen or CpG alone, failed to elicit neo-antigen-specific CD8+ T cell response above the basal level of PBS. Importantly, vaccination with PEI-neo-antigen conjugates plus CpG elicited robust neo-antigen-specific CD8+ T cell responses, with 5.7-fold greater frequency of ASMTNMELM-tetramer+CD8+ T cells compared with that induced by soluble neo-antigen plus CpG (FIG. 12). After three doses of vaccination with PEI-neo-antigen conjugates plus CpG, 80% of MC38 tumor-bearing mice exhibited regression of established tumors, resulting in tumor-free survival for at least 100 days (FIG. 13A,B). In contract, intratumoral injection of CpG or soluble neo-antigen plus CpG slowed tumor progression, but only resulted in ˜20% and ˜40% survival rates. All animals treated with PBS or neo-antigen alone all succumbed to tumors within 50 days.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition comprising a polymer associated with a biomacromolecule agent,
wherein associated is selected from one or more of complexed, conjugated, encapsulated, adsorbed, and admixed.

2-7. (canceled)

8. The composition of claim 1,
wherein the polymer is selected from the group consisting of poly(L-glutamic acid (PGA), polyethyleneimine (PEI), poly(gamma-glutamic acid), polyglycolic acid, polyglycolic acid trimethyl-carbonates, polyglycolic acidtrimethyl-carbonates, polyglycolides, copolymers of the polylactides and polyglycolides, polylactic acid (PLLA), poly(lactic-co-glycolic acid) poly(D,L lactic-co-glycolide) (PLGA), PLGA derivatives, polyacrylic acid, polyethylene glycol (PEG), PEG derivatives, methoxy polyethylene glycol (mPEG), poly(D,L-lactide)-block-methoxypolyethylene glycol (diblock), polyethyleneoxide-propyleneoxide, polyethylene oxide (PEO), polyether esters (e.g., polyethyleneoxide, polyalkeneoxalates, polyorthoesters as well as their copolymers), poly-L-lysine, chitosan, hyaluronic acid, fatty acids, fatty acyls, polyamine, polyvinyl alcohol, polyvinylalcoholes, polyvinyl pyrrolidinone, acrylic acid ethylene co-polymer, poly(polypropylacrylic acid) (PPAA), polyacrylic acid, methoxy polyethylene, albumin, human serum albumin, cellulose derivatives, cyclodextrins and their derivatives (e.g., including polytryptophan, polytyrosine, polypropyleneoxide), folate linked cyclodextrin, folate linked cyclodextran, β-cyclodextrines, alginate, carrageenan, pectin, dextran, collagen, collagen-N-hydroxysuccinimide, polyaniline, polyalanine, polytryptophan, polytyrosine, polyamino acids, amino sugars, glycerolipid, glycerophospholipid, sphinglipid, sterol lipid, prenol lipid, saccarolipid, polyketide, glucosamines, lipopolysaccarides, aminopolysaccarides, poly(allylamines), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(ethyleneimine), poly(beta-aminoester), poly(hydroxyethyl methacrylate), polyacrylamide, polyethyloxazole, polysaccharides (e.g., dextran), linear and branched polyethylenimine, polyamidoamine, polypropylenimine, polyallylamine, spermine, polyvalerolactones, poly-ε-decalactones, polylactides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides (e.g., polymaleic anhydrides), polyhydroxymethacrylates, fibrin, polycyanoacrylates, polycaprolactonedimethylacrylates, poly-β-maleic acid, polycaprolactonebutyl-acrylates, multiblock polymers (e.g., oligocaprolactonedioles and oligodioxanonedioles), polyetherester multiblock polymers (e.g., PEG and poly(butyleneterephtalates)), polypivotolactones, polycaprolactone-glycolides, poly(g-ethylglutamate), poly(DTH-iminocarbonate), poly(DTE-co-DT-carbonate), poly(bisphenol-A-iminocarbonate), polyorthoesters, polytrimethylcarbonates, polyiminocarbonates, poly(N-vinyl)-pyrrolidone, polyesteramides, glycolated polyesters, polyphosphoesters, polyphosphazenes, poly[p-carboxyphenoxy]propane], polyhydroxypentanoic acid, soft polyurethanes, polyurethanes having amino acid residues in the backbone, fibrinogen, starch, protein based polymers, polyamino acids, synthetic polyamino acids, zein, modified zein, polyhydroxyalkanoates, pectic acid, actinic acid, modified and non modified fibrin and casein, carboxymethylsulfate, heparansulfates, heparin, chondroitinesulfate, copolymers with PEG and polypropyleneglycol, gummi arabicum, guar, gelatine, lipids and lipoids, polymerizable oils having a low degree of cross-linking, modifications, grafted polymers, and copolymers and/or mixtures of the aforementioned substances.

9. The composition of claim 1, wherein the biomacromolecule agent is selected from a peptide and/or a nucleic acid molecule.

10. The composition of claim 1, wherein the biomacromolecule agent is a peptide selected from the group consisting of 177Lu-DOTA0-Tyr3-Octreotate, Abarelix acetate, ADH-1, Afamelanotidec, melanotan-1, CUV1647, Albiglutide, Aprotinin, Argipressin, Atosiban acetate, Bacitracin, Bentiromide, a BH3 domain, Bivalirudin, Bivalirudin trifluoroacetate hydrate, Blisibimod, Bortezomib, Buserelin, Buserelin acetate, Calcitonin, Carbetocin, Carbetocin acetate, Cecropin A and B, Ceruletide, Ceruletide diethylamine, Cetrorelix, Cetrorelix acetate, Ciclosporine, Cilengitidec, EMD121974, Corticorelin acetate injection, hCRF, Corticorelin ovine triflutate, corticorelin trifluoroacetate, Corticotropin, Cosyntropin, ACTH 1-24, tetracosactide hexaacetate, Dalbavancin, Daptomycin, Degarelix acetate, Depreotide trifluoroacetate (plus sodium pertechnetate), Desmopressin acetate, Desmopressin DDAVP, Dulaglutide, Ecallantide, Edotreotide (plus yttrium-90), Elcatonin acetate, Enalapril maleate (or 2-butanedioate), Enfuvirtide, Eptifibatide, Exenatide, Ganirelix acetate, Glatiramer acetate, Glutathion, Gonadorelin, Gonadorelin acetate, GnRH, LHRH, Goserelin, Goserelin acetate, Gramicidin, Histrelin acetate, Human calcitonin, Icatibant, Icatibant acetate, IM862, oglufanide disodium, KLAKLAK, Lanreotide acetate, Lepirudin, Leuprolide, Leuprolide acetate, Leuprorelin, Liraglutide, Lisinopril, Lixisenatide, Lypressin, Magainin2, MALP-2Sc, macrophage-activating lipopeptide-2 synthetic, Nafarelin acetate, Nesiritide, NGR-hTNF, Octreotide acetate, Oritavancin, Oxytocin, Pasireotide, Peginesatide, Pentagastrin, Pentetreotide (plus indium-111), Phenypressin, Pleurocidin, Pramlintide, Protirelin, thyroliberin, TRH, TRF, Salmon calcitonin, Saralasin acetate, Secretin (human), Secretin (porcine), Semaglutide, Seractide acetate, ACTH, corticotropin, Sermorelin acetate, GRF 1-29, Sinapultide, KL4 in lucinactant, Sincalide, Somatorelin acetate, GHRH, GHRF, GRF, Somatostatin acetate, Spaglumat magnesium (or sodium) salt, Substance P, Taltirelin hydrate, Teduglutide, Teicoplanin Telavancin, Teriparatide, Terlipressin acetate, Tetracosactide, Thymalfasin, thymosin a-1, Thymopentin, Trebananib, Triptorelin, Triptorelin pamoate, Tyroserleutide, Ularitide, Vancomycin, Vapreotide acetate, Vasoactive intestinal peptide acetate, Vx-001c, TERT572Y, Ziconotide acetate, α5-α6 Bax peptide, β-defensin, an Adrenocorticotropic Hormone (ACTH), a growth hormone peptide, a Melanocyte Stimulating Hormone (MSH), Oxytocin, Vasopressin, Corticotropin Releasing Factor (CRF), a CRF-related peptide, a Gonadotropin Releasing Hormone Associated Peptide (GAP), Growth Hormone Releasing Factor (GRF), Lutenizing Hormone Release Hormone (LH-RH), an orexin, a Prolactin Releasing Peptide (PRP), a somatostatin, Thyrotropin Releasing Hormone (THR), a THR analog, Calcitonin (CT), a CT-precursor peptide, a Calcitonin Gene Related Peptide (CGRP), a Parathyroid Hormone (PTH), a Parathyroid Hormone Related Protein (PTHrP), Amylin, Glucagon, Insulin, an Insulin-like peptide, NeuroPeptide Y (NPY), a Pancreatic Polypeptide (PP), Peptide YY (PYY), Cholecystokinin (CCK), a CCK-related peptide, Gastrin Releasing Peptide (GRP), Gastrin, a Gastrin-related peptide, a Gastrin inhibitory peptide, Motilin, Secretin, Vasoactive Intestinal Peptide (VIP), a VIP-related peptide, an Atrial-Natriuretic Peptide (ANP), a Brain Natriuretic Peptide (BNP), a C-Type Natriuretic Peptide (CNP), a tachykinin, an angiotensin, a renin substrate, a renin inhibitor, an endothelin, an endothelin-related peptide, an opioid peptide, a thymic peptide, an adrenomedullin peptide, an allostatin peptide, an amyloid beta-protein fragment, an antimicrobial peptide, an antioxidant peptide, an apoptosis related peptide, a Bag Cell Peptide (BCPs), Bombesin, a bone Gla protein peptide, a Cocaine and Amphetamine Related Transcript (CART) peptide, a cell adhesion peptide, a chemotactic peptide, a complement inhibitor, a cortistatin peptide, a fibronectin fragment, a fibrin related peptide, FMRF, a FMRF amide-related peptide (FaRP), Galanin, a Galanin-related peptide, a growth factor, a growth factor-related peptide, a G-Therapeutic Peptide-Binding Protein fragment, Gualylin, Uroguanylin, an Inhibin peptide, Interleukin (IL), an Interleukin Receptor protein, a laminin fragment, a leptin fragment peptide, a leucokinin, Pituitary Adenylate Cyclase Activating Polypeptide (PAPCAP), Pancreastatin, a polypeptide repetitive chain, a signal transducing reagent, a thrombin inhibitor, a toxin, a trypsin inhibitor, a virus-related peptide, an adjuvant peptide analog, Alpha Mating Factor, Antiarrhythmic Peptide, Anorexigenic Peptide, Alpha-1 Antitrypsin, Bovine Pineal Antireproductive Peptide, Bursin, C3 Peptide P16, Cadherin Peptide, Chromogranin A Fragment, Contraceptive Tetrapeptide, Conantokin G, Conantokin T, Crustacean Cardioactive Peptide, C-Telopeptide, Cytochrome b588 Peptide, Decorsin, Delicious Peptide, Delta-Sleep-Inducing Peptide, Diazempam-Binding Inhibitor Fragment, Nitric Oxide Synthase Blocking Peptide, OVA Peptide, Platelet Calpain Inhibitor (P1), Plasminogen Activator Inhibitor 1, Rigin, Schizophrenia Related Peptide, Sodium Potassium Atherapeutic Peptidase Inhibitor-1, Speract, Sperm Activating Peptide, Systemin, a Thrombin receptor agonist, Tuftsin, Adipokinetic Hormone, Uremic Pentapeptide, Antifreeze Polypeptide, Tumor Necrosis Factor (TNF), Leech [Des Asp10]Decorsin, L-Ornithyltaurine Hydrochloride, P-Aminophenylacetyl Tuftsin, Ac-Glu-Glu-Val-Val-Ala-Cys-pNA, Ac-Ser-Asp-Lys-Pro, Ac-rfwink-NH2, Cys-Gly-Tyr-Gly-Pro-Lys-Lys-Lys-Arg-Lys-Val-Gly-Gly, D-Ala-Leu, D-D-D-D-D, D-D-D-D-D-D, N-P-N-A-N-P-N-A, V-A-I-T-V-L-V-K, V-G-V-R-V-R, V-I—H-S, V-P-D-P-R, Val-Thr-Cys-Gly, R-S-R, Sea Urchin Sperm Activating Peptide, a SHU-9119 antagonist, a MC3-R antagonist, a MC4-R antagonist, Glaspimod, HP-228, Alpha 2-Plasmin Inhibitor, APC Tumor Suppressor, Early Pregnancy Factor, Gamma Interferon, Glandular Kallikrei N-1, Placental Ribonuclease Inhibitor, Sarcolecin Binding Protein, Surfactant Protein D, Wilms' Tumor Suppressor, GABAB 1b Receptor Peptide, Prion Related Peptide (iPRP13), Choline Binding Protein Fragment, Telomerase Inhibitor, Cardiostatin Peptide, Endostatin Derived Peptide, Prion Inhibiting Peptide, N-Methyl D-Aspartate Receptor Antagonist, C-Peptide-Analog, myelin basic protein (MBP), myelin oligodendrocyte protein (MOG), and proteolipid protein (PLP), insulin, and their mimotopes;
wherein the nucleic acid molecule selected from RNA, siRNA, microRNA, interference RNA, mRNA, replicon mRNA, RNA-analogues, DNA, and DNA analogues.

13. (canceled)

14. The composition of claim 1, wherein the biomacromolecule agent is a peptide, wherein the peptide is an antigen, wherein the antigen is selected from the group consisting of a peptide based antigen, a protein based antigen, an inactivated organism based antigen, an attenuated organism based antigen, a viral antigen, a bacterial antigen, a parasite antigen, an antigen derived from an allergen, a tumor antigen, a neo-antigen, a polysaccharide based antigen, a saccharide based antigen, a lipid based antigen, a glycolipid based antigen, and a nucleic acid based antigen.

15. The composition of claim 1, wherein the biomacromolecule agent is a peptide, wherein the peptide is an antigen,
wherein the antigen is a tumor antigen selected from the group consisting of alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGS), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), human EGFR protein or its fragments, such as human EGFR residues 306-325 (SCVRACGADSYEMEEDGVRK) and residues 897-915 (VWSYGVTVWELMTFGSKPY), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, TPS, WT1 (and WT1-derivaed peptide sequences: WT1 126-134 (RMFP NAPYL), WT1 122-140 (SGQARMFPNAPYLPSCLES), and WT1 122-144 (SGQARMFPNAPYLPSCLESQPTI)), MUC1 (and MUC1-derived peptides and glycopeptides such as RPAPGS, PPAHGVT, and PDTRP), LMP2, EGFRvIII, Idiotype, GD2, Ras mutant, p53 mutant, Proteinase3 (PR1), Survivin, hTERT, Sarcoma translocation breakpoints, EphA2, EphA4, LMW-PTP, PAP, ML-IAP, AFP, ERG (TMPRSS2 ETS fusion gene), NA17, PAX3, ALK, Androgen receptor, Cyclin B1, Polysialic acid, MYCN, RhoC, TRP-2, GD3, Fucosyl GMI, Mesothelin, sLe (animal), CYP1B1, PLAC1, GM3, BORIS, Tn, GloboH, NY-BR-1, RGS5, SART3, STn, Carbonic anhydrase IX, PAX5, OY-TES1, Sperm protein 17, LCK, HMWMAA, AKAP-4, XAGE 1, B7H3, Legumain, Tie 2, Page4, VEGFR2, MAD-CT-1, FAP, PDGFR-alpha, PDGFR-β, MAD-CT-2, Fos-related antigen 1, ERBB2, Folate receptor 1 (FOLR1 or FBP), IDH1, IDO, LY6K, fms-related tyro-sine kinase 1 (FLT1, best known as VEGFR1), KDR, PADRE, TA-CIN (recombinant HPV16 L2E7E6), SOX2, and aldehyde dehydrogenase,
wherein the antigen is against PCSK9, M30, M27, Adpgk, CSSASMTNMELM, and/or ASMTNMELM.

16. The composition of claim 1, wherein the biomacromolecule agent is a peptide, wherein the peptide is an antigen, wherein the antigen is derived from a self-antigen, wherein the composition is used to induce immune tolerance against autoimmune diseases.

17. The composition of claim 1, wherein the polymer is further associated with an adjuvant,
wherein associated is selected from one or more of complexed, conjugated, encapsulated, adsorbed, and admixed.

18-22. (canceled)

23. The composition of claim 17, wherein the adjuvant is selected from the group consisting of CPG, polyIC, poly-ICLC, 1018 ISS, aluminum salts (for example, aluminum hydroxide, aluminum phosphate), Amplivax, BCG, CP-870,893, CpG7909, CyaA, dSLIM, Cytokines (such as GM-CSF, IL-2, IFN-a, Flt-3L), IC30, IC31, Imiquimod, ImuFact IMP321, IS Patch, ISS, ISCOMATRIX, Juylmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), 3M MEDI9197, glucopyranosyl lipid adjuvant (GLA), GLA-SE, CD1d ligands (such as C20:2, OCH, AH04-2, α-galatosylceramide, α-C-galatosylceramide,α-mannosylceramide, α-fructosylceramide, β-galatosylceramide, β-mannosylceramide), STING agonists (e.g. cyclic dinucleotides, including Cyclic [G(3′,5′)pA(3′,5′)p], Cyclic [G(2′,5′)pA(3′,5′)p], Cyclic [G(2′,5′)pA(2′,5′)p], Cyclic diadenylate monophosphate, Cyclic diguanylate monophosphate), CL401, CL413, CL429, Flagellin, RC529, E6020, imidazoquinoline-based small molecule TLR-7/8a (including its lipidated analogues), virosomes, AS01, AS02, AS03, AS04, AS15, IC31, CAF01, ISCOM, Cytokines (such as GM-CSF, IL-2, IFN-a, Flt-3L), bacterial toxins (such as CT, and LT), any derivative of an adjuvant, and any combination of adjuvant.

24-25. (canceled)

26. The composition of claim 1,
wherein the biomacromolecule agent is conjugated within the polymer a reduction insensitive linkage or a reduction sensitive linkage agent,
wherein the reduction insensitive linkage agent is a maleimide-thiol linkage,
wherein the reduction sensitive linkage agent is a pyridyldithiol propionate-thiol linkage.

27-31. (canceled)

32. The composition of claim 1, wherein the composition is used as a vaccine against a pathogen and/or wherein the composition is used within methods for cancer immunotherapy,
wherein the pathogen is selected from erythropoietic porphyries, T2 diabetes, antifibrinolytic, central diabetes insipidus, delaying the birth in case of threat of premature birth, antibiotic, cystic fibrosis, angina, anticoagulant in patients with unstable angina undergoing PTCA or PCI, systemic lupus erythematosus, hypercalcemia, osteoporosis, pagets disease, carbetocin works as an oxytocic, antihemorrhagic and uterotonic drug in the peripheral nervous system, prevention of uterine atony, induction, and control postpartum bleeding or haemorrhage, stimulant of the gastric secretion, for treat hormone-sensitive cancers of the prostate and breast, inhibition of premature LH surges in women undergoing controlled ovarian stimulation, immunosuppression in organ transplantation to prevent rejection, peritumoral brain edema, diagnosis of ACTHdependent Cushing's syndrome, allergies, ankylosing spondylitis, psoriasis, chorioditis, erythema, keratitis, sclerosis, dermatomyositis, rheumatoid arthritis, Stevens-Johnson Syndrome, ulcerative colitis, diagnosis of adrenocortical insufficiency, antibiotic, systemic infections caused by gram positive organisms, nocturnal enuresis, nocturia, and stoppage of bleeding or haemorrhage in haemophilia A patients, acute hereditary angioderma, postmenopausal osteoporosis, anti-parathyroid, Paget's disease, hypercalcaemia, hypertension, AIDS/HIV-1 infection, acute coronary syndrome, unstable angina undergoing PCI, Alzheimer's and Parkinson's disease, inhibition of premature LH surges in women undergoing controlled ovarian hyperstimulation, Relapsing-Remitting Multiple Sclerosis, hepatic insufficiency, wound healing, inflammation of respiratory tract, asthenia, release of follicle-stimulating hormone (FSH) and luteinizing hormone (LH) from the anterior pituitary, stimulate the secretion of gonadotropin during disturbances fertility, and diagnosis of the functional capacity and response of the gonadotropes of the anterior pituitary, for skin lesions, surface wounds and eye infections, postmenopausal osteoporosis, Paget's disease, hypercalcaemia, hereditary angioedema, immune system related diseases, acromegaly, anticoagulant, fibroids and endometriosis, central diabetes insipidus, Cushing's syndrome, diabetic foot ulcers, treatment of central precocious puberty, uterine fibriods and endometriosis, vasodilatory, natriuretic, diuretic and neurohormonal effects, acromegaly, carcinoid syndrome, acute bacterial skin and skin structure infections, initiation or improvement of uterine contractions, and control postpartum bleeding or haemorrhage, hematide Chronic kidney disease associated anemia, stomatitis, pharyngitis, diagnostic assessment of thyroid function, postmenopausal osteoporosis, hypercalcaemia diagnosis of pancreatic exocrine dysfunction, and gastrinoma, Zollinger-Ellison syndrome, prevention of RDS in premature infants, and meconium aspiration syndrome, acute variceal bleeding, allergic rhinitis and conjunctivitis, spinocerebellar degeneration/ataxia, Short Bowel Syndrome, antibiotic, bactericidal, teriparatide is the only anabolic (i.e., bone growing) agent indicated for use in postmenopausal women with osteoporosis, Cortrosyn Analogue of adrenocorticotrophic hormone (ACTH) used for diagnostic purposes, treatment of adrenal insufficiency, epilepsia, Chronic hepatitis B, chronic hepatitis C, primary and secondary immune deficiencies, acute decompensated heart failure, colitis, esophageal variceal bleeding in patients with cirrhotic liver disease and AIDS-related diarrhea, sarcoidosis and acute lung injury, and severe chronic pain.

33-34. (canceled)

35. A method of delivering a biomacromolecule agent to a subject, comprising administering to the subject a composition as described in claim 1.

36. A method of treating a condition, disorder and/or disease in a subject, comprising administering a composition as described in claim 1 to the subject.

37. The method of claim 36, wherein the disorder and/or disease is peripheral ischemia, cancer, an inflammatory disorder, autoimmune diseases, immune-related diseases, and/or a genetic disorder.

38. The method of claim 36, wherein the biomacromolecule is a peptide, wherein the peptide and condition, disorder and/or disease is selected from the group consisting of each row in Table 1.

39. The method of claim 36, wherein the condition, disorder and/or disease is selected from erythropoietic porphyries, T2 diabetes, antifibrinolytic, central diabetes insipidus, delaying the birth in case of threat of premature birth, antibiotic, cystic fibrosis, angina, anticoagulant in patients with unstable angina undergoing PTCA or PCI, systemic lupus erythematosus, hypercalcemia, osteoporosis, pagets disease, carbetocin works as an oxytocic, antihemorrhagic and uterotonic drug in the peripheral nervous system, prevention of uterine atony, induction, and control postpartum bleeding or haemorrhage, stimulant of the gastric secretion, for treat hormone-sensitive cancers of the prostate and breast, inhibition of premature LH surges in women undergoing controlled ovarian stimulation, immunosuppression in organ transplantation to prevent rejection, peritumoral brain edema, diagnosis of ACTHdependent Cushing's syndrome, allergies, ankylosing spondylitis, psoriasis, chorioditis, erythema, keratitis, sclerosis, dermatomyositis, rheumatoid arthritis, Stevens-Johnson Syndrome, ulcerative colitis, diagnosis of adrenocortical insufficiency, antibiotic, systemic infections caused by gram positive organisms, nocturnal enuresis, nocturia, and stoppage of bleeding or haemorrhage in haemophilia A patients, acute hereditary angioderma, postmenopausal osteoporosis, anti-parathyroid, Paget's disease, hypercalcaemia, hypertension, AIDS/HIV-1 infection, acute coronary syndrome, unstable angina undergoing PCI, Alzheimer's and Parkinson's disease, inhibition of premature LH surges in women undergoing controlled ovarian hyperstimulation, Relapsing-Remitting Multiple Sclerosis, hepatic insufficiency, wound healing, inflammation of respiratory tract, asthenia, release of follicle-stimulating hormone (FSH) and luteinizing hormone (LH) from the anterior pituitary, stimulate the secretion of gonadotropin during disturbances fertility, and diagnosis of the functional capacity and response of the gonadotropes of the anterior pituitary, for skin lesions, surface wounds and eye infections, postmenopausal osteoporosis, Paget's disease, hypercalcaemia, hereditary angioedema, immune system related diseases, acromegaly, anticoagulant, fibroids and endometriosis, central diabetes insipidus, Cushing's syndrome, diabetic foot ulcers, treatment of central precocious puberty, uterine fibriods and endometriosis, vasodilatory, natriuretic, diuretic and neurohormonal effects, acromegaly, carcinoid syndrome, acute bacterial skin and skin structure infections, initiation or improvement of uterine contractions, and control postpartum bleeding or haemorrhage, hematide Chronic kidney disease associated anemia, stomatitis, pharyngitis, diagnostic assessment of thyroid function, postmenopausal osteoporosis, hypercalcaemia, diagnosis of pancreatic exocrine dysfunction, and gastrinoma, Zollinger-Ellison syndrome, prevention of RDS in premature infants, and meconium aspiration syndrome, acute variceal bleeding, allergic rhinitis and conjunctivitis, spinocerebellar degeneration/ataxia, Short Bowel Syndrome, antibiotic, bactericidal, teriparatide is the only anabolic (i.e., bone growing) agent indicated for use in postmenopausal women with osteoporosis, Cortrosyn Analogue of adrenocorticotrophic hormone (ACTH) used for diagnostic purposes, treatment of adrenal insufficiency, epilepsia, Chronic hepatitis B, chronic hepatitis C, primary and secondary immune deficiencies, acute decompensated heart failure, colitis, esophageal variceal bleeding in patients with cirrhotic liver disease and AIDS-related diarrhea, sarcoidosis and acute lung injury, and severe chronic pain.

40. The method of claim 36, wherein the composition is coadministered with one or more of an adjuvant, a chemotherapy, an anti-immunosuppressive agent, and an immuno stimulatory agent,
wherein the immuno stimulatory agent is selected from the group consisting of anti-CTLA-4 antibody, anti-PD-1, anti-PD-L1, anti-TIM-3, anti-BTLA, anti-VISTA, anti-LAG3, anti-CD25, anti-CD27, anti-CD28, anti-CD137, anti-OX40, anti-GITR, anti-ICOS, anti-TIGIT, and inhibitors of IDO,
wherein the chemotherapeutic agent is one or more of the following: aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel, pilocarpine, prochloroperazine, rituximab, tamoxifen, taxol, topotecan hydrochloride, trastuzumab, vinblastine, vincristine and vinorelbine tartrate.

41-42. (canceled)

43. The composition of claim 1, wherein the composition comprising a polymer associated with a biomacromolecule agent is further associated with one or more of the following:
an additional composition comprising a polymer associated with an antigen;
an additional composition comprising a polymer associated with an adjuvant;
an additional composition comprising a polymer associated with a biomacromolecule agent;
a polymer;
a complexing agent;
a crosslinking agent; and
any agent capable of nanoparticle formation.


SEQUENCE LISTING (31 sequences)

SEQ ID NO: 1 - protein, 20 aa, Artificial Sequence
  Synthetic
SCVRACGADSYEMEEDGVRK

SEQ ID NO: 2 - protein, 19 aa, Artificial Sequence
  Synthetic
VWSYGVTVWELMTFGSKPY

SEQ ID NO: 3 - protein, 9 aa, Artificial Sequence
  Synthetic
RMFPNAPYL

SEQ ID NO: 4 - protein, 19 aa, Artificial Sequence
  Synthetic
SGQARMFPNAPYLPSCLES

SEQ ID NO: 5 - protein, 23 aa, Artificial Sequence
  Synthetic
SGQARMFPNAPYLPSCLESQPTI

SEQ ID NO: 6 - protein, 6 aa, Artificial Sequence
  Synthetic
RPAPGS

SEQ ID NO: 7 - protein, 7 aa, Artificial Sequence
  Synthetic
PPAHGVT

SEQ ID NO: 8 - protein, 5 aa, Artificial Sequence
  Synthetic
PDTRP

SEQ ID NO: 9 - protein, 14 aa, Artificial Sequence
  Synthetic
MALCSSSIINFEKL

SEQ ID NO: 10 - protein, 13 aa, Artificial Sequence
  Synthetic
SSCSSSIINFEKL

SEQ ID NO: 11 - protein, 8 aa, Artificial Sequence
  Synthetic
SIINFEKL

SEQ ID NO: 12 - protein, 15 aa, Artificial Sequence
  Synthetic
CSSSIINFEKLFITC

SEQ ID NO: 13 - protein, 17 aa, Artificial Sequence
  Synthetic
SSCSSSIINFEKLFITC

SEQ ID NO: 14 - protein, 9 aa, Artificial Sequence
  Synthetic
ASMTNMELM

SEQ ID NO: 15 - protein, 15 aa, Artificial Sequence
  Synthetic
MALCSSASMTNMELM

SEQ ID NO: 16 - protein, 14 aa, Artificial Sequence
  Synthetic
SSCSSASMTNMELM

SEQ ID NO: 17 - protein, 17 aa, Artificial Sequence
  Synthetic
PEISSCSSASMTNMELM

SEQ ID NO: 18 - protein, 6 aa, Artificial Sequence
  Synthetic
EEVVAC

SEQ ID NO: 19 - protein, 4 aa, Artificial Sequence
  Synthetic
SDKP

SEQ ID NO: 20 - protein, 13 aa, Artificial Sequence
  Synthetic
CGYGPKKKRKVGG

SEQ ID NO: 21 - protein, 5 aa, Artificial Sequence
  Synthetic
DDDDD

SEQ ID NO: 22 - protein, 6 aa, Artificial Sequence
  Synthetic
DDDDDD

SEQ ID NO: 23 - protein, 8 aa, Artificial Sequence
  Synthetic
NPNANPNA

SEQ ID NO: 24 - protein, 8 aa, Artificial Sequence
  Synthetic
VAITVLVK

SEQ ID NO: 25 - protein, 6 aa, Artificial Sequence
  Synthetic
VGVRVR

SEQ ID NO: 26 - protein, 4 aa, Artificial Sequence
  Synthetic
VIHS

SEQ ID NO: 27 - protein, 5 aa, Artificial Sequence
  Synthetic
VPDPR

SEQ ID NO: 28 - protein, 4 aa, Artificial Sequence
  Synthetic
VTCG

SEQ ID NO: 29 - protein, 11 aa, Artificial Sequence
  Synthetic
CSSSIINFEKL

SEQ ID NO: 30 - protein, 12 aa, Artificial Sequence
  Synthetic
CSSASMTNMELM

SEQ ID NO: 31 - protein, 9 aa, Artificial Sequence
  Synthetic
SIIINFEKL